SEQUENCE LISTING (118 sequences)

SEQ ID NO: 1 - skipped

SEQ ID NO: 2 - protein, 17 aa, synthetic construct
  VARIANT 1: note = D or A
  VARIANT 5: note = D, N or A
  VARIANT 6: note = Y or A
  VARIANT 7: note = D or A
  VARIANT 11: note = Y or A
  VARIANT 12: note = N or S
  VARIANT 13: note = E or Q
XINPXXXTTSXXXKFKG

SEQ ID NO: 3 - protein, 14 aa, synthetic construct
  VARIANT 3: note = Y or A
  VARIANT 8: note = Y or A
  VARIANT 9: note = Y or A
  VARIANT 13: note = D or A
  VARIANT 14: note = Y or A
GGXSGSSXXHPMXX

SEQ ID NO: 4 - protein, 17 aa, synthetic construct
  VARIANT 8: note = Y or A
  VARIANT 10: note = T or S
  VARIANT 12: note = Y or A
  VARIANT 15: note = Y or A
KSSQSLLXSXNXKNXLA

SEQ ID NO: 5 - protein, 7 aa, synthetic construct
  VARIANT 1: note = W or A
XASTRES

SEQ ID NO: 6 - protein, 9 aa, synthetic construct
  VARIANT 3: note = Y or A
  VARIANT 4: note = F or Y
  VARIANT 5: note = I or N
  VARIANT 6: note = Y or A
  VARIANT 8: note = R or Y
QQXXXXPXT

SEQ ID NO: 7 - protein, 17 aa, synthetic construct
  VARIANT 1: note = D or A
  VARIANT 5: note = D or A
  VARIANT 6: note = Y or A
  VARIANT 7: note = D or A
  VARIANT 11: note = Y or A
XINPXXXTTSXNEKFKG

SEQ ID NO: 8 - protein, 17 aa, synthetic construct
  VARIANT 8: note = Y or A
  VARIANT 15: note = Y or A
KSSQSLLXSTNQKNXLA

SEQ ID NO: 9 - protein, 9 aa, synthetic construct
  VARIANT 3: note = Y or A
  VARIANT 6: note = Y or A
QQXFIXPRT

SEQ ID NO: 10 - protein, 5 aa, synthetic construct
DYNMD

SEQ ID NO: 11 - protein, 17 aa, synthetic construct
DINPDYDTTSYNEKFKG

SEQ ID NO: 12 - protein, 14 aa, synthetic construct
GGYSGSSYYHPMDY

SEQ ID NO: 13 - protein, 17 aa, synthetic construct
KSSQSLLYSTNQKNYLA

SEQ ID NO: 14 - protein, 7 aa, synthetic construct
WASTRES

SEQ ID NO: 15 - protein, 9 aa, synthetic construct
QQYFIYPRT

SEQ ID NO: 16 - protein, 5 aa, synthetic construct
AYNMD

SEQ ID NO: 17 - protein, 5 aa, synthetic construct
DANMD

SEQ ID NO: 18 - protein, 5 aa, synthetic construct
DYNMA

SEQ ID NO: 19 - protein, 17 aa, synthetic construct
AINPDYDTTSYNEKFKG

SEQ ID NO: 20 - protein, 17 aa, synthetic construct
DINPAYDTTSYNEKFKG

SEQ ID NO: 21 - protein, 17 aa, synthetic construct
DINPDADTTSYNEKFKG

SEQ ID NO: 22 - protein, 17 aa, synthetic construct
DINPDYATTSYNEKFKG

SEQ ID NO: 23 - protein, 17 aa, synthetic construct
DINPDYDTTSANEKFKG

SEQ ID NO: 24 - protein, 14 aa, synthetic construct
GGASGSSYYHPMDY

SEQ ID NO: 25 - protein, 14 aa, synthetic construct
GGYSGSSAYHPMDY

SEQ ID NO: 26 - protein, 14 aa, synthetic construct
GGYSGSSYAHPMDY

SEQ ID NO: 27 - protein, 14 aa, synthetic construct
GGYSGSSYYHPMAY

SEQ ID NO: 28 - protein, 14 aa, synthetic construct
GGYSGSSYYHPMDA

SEQ ID NO: 29 - protein, 17 aa, synthetic construct
KSSQSLLASTNQKNYLA

SEQ ID NO: 30 - protein, 17 aa, synthetic construct
KSSQSLLYSTNQKNALA

SEQ ID NO: 31 - protein, 7 aa, synthetic construct
AASTRES

SEQ ID NO: 32 - protein, 9 aa, synthetic construct
QQAFIYPRT

SEQ ID NO: 33 - protein, 9 aa, synthetic construct
QQYFIAPRT

SEQ ID NO: 34 - protein, 17 aa, synthetic construct
DINPNYDTTSYSQKFKG

SEQ ID NO: 35 - protein, 12 aa, synthetic construct
SEAGNYYWYFDV

SEQ ID NO: 36 - protein, 17 aa, synthetic construct
KSSQSLLYSSNRKNYLA

SEQ ID NO: 37 - protein, 9 aa, synthetic construct
QQYYNYPYT

SEQ ID NO: 38 - protein, 5 aa, synthetic construct
NYWLG

SEQ ID NO: 39 - protein, 17 aa, synthetic construct
DIFPGSDNTYYNEKFKG

SEQ ID NO: 40 - protein, 9 aa, synthetic construct
SGNFYAMDY

SEQ ID NO: 41 - protein, 10 aa, synthetic construct
SASSSVNYMN

SEQ ID NO: 42 - protein, 7 aa, synthetic construct
DTSKLAS

SEQ ID NO: 43 - protein, 9 aa, synthetic construct
FQGSGYPFT

SEQ ID NO: 44 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 45 - protein, 123 aa, synthetic construct
EVQLVQSGAEVKKPGSSVKVSCKASGYIFTAYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 46 - protein, 123 aa, synthetic construct
EVQLVQSGAEVKKPGSSVKVSCKASGYIFTDANMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 47 - protein, 123 aa, synthetic construct
EVQLVQSGAEVKKPGSSVKVSCKASGYIFTDYNMAWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 48 - protein, 123 aa, synthetic construct
EVQLVQSGAEVKKPGSSVKVSCKASGYIFTDYNMDWVRQAPGQGLEWIGAINPDYDTTSYNEKFKGRATLTVDKSTSTAYMELSSLRSEDTAVYYCARGGYSGSSYYHPMDYWGQGTLVTVSS

SEQ ID NO: 49 - protein, 123 aa, synthetic construct
EVQLVQSGAEVKKPGSSVKVSCKASGYIFTDYNMDWVRQAPGQGLEWIGDINPAYDTTSYNEKFKGRATLTVDKSTSTAYMELSSLRSEDTAVYYCARGGYSGSSYYHPMDYWGQGTLVTVSS

SEQ ID NO: 50 - protein, 123 aa, synthetic construct
EVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSS

SEQ ID NO: 51 - protein, 123 aa, synthetic construct
EVQLVQSGAEVKKPGSSVKVSCKASGYIFTDYNMDWVRQAPGQGLEWIGDINPDYATTSYNEKFKGRATLTVDKSTSTAYMELSSLRSEDTAVYYCARGGYSGSSYYHPMDYWGQGTLVTVSS

SEQ ID NO: 52 - protein, 123 aa, synthetic construct
EVQLVQSGAEVKKPGSSVKVSCKASGYIFTDYNMDWVRQAPGQGLEWIGDINPDYDTTSANEKFKGRATLTVDKSTSTAYMELSSLRSEDTAVYYCARGGYSGSSYYHPMDYWGQGTLVTVSS

SEQ ID NO: 53 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGASGSSYYHPMDYWGQGTLVTVSS

SEQ ID NO: 54 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSAYHPMDYWGQGTLVTVSS

SEQ ID NO: 55 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYAHPMDYWGQGTLVTVSS

SEQ ID NO: 56 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMAYWGQGTLVTVSS

SEQ ID NO: 57 - protein, 123 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDAWGQGTLVTVSS

SEQ ID NO: 58 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 59 - protein, 113 aa, synthetic construct
DIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGQGTKVEIK

SEQ ID NO: 60 - protein, 113 aa, synthetic construct
DIQMTQSPSSVSASVGDRVTITCKSSQSLLYSTNQKNALAWYQQKPGKAPKLLIYWASTRESGVPSRFSGSGSGTDFTLTISSLQPEDFATYYCQQYFIYPRTFGQGTKVEIK

SEQ ID NO: 61 - protein, 113 aa, synthetic construct
DIQMTQSPSSVSASVGDRVTITCKSSQSLLYSTNQKNYLAWYQQKPGKAPKLLIAWASTRESGVPSRFSGSGSGTDFTLTISSLQPEDFATYYCQQYFIYPRTFGQGTKVEIK

SEQ ID NO: 62 - protein, 113 aa, synthetic construct
DIQMTQSPSSVSASVGDRVTITCKSSQSLLYSTNQKNYLAWYQQKPGKAPKLLIYAASTRESGVPSRFSGSGSGTDFTLTISSLQPEDFATYYCQQYFIYPRTFGQGTKVEIK

SEQ ID NO: 63 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQAFIYPRTFGQGTKVEIK

SEQ ID NO: 64 - protein, 113 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIAPRTFGQGTKVEIK

SEQ ID NO: 65 - protein, 123 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSS

SEQ ID NO: 66 - protein, 113 aa, synthetic construct
DIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIK

SEQ ID NO: 67 - protein, 121 aa, synthetic construct
EVQLQQFGAELVKPGTSVKISCKASGYTFTDYNMDWVKQGHGKGLEWIGDINPNYDTTSYSQKFKGKATLTVDKSSSTAYMELRSLTSEDTAVYYCARSEAGNYYWYFDVWGAGTTVTVSS

SEQ ID NO: 68 - protein, 113 aa, synthetic construct
DIVMSQSPSSLAVSVGEKVTMSCKSSQSLLYSSNRKNYLAWYRQKPGQSPKLLIYWASTRESGVPDRFTGSGSGTDFTLTISSVKAEDLAVYSCQQYYNYPYTFGGGTKLEIK

SEQ ID NO: 69 - protein, 118 aa, synthetic construct
QVQLQQSGAELVRPGTSVKISCKASGYAFTNYWLGWVKQRPGHGLEWIGDIFPGSDNTYYNEKFKGKATLTADKSSSTAYMQLSSLASEDSAVYFCARSGNFYAMDYWGQGTSVTVSS

SEQ ID NO: 70 - protein, 106 aa, synthetic construct
ENVLTQSPAIMSASPGEKVTMTCSASSSVNYMNWYQQKSSTFPKLWIYDTSKLASGVPGRFSGSGSGKFYSLTISSMEAEDVATYYCFQGSGYPFTFGSGTKLEIK

SEQ ID NO: 71 - protein, 703 aa, synthetic construct
HHHHHHHHHHGGSQYIKANSKFIGITELGGSCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 72 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 73 - protein, 697 aa, synthetic construct
HHHHHHHHHHSGGGGSGGGGSGGGGSKGVEPKTESERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 74 - protein, 689 aa, synthetic construct
HHHHHHHHHHENLYFQPCKGVEPKTECERLAGTESPVREEPGEDFPAARRLYWDDLKRKLSEKLDSTDFTSTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 75 - protein, 694 aa, synthetic construct
HHHHHHHHHHALEVLFQGPCKRVEQKEECVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 76 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 77 - protein, 700 aa, synthetic construct
HHHHHHHHHHSGGGGSGGGGSGGGGSKRVEQKEESVKLAETEETDKSETMETEDVPTSSRLYWADLKTLLSEKLNSIEFADTIKQLSQNTYTPREAGSQKDESLAYYIENQFHEFKFSKVWRDEHYVKIQVKSSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKERRILNIFGVIKGYEEPDRYVVVGAQRDALGAGVAAKSSVGTGLLLKLAQVFSDMISKDGFRPSRSIIFASWTAGDFGAVGATEWLEGYLSSLHLKAFTYINLDKVVLGTSNFKVSASPLLYTLMGKIMQDVKHPVDGKSLYRDSNWISKVEKLSFDNAAYPFLAYSGIPAVSFCFCEDADYPYLGTRLDTYEALTQKVPQLNQMVRTAAEVAGQLIIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 78 - protein, 697 aa, synthetic construct
HHHHHHHHHHSGGGGSGGGGSGGGGSKGVEPKTESERLAGTESPVREEPGEDFPAARRLYWDDLKRKLSEKLDSTDFTGTIKLLNENSYVPREAGSQKDENLALYVENQFREFKLSKVWRDQHFVKIQVKDSIGQNMVTIVQSNGNLDPVESPEGYVAFSKPTEVSGKLVHANFGTKKDFEELSYSVNGSLVIVRAGEITFAEKVANAQSFNAIGVLIYMDKNKFPVVEADLALFGHAHLGTGDPYTPGFPSFNHTQFPPSQSSGLPNIPVQTISRAAAEKLFGKMEGSCPARWNIDSSCKLELSQNQNVKLIVKNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYEMYNSKLLSFMKDLNQFKTDIRDMGLSLQWLYSARGDYFRATSRLTTDFHNAEKTNRFVMREINDRIMKVEYHFLSPYVSPRESPFRHIFWGSGSHTLSALVENLKLRQKNITAFNETLFRNQLALATWTIQGVANALSGDIWNIDNEF

SEQ ID NO: 79 - protein, 251 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSSGGGGSGGGGSGGGGSDIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIK

SEQ ID NO: 80 - protein, 251 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 81 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 83 - protein, 483 aa, synthetic construct
EVQLQQFGVELVKPGASVKISCKASGYIFTDYNMDWVKQSHGKSLEWIGDINPDYDTTSYNEKFKGKATLTVDKSSSTAYMELRSLTSEDTAVYYCARGGYSGSSYYHPMDYWGQGTSVTVSSGGGGSGGGGSGGGGSDIVMSQSPSSLAVSVGEKITMSCKSSQSLLYSTNQKNYLAWYQQKPGQSPELLIYWASTRESGVPDRFTGSGSGTDFTLTISNVRAEDLAVYYCQQYFIYPRTFGGGTKLEIKGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 483 aa, synthetic construct
EVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 86 - protein, 664 aa, synthetic construct
HHHHHHHHHHDYKDDDDKENLYFQGLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 87 - protein, 664 aa, synthetic construct
HHHHHHHHHHDYKDDDDKENLYFQGLYWDDLKRKLSEKLDTTDFTSTIKLLNENLYVPREAGSQKDENLALYIENQFREFKLSKVWRDQHFVKIQVKDSAQNSVIIVDKNGGLVYLVENPGGYVAYSKAATVTGKLVHANFGTKKDFEDLDSPVNGSIVIVRAGKITFAEKVANAESLNAIGVLIYMDQTKFPIVKADLSFFGHAHLGTGDPYTPGFPSFNHTQFPPSQSSGLPNIPVQTISRAAAEKLFGNMEGDCPSDWKTDSTCKMVTSENKSVKLTVSNVLKETKILNIFGVIKGFVEPDHYVVVGAQRDAWGPGAAKSSVGTALLLKLAQMFSDMVLKDGFQPSRSIIFASWSAGDFGSVGATEWLEGYLSSLHLKAFTYINLDKAVLGTSNFKVSASPLLYTLIEKTMQDVKHPVTGRSLYQDSNWASKVEKLTLDNAAFPFLAYSGIPAVSFCFCEDTDYPYLGTTMDTYKELVERIPELNKVARAAAEVAGQFVIKLTHDTELNLDYERYNSQLLLFLRDLNQYRADVKEMGLSLQWLYSARGDFFRATSRLTTDFRNAEKRDKFVMKKLNDRVMRVEYYFLSPYVSPKESPFRHVFWGSGSHTLSALLESLKLRRQNNSAFNETLFRNQLALATWTIQGAANALSGDVWDIDNEF

SEQ ID NO: 88 - protein, 251 aa, synthetic construct
EVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 89 - protein, 251 aa, synthetic construct
DIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSS

SEQ ID NO: 90 - protein, 714 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 724 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 724 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 729 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 739 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 714 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 724 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 724 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 729 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 739 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDADTTSYNEKFKGRATLTVDKSTSTAYMELSSLRSEDTAVYYCARGGYSGSSYYHPMDYWGQGTLVTVSSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 454 aa, synthetic construct
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 723 aa, synthetic construct
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIK

SEQ ID NO: 106 - protein, 723 aa, synthetic construct
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSS

SEQ ID NO: 107 - protein, 454 aa, synthetic construct
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 108 - protein, 219 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 15 aa, synthetic construct
GGGGSGGGGSGGGGS

SEQ ID NO: 110 - protein, 454 aa, synthetic construct
EVKLMESGGGLVQPGGSRKLSCAASGFTFSSFGMHWVRQAPEKGLEWVAYISSGSSTIYYGDTVKGRFTISRDNPKNTLFLQMTSLRSEDTAMYYCAREGGYYYGRSYYTMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLASTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQAFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 708 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGNSLTLSCVASGFTFSNYGMHWIRQAPKKGLEWIAMIYYDSSKMNYADTVKGRFTISRDNSKNTLYLEMNSLRSEDTAMYYCAVPTSHYVVDVWGQGVSVTVSSGGGGSGGGGSGGGGSDIQMTQSPASLSASLEEIVTITCQASQDIGNWLAWYQQKPGKSPQLLIYGATSLADGVPSRFSGSRSGTQFSLKISRVQVEDIGIYYCLQAYNTPWTFGGGTKLELKGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 706 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVQSGAEVKKPGASVKVSCKASGYTFTSYWMHWVRQAPGQRLEWIGEINPTNGRTNYIEKFKSRATLTVDKSASTAYMELSSLRSEDTAVYYCARGTRAYHYWGQGTMVTVSSGGGGSGGGGSGGGGSDIQMTQSPSSLSASVGDRVTITCRASDNLYSNLAWYQQKPGKSPKLLVYDATNLADGVPSRFSGSGSGTDYTLTISSLQPEDFATYYCQHFWGTPLTFGQGTKVEIKGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 724 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 739 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 724 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 719 aa, synthetic construct
DIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSEVQLVQSGAEVKKPGSSVKVSCKASGYIFTDYNMDWVRQAPGQGLEWIGDINPDYDTTSYNEKFKGRATLTVDKSTSTAYMELSSLRSEDTAVYYCARGGYSGSSYYHPMDYWGQGTLVTVSSGGGGSGGGGSGGGGSDIQMTQSPSSVSASVGDRVTITCKSSQSLLYSTNQKNYLAWYQQKPGKAPKLLIYWASTRESGVPSRFSGSGSGTDFTLTISSLQPEDFATYYCQQYFIYPRTFGQGTKVEIKGGGGSGGGGSGGGGSGGGGSDIVMTQAPLSLPVSLGDQASISCRSSQSIVHSNGNTYLEWYLQKPGQSPKLLIYKVSNRFSGVPDRFSGSGSGTDFTLKISRVEAEDLGIYYCFQGSHVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0274980 A1
(43) Pub. Date: Sep. 17, 2026

(54) ANTIBODY BINDING TO THE PROTEASE-LIKE DOMAIN OF THE HUMAN TRANSFERRIN RECEPTOR HTFR1

(71) Applicant: BioArctic AB, Stockholm (SE)

(72) Inventors: Ronny Falk, Stockholm (SE); Ken Honek, Stockholm (SE); Martin Larhammar, Stockholm (SE); Hanna Laudon, Stockholm (SE); Lisa Sandersjöö, Stockholm (SE)

(21) Appl. No.: 19/167,480

(22) PCT Filed: Mar. 22, 2024
(86) PCT No.: PCT/EP2024/057780

§ 371 Date: Sep. 22, 2025

(30) Foreign Application Priority Data
Mar. 24, 2023 (EP) 23164089.7
Mar. 24, 2023 (EP) 23164092.1

(51) Int. Cl. C07K 16/28; A61K 39/00; C07K 16/18

(52) U.S. Cl. CPC C07K 16/2881; C07K 16/18; A61K 2039/505; C07K 2317/31; C07K 2317/565; C07K 2317/622; C07K 2317/732; C07K 2317/734

(57) ABSTRACT

The present disclosure relates to a binding molecule, for example an antibody or antigen-binding fragment thereof, which binds to the protease-like domain of human transferrin receptor 1 (TfR1), and to therapeutic and diagnostic uses thereof.

### FIELD

The present disclosure relates to a binding molecule, for example an antibody or antigen-binding fragment thereof, which binds to the protease-like domain of human transferrin receptor 1 (TfR1), and to therapeutic and diagnostic uses thereof.

### BACKGROUND

Treatment modalities for brain and neurological diseases are limited, due to the impermeability of the blood vessels of the brain to most substances carried in the bloodstream (Freskgard and Urich (2017), Neuropharmacology 120:38-55; Stanimirovic et al (2018), BioDrugs 32:547-559). The small blood vessels (capillaries) of the brain, referred to collectively as the blood-brain barrier (BBB), are unique when compared to the blood vessels found in the periphery of the body. Tight apposition of BBB endothelial cells (EC) to neural cells, such as astrocytes, pericytes and neurons, induces phenotypic features that contribute to the observed impermeability. Tight junctions between ECs in the BBB limit paracellular transport, while the lack of passive pinocytotic vesicles and fenestrae limit non-specific transcellular transport. These factors combine to restrict molecular flux from the blood to the brain in general to molecules that are less than 500 Da in size and lipophilic. Thus, the otherwise promising prospect of using the large mass transfer surface area (over 20 m² from 600 km of capillaries in a human brain) of the blood stream as a delivery vehicle is made largely infeasible, except in those circumstances where a drug with the desired pharmacological properties fortuitously possesses size and lipophilicity attributes which allow it to pass through the BBB. Because of such restrictions, it has been estimated that more than 98% of all small molecule pharmaceuticals and nearly 100% of the emerging class of protein and gene therapeutics do not cross the BBB.

WO91/03259 proposes a principle for transporting a neuropharmaceutical agent across the BBB, which involves conjugating the agent to an antibody which is reactive with the transferrin receptor. According to this disclosure, binding of the conjugate to the transferrin receptor leads to active transport of the conjugate across the BBB. Later work has developed this concept further, for example as described in WO2012/075037, WO2014/033074, WO2018/011353 and WO2022/258841, all describing different formats for achieving transport of a biopharmaceutical agent across the BBB by utilizing the transferrin receptor.

There exist two forms of the human transferrin receptor. Transferrin receptor 1 (TfR1) is the target for the binding molecule of the present disclosure. TfR1 is an iron transporter protein, which maintains cellular iron levels by recognizing and internalizing through specific binding of the iron carrier proteins transferrin (Tf) and ferritin (Ft) into cells through endocytosis mediated by clathrin-coated vesicles. TfR1 is expressed in numerous cells and organs, but expression levels vary and, importantly, TfR1 is expressed to a higher degree on BBB endothelial cells than on other endothelial cells, making the receptor a target for neuropharmaceutical delivery. Structurally, TfR1 is a dimeric transmembrane glycoprotein comprising the amino acid sequence SEQ ID NO:85, which has a large ectodomain (residues 89-760), an intramembrane region (residues 62-88) and a cytoplasmic domain (residues 1-61). The ectodomain in turn has three distinct domains held separate from the cell surface by a stalk region (residues 89-120). These three parts of the ectodomain are the helical domain (residues 606-760), the protease-like domain (residues 121-183, 384-605) and the apical domain (residues 184-383) (Lawrence et al (1999), Science 286:779-782).

In the context of BBB transport via the TfR1, antibodies and fragments thereof which have affinity for TfR1 have been described. By way of example, a number of TfR1 binding antibodies are disclosed in WO2014/189973, in which antibodies are grouped according to epitope specificity in classes I-IV (see e.g. FIG. 3 and the associated figure description on page 30 lines 11-15). Classes I-III of WO2014/189973 are denoted “apical binders” whereas the antibody of class IV is denoted a “non-apical binder”. Other TfR1 binding antibodies are disclosed in EP3088518, EP3315606 and EP3560958, however without any information about the epitope specificity of these disclosed antibodies.

Thus, most work on using TfR1 as a target for binding and BBB transport has focused on apical binders. This is thought to be because the apical domain is the structure within TfR1 that seems to provoke a strong immune response and thus to trigger antibody generation in animals when used as an immunogen. Thus, most known antibody binders against TfR1 have epitopes that are located within the apical domain. Another indication that the apical domain contains structures prone to engage with various ligands is that viruses have been described to utilize epitopes within the apical domain to enter cells (Cohen-Dvashi et al (2020), Nat Commun 11:67).

Furthermore, the detailed structure of the TfR1 and ferritin complex was recently determined (Montemiglio et al (2019), Nat Commun 10:1121), showing that the interface between TfR1 and ferritin is located within the apical domain. This suggest that TfR1 apical binders could potentially interfere with the binding of ferritin to TfR1 if used for BBB transport and in this way influence the normal function of ferritin in iron transport. Also, the binding and uptake of H-ferritin have been shown to be mediated by TfR1 (Li et al (2010), Proc Natl Acad Sci USA 107(8):3505-10). Thus, there are reasons to conclude that binders directed against the apical domain of TfR1, and especially binding to the binding site used by ferritin, may negatively influence the important function of ferritin in transporting iron via the binding to TfR1.

It has been reported that TfR1 apical binders can induce both acute clinical signs and decreased in circulating reticulocytes (Couch et al (2013), Sci Transl Med 5:183ra57). The TfR1 has also been described in relation to anemia and iron deficiency (Braga et al (2014), Clin Chim Acta 431:143-147). Anemia due to autoantibodies to TfR1 has also been described (Hyman et al (1984), N Engl J Med 311:214-218). Taken together, the data suggest that TfR1 binding and interfering with iron transporters such as transferrin and/or ferritin could lead to safety issues such as reduction in reticulocyte levels and anemia.

To date, the focus within the field has been to avoid interfering with one of the described TfR1 ligands, namely transferrin. This has guided the field to utilize binding sites in the apical domain of TfR1, distant from the binding site of transferrin. However, such apical binders may still interfere with the other important TfR1 ligand, ferritin, leading to interference in iron transport and function.

Against this background, there remains a need in the field for antibodies and other binding molecules, which have a binding affinity for TfR1 but which do not exhibit the drawbacks and risks associated with hitherto known binding molecules.

### DISCLOSURE OF THE INVENTION

It is an object of the disclosure to address this need, by providing a TfR1 binding molecule which utilizes a different binding site on TfR1 than the naturally occurring ligands.

One such object is to provide a TfR1 binding molecule which utilizes a different binding site on TfR1 than transferrin.

Another such object is to provide a TfR1 binding molecule which utilizes a different binding site on TfR1 than ferritin.

Yet another such object is to provide a TfR1 binding molecule which utilizes a different binding site on TfR1 than HFE (homeostatic iron regulator).

A related object of the disclosure is to provide a TfR1 binding molecule which interacts with TfR1 in a way which minimizes the interference with TfR1 itself and/or its normal function.

Another object of the disclosure is to provide a TfR1 binding molecule suitable for use as a fusion partner in constructs arranged for transport through the BBB.

One or more of these objects, and other objects that are evident to the skilled person from the teachings herein, are met by the various aspects of the disclosure.

Thus, in a first aspect, the present disclosure provides a transferrin receptor 1 (TfR1) binding molecule, capable of selective binding to an epitope located in the protease-like domain of TfR1 defined by amino acid residues 121-183 and 384-605 in SEQ ID NO:85. Without wishing to be bound by theory, the binding to TfR1 to an epitope, or binding site, within the protease-like domain is contemplated to offer advantages in terms of avoiding the drawbacks associated with known binders to TfR1, in particular those known binders which have affinity for epitopes or binding sites located in the apical domain of TfR1.

In a specific embodiment, the epitope or binding site for the TfR1 binding molecule of the disclosure comprises the amino acid residues 150, 151, 154, 158, 159, 161, 163 and 385 in SEQ ID NO:85. In another embodiment, the epitope or binding site for the TfR1 binding molecule of the disclosure consists of the amino acid residues 150, 151, 154, 158, 159, 161, 163 and 385 in SEQ ID NO:85. In an alternative specific embodiment, the epitope or binding site for the TfR1 binding molecules comprises or consists of at least one, at least two, at least three, at least four, at least five, at least six, at least seven or all eight of the amino acid residues 150, 151, 154, 158, 159, 161, 163 and 385 in SEQ ID NO:85. As shown in the examples which follow, for example with reference to FIG. 12, this embodiment of the epitope for the binding molecules identified and disclosed herein ensures binding that does not interfere with the natural TfR1 ligands transferrin and ferritin.

As known to a person skilled in the art, an epitope (or “antigenic determinant”) is a group of amino acids or other chemical groups exposed on the surface of a molecule, frequently a protein, here hTfR1, which can generate an antigenic response and bind antibody. An epitope is a localized region on the surface of an antigen that is recognized by the immune system, specifically by antibodies. A conformational epitope is composed of neighboring amino acid residues located on an antigenic protein surface structure. Conformational epitopes bind their complementary paratopes in B-cell receptors and/or antibodies. In one embodiment of the disclosure, the epitope bound by the binding molecule is a conformational epitope.

In one embodiment of the binding molecule, it comprises an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope. Said VH/VL pair may for example be provided in a full-length traditional antibody, or in an antibody fragment selected from the group consisting of a Fab fragment, a single chain Fab (scFab) fragment, an Fv fragment and a single chain (scFv) fragment. In a specific embodiment, the VH/VL pair forms part of an scFv. The designation of “VH/VL” as used in relation to a VH/VL pair does not limit the construct to any particular order of the VH and VL regions in the polypeptide chain, but is only used to convey that both the VH and VL regions are present, and that they are capable of pairwise association to form an Ig domain with an antigen-binding surface. As such, the term “VH/VL pair” encompasses, for example, constructs in which the VL region precedes the VH region in a single chain Fv, constructs in which the VH region precedes the VL region in a single chain Fv, and constructs in which the VH and VL regions are non-covalently associated with each other.

With respect to the antigen-binding surface of the VH/VL pair, it may suitably be composed of three complementarity-determining regions (CDRs) from said VH region and three CDRs from said VL region. In one embodiment, said CDRs comprise the following:

VHCDR1:
(SEQ ID NO: 1)
X1X2NMX3,

wherein
X1 is selected from D and A;
X2 is selected from Y and A; and
X3 is selected from D and A;

VHCDR2:
(SEQ ID NO: 2)
X4INPX5X6X7TTSX8X9X10KFKG,

wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;

VLCDR1:
(SEQ ID NO: 4)
KSSQSLLX11SX12NX13KNX14LA,

wherein
X11 is selected from Y and A;
X12 is selected from T and S;
X13 is selected from Q and R; and
X14 is selected from Y and A;

VLCDR2:
(SEQ ID NO: 5)
X15ASTRES

wherein
X15 is selected from W and A; and

VLCDR3:
(SEQ ID NO: 6)
QQX16X17X18X19PX20T

wherein
X16 is selected from Y and A;
X17 is selected from F and Y;
X18 is selected from I and N;
X19 is selected from Y and A; and
X20 is selected from R and Y.

In one embodiment, the CDRs further comprise:

VHCDR3:
(SEQ ID NO: 3)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

In an alternative embodiment, the CDRs further comprise:

VHCDR3:
(SEQ ID NO: 35)
SEAGNYYWYFDV

As defined herein, embodiments of the binding molecule of the first aspect of the disclosure that comprise a VH/VL pair have specific amino acid sequences in the regions determining its binding capability, such as the CDRs of the heavy and/or light chain variable domain, or indeed the entire VL and/or VH domains or regions. Non-limiting examples of such specific amino acid sequences are provided herein for the specific antibodies generated and characterized as described in Examples 1-9. It is contemplated that the specific sequence information provided for the generated antibodies enables the skilled person to define combinations and variations of these sequences within the scope of the invention, such as including the combinations and variations afforded by the variation in the general CDR sequences provided as SEQ ID NO:1-6.

In one embodiment, said CDRs further comprise

VHCDR3:
(SEQ ID NO: 3)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

In one embodiment, said VHCDR2 is:

VHCDR2:
(SEQ ID NO: 7)
X4INPX5X6X7TTSX8NEKFKG,

wherein
X4 is selected from D and A;
X5 is selected from D and A;
X6 is selected from Y and A;
X7 is selected from D and A; and
X8 is selected from Y and A.

In one embodiment, said VLCDR1 is:

VLCDR1:
(SEQ ID NO: 8)
KSSQSLLX11STNQKNX14LA,

wherein
X11 is selected from Y and A; and
X14 is selected from Y and A.

In one embodiment, said VLCDR3 is:

VLCDR3:
(SEQ ID NO: 9)
QQX16FIX19PRT

wherein
X16 is selected from Y and A;
X19 is selected from Y and A.

In one embodiment, the amino acid sequence of said VHCDR1 is selected from the group consisting of SEQ ID NO:10 and 16-18.

In one embodiment, the amino acid sequence of said VHCDR2 is selected from the group consisting of SEQ ID NO:11, 19-23 and 34, for example selected from the group consisting of SEQ ID NO:11 and 19-23.

In one embodiment, the amino acid sequence of said VHCDR3 is selected from the group consisting of SEQ ID NO:12, 24-28 and 35, for example selected from the group consisting of SEQ ID NO:12 and 24-28.

In one embodiment, the amino acid sequence of said VLCDR1 is selected from the group consisting of SEQ ID NO:13, 29, 30 and 36, for example selected from the group consisting of SEQ ID NO:13, 29 and 30.

In one embodiment, the amino acid sequence of said VLCDR2 is selected from the group consisting of SEQ ID NO:14 and 31.

In one embodiment, the amino acid sequence of said VLCDR3 is selected from the group consisting of SEQ ID NO:15, 32, 33 and 37, for example selected from the group consisting of SEQ ID NO:15, 32 and 33.

In some embodiments, the CDR sequences can be freely combined among the options listed above. Such embodiments for example include, but are not limited to, those combinations exemplified in Example 9 for alanine substituted variants of the h26D3 embodiment of the binding molecule of the disclosure.

In a specific embodiment of a binding molecule of the disclosure comprising a VH/VL pair, the amino acid sequences of the six CDRs are the following:

VHCDR1:
(SEQ ID NO: 10)
DYNMD,
VHCDR2:
(SEQ ID NO: 11)
DINPDYDTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

In another specific embodiment of a binding molecule of the disclosure comprising a VH/VL pair, the amino acid sequences of the six CDRs are the following:

VHCDR1:
(SEQ ID NO: 10)
DYNMD,
VHCDR2:
(SEQ ID NO: 21)
DINPDADTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

In another specific embodiment of a binding molecule of the disclosure comprising a VH/VL pair, the amino acid sequences of the six CDRs are the following:

VHCDR1:
(SEQ ID NO: 10)
DYNMD,
VHCDR2:
(SEQ ID NO: 34)
DINPNYDTTSYSQKFKG,
VHCDR3:
(SEQ ID NO: 35)
SEAGNYYWYFDV
VLCDR1:
(SEQ ID NO: 36)
KSSQSLLYSSNRKNYLA,
VLCDR2:
(SEQ ID NO: 14)
WASTRES
VLCDR3:
(SEQ ID NO: 37)
QQYYNYPYT

In another specific embodiment of a binding molecule of the disclosure comprising a VH/VL pair, the amino acid sequences of the six CDRs are the following:

VHCDR1:
(SEQ ID NO: 38)
NYWLG,
VHCDR2:
(SEQ ID NO: 39)
DIFPGSDNTYYNEKFKG,
VHCDR3:
(SEQ ID NO: 40)
SGNFYAMDY
VLCDR1:
(SEQ ID NO: 41)
SASSSVNYMN,
VLCDR2:
(SEQ ID NO: 42)
DTSKLAS
VLCDR3:
(SEQ ID NO: 43)
FQGSGYPFT

In one embodiment, CDR sequences in an antigen-binding interface comprised in a binding molecule of the disclosure are as defined using the Kabat convention, which is well known to a person of skill in the art of antibody technology (see e.g. Kabat (1991), Sequences of Proteins of Immunological Interest, 5^{th} edition, NIH Publication no 91-3242 from the US Department of Health and Human Services).

In one embodiment of a binding molecule of the disclosure comprising a VH/VL pair, said VH region comprises or consists of an amino acid sequence selected from
(i) the group consisting of SEQ ID NO:44-57, 65 and 67, for example the group consisting of SEQ ID NO:44-57, for example the group consisting of SEQ ID NO:44 and 50; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

In one embodiment of a binding molecule of the disclosure comprising a VH/VL pair, said VL region comprises or consists of an amino acid sequence selected from
(i) the group consisting of SEQ ID NO:58-64, 66 and 68, for example the group consisting of SEQ ID NO:58-64; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

In a particular such embodiment, the VH region and VL region are both as defined immediately above, i.e. a VH comprising or consisting of a sequence selected from SEQ ID NO:44-57 and sequences having at least 80% sequence identity thereto, and a VL comprising or consisting of a sequence selected from SEQ ID NO:58-64 and sequences having at least 80% sequence identity thereto.

In another embodiment of a binding molecule of the disclosure comprising a VH/VL pair, said VH region comprises or consists of an amino acid sequence selected from SEQ ID NO:69 and a sequence having at least 80%, at least 90%, at least 92%, at least 94%, at least 95%, at least 96%, at least 97%, at least 98% or at least 99% identity to SEQ ID NO:69, provided that the sequences of the CDR regions are 100% identical to those of SEQ ID NO:69.

In another embodiment of a binding molecule of the disclosure comprising a VH/VL pair, said VL region comprises or consists of an amino acid sequence selected from SEQ ID NO:70 and a sequence having at least 80%, at least 90%, at least 92%, at least 94%, at least 95%, at least 96%, at least 97%, at least 98% or at least 99% identity to SEQ ID NO:70, provided that the sequences of the CDR regions are 100% identical to those of SEQ ID NO:70.

In a particular such embodiment, the VH region and VL region are both as defined immediately above, i.e. a VH comprising or consisting of the sequence SEQ ID NO:69 and sequences having at least 80% sequence identity thereto, and a VL comprising or consisting of the sequence SEQ ID NO:70 and sequences having at least 80% sequence identity thereto.

In one embodiment of a binding molecule of the disclosure comprising a VH/VL pair, said VH region comprises SEQ ID NO:44 and said VL region comprises a sequence selected from SEQ ID NO:58-64.

In one embodiment of a binding molecule of the disclosure comprising a VH/VL pair, said VH region comprises a sequence selected from SEQ ID NO:44-57 and said VL region comprises SEQ ID NO:58.

In one embodiment of a binding molecule of the disclosure comprising a VH/VL pair, said VH region comprises SEQ ID NO:44 and said VL region comprises SEQ ID NO:58.

In one embodiment of a binding molecule of the disclosure comprising a VH/VL pair, said VH region comprises SEQ ID NO:50 and said VL region comprises SEQ ID NO:58.

In certain embodiments, the VH and VL sequences, when present in the binding molecule, are selected from any one of the listed sequences and sequences having at least 80%, at least 90%, at least 92%, at least 94%, at least 95%, at least 96%, at least 97%, at least 98%, at least 99% or at least 100% identity thereto.

In one embodiment of a binding molecule of the disclosure comprising a VH/VL pair, said VH/VL pair forms part of an antibody construct.

In one such embodiment, said antibody construct has more than one binding specificity. It may for example be bispecific or trispecific, or have more than three binding specificities. In a particular embodiment, the binding molecule is bispecific, for example a bispecific antibody construct.

In one embodiment of a binding molecule of the disclosure comprising a VH/VL pair, the VH/VL pair is present in an antibody fragment selected from the group consisting of a Fab fragment, a single chain Fab (scFab) fragment, an Fv fragment and a single chain (scFv) fragment. In a particular embodiment of such a binding molecule, said antibody fragment is an scFv.

In an important embodiment of the binding molecule disclosed herein, it further comprises an antibody or an antigen binding fragment thereof, in addition to the VH/VL pair providing selective binding to hTfR1. In one such embodiment, this additional antibody or fragment thereof is capable of selective binding to a target present in the brain of a mammal. In some embodiments, said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-3 precursor protein, p75 neurotrophin receptor, neuregulin and caspase 6. In a more specific embodiment, said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), Tau, phosphorylated Tau or fragments thereof and apolipoprotein E4. In an even more specific embodiment, said target is selected from the group consisting of amyloid-β peptide or derivatives or fragments thereof, alpha-synuclein or derivatives or fragments thereof and TAR DNA-binding protein 43 (TDP-43) or derivatives or fragments thereof.

In one embodiment, said antibody or antigen binding fragment thereof capable of selective binding to a target present in the brain of a mammal is an anti-AR antibody, for example an antibody selected from the group consisting of lecanemab, gantenerumab, aducanumab, donanemab, PBD-C06 and KHK6640.

In another embodiment, said antibody or antigen binding fragment thereof capable of selective binding to a target present in the brain of a mammal is an anti-alpha-synuclein antibody, for example an antibody selected from the group consisting of prasinezumab, UCB7853, Lu AF82422, TAK-341 and BAN0805.

### Affinity for a Target

As used herein, the terms “specific binding to X”, “selective binding to X” and “affinity for X”, wherein X is a target (e.g. an antigen or an epitope, such as the TfR1 bound by the VH/VL pair of the binding molecule as defined above), refer to a property of a binding molecule, such as a property of an antibody or antigen-binding fragment thereof or of a bi- or multispecific construct incorporating such an antibody or antigen-binding fragment thereof, which may be tested for example by ELISA, by surface plasmon resonance (SPR) or by bio-layer interferometry (BLI). The skilled person is aware of these methods and others.

For example, the binding affinity for a target, antigen or epitope X may be tested in an experiment in which a binding molecule to be tested is captured on ELISA plates coated with X or a molecule comprising the epitope X, and a biotinylated detector antibody is added, followed by streptavidin-conjugated horse radish peroxidase (HRP). Alternatively, said detector antibody may be directly conjugated with HRP. Tetramethylbenzidine (TMB) substrate is added and the absorbance at 450 nm is measured using an ELISA multi-well plate reader. The skilled person may then interpret the results obtained by such experiments to establish at least a qualitative measure of the binding affinity for X of the binding molecule. If a quantitative measure is desired, for example to determine the EC50 value (the half maximal effective concentration) for the interaction, ELISA may also be used. The response of the binding molecule against a dilution series of X may be measured using ELISA as described above. The skilled person may then interpret the results obtained by such experiments and EC50 values may be calculated from the results, using for example GraphPad Prism v.9 and non-linear regression.

As used herein, the term “EC50” refers to the half maximal effective concentration of binding molecule which induces a response halfway between the baseline and maximum after a specified exposure time.

Additionally or alternatively, inhibition ELISA may be used to obtain a quantitative measure of interaction by determination of the “IC50” (the half maximal inhibitory concentration). In an inhibition ELISA, the concentration of target X in a fluid sample is measured by detecting interference in an expected signal output. In principle, a known target or epitope-bearing substance is used to coat a multi-well plate. In parallel, a binding molecule with putative affinity for X is added and incubated with a solution containing target at varied concentrations. Following standard blocking and washing steps, samples containing the mixture of said binding molecule and the target are added to the well. Labeled detection antibody with affinity for the binding molecule is then applied for detection using relevant substrates (for example TMB). In principle, if there is a high concentration of target in the fluid sample, a significant reduction in signal output will be observed. In contrast, if there is very little target in the fluid sample, there will be very little reduction in the expected signal output. The skilled person appreciates that the signal output is also dependent on the affinity of the binding molecule for said target.

As used herein, the term “IC50” refers to the half maximal inhibitory concentration of a binding molecule which induces a response halfway between the baseline and maximum inhibition after a specified exposure time. Herein, a lower IC50 value indicates that a lower concentration of target is required to interfere with the binding of the detection antibody to the known target coated on the plate, as compared to a higher IC50 value. Thus, a lower IC50 value typically corresponds to a higher affinity.

The binding affinity of a binding molecule may also be tested by surface plasmon resonance (SPR). For example, the affinity may be tested in an experiment in which target or epitope X is immobilized on a sensor chip of the instrument, and the sample containing the binding molecule to be tested is passed over the chip. Alternatively, the binding molecule to be tested may be immobilized on a sensor chip of the instrument, and a sample containing X is passed over the chip. The skilled person may then interpret the results obtained by such experiments to establish at least a qualitative measure of the binding affinity for X of the binding molecule. If a quantitative measure is desired, for example to determine a K_{D} value for the interaction, SPR may also be used. Binding values may for example be defined in a Biacore (Cytiva) or ProteOn XPR 36 (Bio-Rad) instrument. The target or epitope is suitably immobilized on a sensor chip of the instrument, and samples of the binding molecule whose affinity is to be determined are prepared by serial dilution and injected. K_{D} values may then be calculated from the results using for example the 1:1 Langmuir binding model of the Biacore Insight Evaluation Software 2.0 or other suitable software, typically provided by the instrument manufacturer.

The binding affinity may also be measured by bio-layer interferometry (BLI), a label-free technology for measuring biomolecular interactions within the interactome. It is an optical analytical technique that analyzes the interference pattern of white light reflected from two surfaces: a layer of immobilized protein on the biosensor tip, and an internal reference layer. The binding between a ligand (target or epitope X) immobilized on the biosensor tip surface and an analyte (such as a binding molecule with a putative affinity for X) in solution produces an increase in optical thickness at the biosensor tip resulting in a wavelength shift, Δλ, which is a direct measure of the change in thickness of the biological layer. Interactions are measured in real time, providing the ability to monitor binding specificity, rates of association and dissociation, or concentration, with precision and accuracy.

The skilled person is aware of the above mentioned and other methods for measuring the affinity of a binding molecule for a target or epitope X, either qualitatively or quantitatively or both.

### Pharmaceutical Compositions

In a second aspect, the disclosure provides a pharmaceutical composition comprising a binding molecule as described herein and at least one pharmaceutically acceptable excipient or carrier.

Techniques for formulating polypeptides such as antibodies and their derivatives for human therapeutic use are well known in the art and are reviewed, for example, in Wang et al (2007), J Pharm Sci, 96:1-26, the contents of which are incorporated herein in their entirety.

Pharmaceutically acceptable excipients that may be used to formulate the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances (for example sodium carboxymethylcellulose), polyethylene glycol, polyacrylates, waxes, polyethylene-polyoxypropylene block polymers, polyethylene glycol and wool fat.

In certain embodiments, the pharmaceutical compositions are formulated for administration to a subject via any suitable route of administration including but not limited to intramuscular, intravenous, intradermal, intraperitoneal injection, subcutaneous, epidural, nasal, oral, rectal, topical, inhalational, buccal (e.g., sublingual), and transdermal administration. In preferred embodiments, the composition is formulated for intravenous or subcutaneous administration.

### Methods of Prevention, Treatment, Diagnosis, Prognosis and Detection

The binding molecule according to the present disclosure may be useful as a therapeutic, prophylactic, diagnostic and/or prognostic agent.

Hence, in a further aspect of the disclosure, there is provided a binding molecule according to the first aspect, or a pharmaceutical composition according to the second aspect, for use as a medicament.

In yet another aspect of the disclosure, there is provided a binding molecule according to the first aspect, or a pharmaceutical composition according to the second aspect, for use as a diagnostic agent.

In yet another aspect of the disclosure, there is provided a binding molecule according to the first aspect, or a pharmaceutical composition according to the second aspect, for use as a prognostic agent.

Also provided are methods of preventing, treating or diagnosing disease or assessing disease prognosis, wherein a binding molecule as disclosed herein is administered to a subject in need thereof, typically a human subject.

Also provided is the use of the disclosed binding molecule for the manufacture of compositions (such as medicaments) for use in the prevention, treatment, diagnosis and/or prognosis of any one of the listed diseases.

Thus, in one embodiment, the binding molecule, or pharmaceutical composition comprising it, is useful in the treatment, prevention, diagnosis and/or prognosis of a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

In a more specific embodiment, said disorder is selected from Alzheimer's disease and other disorders associated with Aβ protein aggregation, Lewy body dementia (LBD), Down's syndrome (DS), amyotrophic lateral sclerosis (ALS), frontotemporal dementia, tauopathy, Parkinson's disease (PD), Parkinson's disease dementia (PDD) and the Lewy body variant of Alzheimer's disease.

In an even more specific embodiment, said disorder is selected from Alzheimer's disease and other disorders associated with Aβ protein aggregation, Lewy body dementia (LBD), amyotrophic lateral sclerosis (ALS) and Parkinson's disease (PD), in particular Alzheimer's disease.

In an alternative embodiment, the binding molecule, or pharmaceutical composition comprising it, is useful in the treatment, prevention, diagnosis and/or prognosis of another disorder, for example a disorder selected from brain cancer, multiple sclerosis and lysosomal storage diseases.

In another aspect, there is provided a method of treatment, prevention, diagnosis and/or prognosis of a disorder as listed above, said method comprising administering to said mammal an amount, such as a therapeutically effective amount, of a binding molecule, or pharmaceutical composition comprising it.

### INCORPORATION BY REFERENCE

Various publications are cited in the present application, each of which is incorporated by reference herein in its entirety.

### BRIEF DESCRIPTION OF THE FIGURES

FIG. 1 shows the results of a binding screen of the indicated IgG antibodies from the immunization described in Example 1 towards human (hTfR1), cyno (cTfR1) and mouse (mTfR1) TfR1 in crude hybridoma supernatants by biolayer interferometry (BLI).

FIG. 2 shows the result of the BLI binding analysis described in Example 2 for the indicated Fab fragments of mouse antibodies 24B4, 26D3 and 37D10 as well as for a Fab fragment of control antibody 8D3.

FIG. 3 shows mapping of antibody binding epitopes to the protease-like domain of hTfR1 as described in Example 2, by selective antibody binding to ELISA plates coated with either human, mouse or one of three different chimeric human/mouse TfR1 receptors. Antibodies 24B4, 26D3 and 37D10 bind to hTfR1 (A) but not to mTfR1 (B). In addition, 24B4, 26D3 and 37D10 also bind to h/m protease like domain chimera (D), but not to any of the plates coated with the other chimeric receptors (C and E).

FIG. 4 illustrates the epitope binning assay described in Example 2, with the following main four steps: Step 1—immobilization of bio-TfR1 on sensor chip; Step 2—wash of non-binding material; Step 3—binding of competing binder to TfR1; Step 4—association of binders to the TfR1:binder complex formed in Step 3. The data in Step 4 determines whether the two investigated binders compete in binding to hTfR1.

FIG. 5 shows the result of carrying out the epitope binning assay as described in Example 2, showing the degree of competition between antibodies for simultaneous binding to hTfR1. Binding of (A) antibody 26D3, (B) antibody 24B4 and (C) control antibody 15G11-1 to preformed complexes of hTfR1 and either of the indicated antibodies. Binding responses for all antibodies are normalized to the binding response measured to free hTfR1 (no competing antibody).

FIG. 6 shows binding by the indicated binders to hTfR1 on the surface of cells, studied as described in Example 2. The Y axes of both diagrams show the mean fluorescence intensity when staining cells with (A) whole antibodies and (B) Fab fragments of the indicated binders. No background staining is detected with the negative isotype control IgG (A) or the non-related Fab fragment, Ly128 (B).

FIG. 7 shows the result of competition analysis of indicated binders with ferritin and transferrin as described in Example 3. The diagrams show (A) MFI of the indicated binders binding to TfR1 expressed on THP-1 cell surfaces, (B) MFI of ferritin on cell surface when exposed to the indicated binders, with the positive control antibody MA-712 competing with ferritin, and (C) MFI of transferrin on cell surfaces when exposed to the indicated binders.

FIG. 8 is a collection of sensorgrams showing the result of SPR analysis of original 26D3 and 26D3 humanized as described in Example 4 (h26D3) in Fab formats when binding to hTfR1 and cTfR1 as indicated.

FIG. 9 shows the result of BLI and ELISA binding studies carried out on mouse and humanized versions of 26D3 in an scFv format as described in Example 4. (A) Sensorgrams obtained by BLI measurement of binding of the indicated constructs to hTfR1. (B) Binding responses from ELISA measurement of binding of the indicated constructs to coated TfR1.

FIG. 10 are depictions of the x-ray structure of the complex of 26D3-Fab and hTfR1, determined as described in Example 5. The chain names as used in the coordinate files are indicated. (A) Refined structure showing overall folds of three independent complexes in the asymmetric unit. (B) Example of electron density (2m|Fo|−D|Fc|) contoured at the 16 level. The protein chains are drawn in cartoon representation while sugar moieties are shown in stick representation.

FIG. 11 is a ribbon representation of the h26D3-Fab human TfR1 complex determined with x-ray crystallography as described in Example 5. h26D3-Fab is depicted in dark gray and hTfR1 in white. The binding interface (epitope/paratope) is encircled.

FIG. 12 is a surface area representation of hTfR1 with the binding sites for the natural ligands ferritin and transferrin indicated, as well as the epitope for the binder 26D3 of the present disclosure. The different binding sites and epitope are depicted with a circle around each specific site.

FIG. 13 illustrates the work on generating and characterizing an hTfR1-KI mouse model as described in Example 6. (A) Schematic illustration of the transgenic hTfR1-KI mouse construct. The extracellular domain of human TFRC was inserted in the murine Tfrc gene by homologous recombination. (B) Quantitative reverse transcription PCR (RT-qPCR) analysis of mouse Tfrc and human TFRC gene expression in brain (N=3/genotype). hTfR1-KI mice (grey circles) express human TFRC and mouse Tfrc in total brain homogenate, WT littermates only express mouse Tfrc (white). (C) Western blot analysis for hTfR1, total TfR1, and hTfR1-KI in brain. hTfR1-KI animals at 6-8 months (N=5) and 15 months (N=4) express comparable levels of hTfR1 protein. Total TfR1 levels are comparable between hTfR1-KI transgenic and WT littermates (N=3).

FIG. 14 shows the results of in vivo brain and plasma exposure analysis of various indicated hTfR1 binding molecules in hTfR1-KI transgenic mice as described in Example 7. (A) Brain exposure 24 h after i.v. administration of the indicated hTfR1 binders. (B) Plasma exposure 24 h after i.v. administration of the indicated hTfR1 binders. (C) Brain:Plasma ratio 24 h after i.v. administration of the indicated hTfR1 binders. The negative control is denoted “158”, and the positive control “15G11-1”.

FIG. 15 shows the results of in vivo brain exposure analysis of various indicated hTfR1 binding molecules in hTfR1-KI mice by immunohistochemistry as described in Example 8. Cortical brain capillary staining observed for several binding molecules, including h26D3. Reference hTfR1-binder “15G11-1” and non-TfR1 binder “158” were used as positive and negative control, respectively.

FIG. 16 shows BLI sensorgrams for the indicated alanine variants of h26D3 as described in Example 9. Each variant showed a different kinetic profile, illustrating the possibility to generate variants with different affinity against human TfR1 with specific mutations in the CDR regions of the heavy or light chain.

FIG. 17 shows representative SPR sensorgrams of the interaction between the indicated alanine variants of h26D3 with hTfR1 and cTfR1, measured as described in Example 9.

FIG. 18 shows the results of indirect ELISA analysis of the binding of the indicated alanine variants of h26D3 with hTfR1 and cTfR1, measured as described in Example 9.

FIG. 19 shows SPR sensorgrams of the interaction between the indicated alanine variants of h26D3, studied as scFv building blocks within a bispecific protein format as described in Example 9.

FIG. 20 is an illustration of the two different Gen 2A constructs designed and produced as described in Example 10.

FIG. 21 shows the result of purification of different Gen 2A constructs, produced and purified as described in Example 11. The monomeric content of the bispecific binding proteins was high (in general >98%) and they were produced at low mg/I levels. The purity was analyzed using Coomassie blue SDS-PAGE staining.

FIG. 22 shows sensorgrams from SPR binding analysis of the fourteen Gen 2A binding protein constructs and control, as described in Example 12. One sensorgram for each indicated variant is shown, illustrating that all constructs were functional and bound to hTfR1.

FIG. 23 shows cell binding data for the indicated Gen 2A constructs, measured as described in Example 13. All tested constructs bound in a similar way to cells expressing hTfR1.

FIG. 24 is a collection of diagrams showing the results of CDC measurements on Ramos cells as described in Example 14, with the indicated test constructs #1-7 (“VH first” configuration).

FIG. 25 is a collection of diagrams showing the results of CDC measurements on Ramos cells as described in Example 14, with the indicated test constructs #8-14 (“VL first” configuration).

FIG. 26 is a collection of diagrams showing the results of the in vivo pharmacokinetic study described in Example 15. Plasma and brain exposure is shown after i.v. administration of indicated test constructs, from terminal samples collected at 4, 24, 72, 168 and 240 h, with n=3 mice per timepoint and test construct. Data is presented as mean±SD.

FIG. 27 is a diagram showing plasma concentration versus time profiles from continuous sampling with n=3 mice per test construct after i.v. administration as described in Example 15. Data is presented as mean±SD.

FIG. 28 shows 40× z-stack images of brain cortex of hTfR-KI mice stained for hIgG as described in Example 16, 24 h post-dose. Three replicates (n=3) per indicated group (LC1, HC6 and LC5) are represented. Perfusion score (0-3) of each brain is given within the white squares in lower left corner. Blood vessels are indicated by arrows.

FIG. 29 shows representative 63× z-stack images of each group with hIgG staining vs collagen IV as described in Example 16. Perfusion score (0-3) of each brain is given within the white squares in lower left corner.

FIG. 30 is a pair of diagrams showing the results of the in vivo study described in Example 17. Plasma (A) and brain (B) concentrations at 24 h for the indicated test constructs at the three indicated doses 11 nmol/kg (circles), 40 nmol/kg (triangles) and 60 nmol/kg (inverted triangles). The level of 2A2 #2-8D3 is based on the calibrator prepared from 2A3 #2-WT.

FIG. 31 shows the result of the cytokine response analysis described in Example 17, displaying individual and median plasma levels of the indicated cytokines (A: TNF and KC/GRO; B: IL-6 and IL-10) for the indicated test constructs at the three indicated doses 11 nmol/kg (diamonds), 40 nmol/kg (triangles) and 60 nmol/kg (inverted triangles), at pre-dose and 2 h after dose.

FIG. 32 shows the results of production and purification of a Gen 2D construct as described in Example 18, after gel analysis of purified construct by in-gel protein detection staining with Coomassie blue. The lanes are as follows; (1) Marker; (2) mAb158-2D-h26D3-HC6, non-reduced, (3) mAb158-2D-h26D3-HC6, reduced; (4) mAb158-2D-h26D3, non-reduced; (5) mAb158-2D-h26D3, reduced.

FIG. 33 shows sensorgrams from SPR binding analysis of Gen 2D binding protein constructs and control, as described in Example 19. Sensorgrams for the binding of each indicated variant to both hTfR1 and cTfR1 are shown, illustrating that all constructs are functional and bind to hTfR1 and cTfR1. Fab fragment of h26D3 was included as positive control.

FIG. 34 shows the results of the ADCC assay described in Example 20, using rituximab as positive control. (A) Validation of the assay setup with rituximab which induces a strong fold induction in the presence of target cells (diagram labeled “Rituximab”). Without target cells there is no ADCC induction by rituximab (diagram labeled “Rituximab (w/o Target Cells)”). Similarly tested with and without target cells was the mAb158 antibody, i.e. antibody without attached hTfR1 binding moiety M1. (B) Analysis of the Gen 2D format having h26D3 scFv in the “VL first” configuration as M1 and mAb158 as M2, showing no induction of ADCC. In the same assay, rituximab exhibits a strong induction while the antibody mAb158 alone does not show any indication of ADCC activity.

FIG. 35 shows cell binding data for the indicated Gen 2D construct on K562 cells, measured as described in Example 20.

FIG. 36 shows the results of the in vivo pharmacokinetic study described in Example 21. A: mean (±SD) terminal plasma (filled) and brain (open) concentration-time profiles of mAb000-Gen2D-h26D3-HC6 (diamonds) and mAb000 (squares) in hTfR-KI mice following a single intravenous injection of 40 nmol/kg (n=3-4 per sampling timepoint). B: mean (±SD) plasma concentration-time profiles of mAb000-Gen2D-h26D3-HC6 (diamonds) and mAb000 (squares) from continuous sampling in hTfR-KI mice following a single intravenous injection of 40 nmol/kg (n=4).

FIG. 37 shows frontal cortex 40× Z-stack images of brains from 5×FAD×hTfR-KI mice 72 h post injection with mAb000-Gen2D-h26D3-HC6 (A) or mAb000 (B) as described in Example 22. Total amyloid P and hIgG representative image of two replicates (n=2) per group.

FIG. 38 is a diagram showing competition by indicated test constructs for hTfR1 binding by antibody M-A712 on cell surfaces, measured by flow cytometry as described in Example 23.

FIG. 39 is a diagram showing competition by human ferritin for hTfR1 binding by antibody M-A712 on cell surfaces, measured by flow cytometry as described in Example 23.

### EXAMPLES

While the invention has been described with reference to various exemplary aspects and embodiments, it will be understood by those skilled in the art that various changes may be made and equivalents may be substituted for elements thereof without departing from the scope of the invention. In addition, many modifications may be made to adapt a particular situation or molecule to the teachings of the invention without departing from the essential scope thereof. Therefore, it is intended that the invention not be limited to any particular embodiment, but that the invention will include all embodiments falling within the scope of the appended claims.

The invention will be further illustrated by the following non-limiting Examples. They are offered for illustrative purposes only and are not intended to limit the invention in any manner. Those of skill in the art will readily recognize a variety of non-critical parameters which can be changed or modified to yield essentially the same results. Efforts have been made to ensure accuracy with respect to numbers used (e.g. amounts, temperatures, etc.), but some experimental error and deviations may be present. Unless otherwise indicated, the practice of the invention employs conventional methods of protein chemistry, biochemistry, recombinant DNA techniques and pharmacology, within the skill of the art. Such techniques are explained fully in the existing literature. Additionally, it will be apparent to one of skill in the art that the methods for protein engineering applied herein can also be applied to other constructs described herein and contemplated by the present inventors to fall within the scope of the disclosure.

### Example 1

### Identification of Binders of Human TfR1 by Immunization and Screening

### Immunization and Hybridoma Screening

To identify monoclonal antibodies that bind human transferrin receptor 1 (hTfR1), four 6-10 weeks old Balb/c or C57BL/6 mice were immunized subcutaneously with immunogen together with adjuvant. The hTfR1 immunogen was designed to contain the ectodomain of the human TfR1 protein, N-terminally fused to a T-cell epitope from tetanus toxin, P2 (Kovacs-Nolan and Mine (2006), Biochim Biophys Acta 1760:1884-1893) via a GSS linker, and an N-terminal 10× histidine tag (His₁₀-P2-hTfR1; SEQ ID NO:71). Following gene construction, recombinant His₁₀-P2-hTfR1 protein was generated by transient transfection in Hek293 cells using the Expi293™ Expression system (Gibco), purified on a nickel column (HisTrap FF, cat. no. 17-5255-01, GE Healthcare), buffer exchanged to PBS and concentrated to 1 mg/ml. Expressed TfR1 immunogen was aliquoted and stored at −80° C. until use. Quil-A adjuvant (vac-quil, InvivoGen) was used for all immunizations except for the final booster injection in which no adjuvant was included. For use, Quil-A was resuspended in ddH₂O at a concentration of 1 mg/ml, sterile filtered and aliquoted in 0.1 ml aliquots stored at −80° C. Quil-A was administered at a dose of 10 ag/mouse.

Animals were immunized every month with the recombinantly produced immunogen, His₁₀-P2-hTfR1, mixed and co-administered with Quil-A. Three weeks after each immunization, blood samples were collected, and the plasma was analyzed for presence of antibodies reactive towards recombinantly produced human TfR1 and mouse TfR1. Titers were considered high enough when the ELISA response at 1/100,000 dilution exceeded the average of the blanks (i.e. background) plus 3 standard deviations of the blanks. The four mice used in this study received between 4 and 6 immunizations each.

Three days before fusion, the final intraperitoneal booster injection was given to the mice in absence of adjuvant. At sacrifice, mice were anesthetized with isoflurane. Intact spleens were collected by opening the abdominal cavity and dissected. Briefly, a single cell suspension of the spleen from an immunized mouse was prepared and mixed with Sp2/0 cells at a 3:1 ratio. The cells were fused using PEG and the cells were added to a bottle of ClonaCell™-HY Medium D (STEMCELL Technologies). 60-70 μl per well was then dispensed into 96-well plates. After 6-7 days, 150 μl HAT-medium was added to each well in the semi solid 96-well plates. The day after, 120 μl of supernatant was discarded from each well and 100 μl fresh HAT-medium was added. The next day, 100 μl of the supernatant of each well was taken and transferred to a storage plate and tested for presence of antibodies against mouse TfR1 using indirect ELISA on nickel-coated plates according to the protocol below. A repeated screen of the hybridoma plates was performed by adding 120 μl HAT-medium on day 12 and by 3 days later transferring 25 μl supernatant to ELISA plates to screen for reactivity against mouse TfR1 (both screens referred to as “primary screen”). Clones that were positive towards mouse TfR1 with OD>0.2 were transferred to 24-well plates, cultured for at least 3 days, and subjected to a secondary screen for reactivity towards murine, human and cynomolgus TfR1 in solution using biolayer interferometry (BLI) (referred to as “secondary screen”). Whereas binding of both hTfR1 and cynomolgus TfR1 was indicated, only very weak or no binding was detected for mTfR1 in the secondary screen. Supernatants from 24-well plates were also screened for binding towards His-tagged hTfR1 as well as lack of binding towards His-tagged amyloid-β precursor protein (APP; negative control) using both direct coated TfR1 plates and nickel-coated plates as described below. Binding towards cynomolgus TfR1 (cTfR1) was also analyzed using direct TfR1 coat. Notably, ELISA responses (OD450 values) were very low for mTfR1 compared to hTfR1 and cTfR1, indicating weaker binding to mTfR1 compared to the binding to hTfR1 and cTfR1 for all positive clones.

Selected clones were diluted using limiting dilution assays (LDA) to reach monoclonality. Reactivity against mouse TfR1 and human TfR1 were re-tested by ELISA on monoclonal cultures following LDA and expansion.

### Indirect ELISA Screening

ELISA assays were performed according to standard ELISA protocols in order to screen plasma samples for reactivity towards the target antigens after immunizations, or to identify hybridoma clones producing antibodies with reactivity against the TfR1 target protein. Briefly, 96-well half area plates (Corning) were coated with 1 μg/ml His₁₀-mTfR1 (SEQ ID NO:72) or His₁₀-hTfR1 (SEQ ID NO:73). His₁₀-mTfR1 and His₁₀-hTfR1 were recombinantly produced and purified using the procedure described above for the His₁₀-P2-hTfR1 immunogen. The plates were blocked with 150 μl/well of protein free blocking solution (Pierce) for 1 h at room temperature with shaking (600-900 rpm). The plates were washed four times with PBS containing 0.1% TWEEN®-20 and Kathon™. Plasma samples serially diluted from a starting dilution of 1/450 or hybridoma supernatants diluted ½ were added to the plates (50 μl/well; dilution buffer: PBS with 0.1% BSA and 0.05% TWEEN®-20) and incubated for 2 h at room temperature and then the plates were washed four times. Detection antibody (HRP-conjugated anti-mouse IgG, Southern Biotech, cat. no. 1030-05, diluted 1/5000 in dilution buffer) was added at 50 μl/well, and the plates were incubated for 1 h at room temperature. After another wash (as above), 50 μl/well TMB substrate (K-Blue® Aqueous, Neogen) was added, and the reaction was stopped after 10-15 min with 50 μl/well of 0.5 M H₂SO₄. The optical density at 450 nm was read using a plate reader (Tecan). The endpoint titers were defined as the dilution above the average of the blank wells (background) plus 3 standard deviations of the blank wells.

The primary screen of hybridoma clones producing antibodies with reactivity against the target protein was performed using nickel-coated ELISA plates. Briefly, 96-well Ni-coated plates (PIERCE) supplied pre-blocked with BSA were incubated with 3 μg/ml (100 μl) His₁₀-mTfR1 without shake overnight at 4° C. The plates were washed four times with PBS containing 0.1% TWEEN®-20 and Kathon™. Hybridoma supernatants diluted ¼ were added to the plates (dilution buffer: PBS with 0.1% BSA and 0.05% TWEEN®-20) and incubated for 2 h at room temperature and then the plates were washed four times. Detection antibody (HRP-conjugated anti-mouse IgG, Southern Biotech, cat. no. 1030-05, diluted 1/5000 in dilution buffer) was added at 100 μl/well, and the plates were incubated for 1 h at room temperature. After another wash (as above), 100 μl/well of K-Blue® Aqueous substrate (Neogen) was added, and the reaction was stopped after 10-15 min with 100 μl/well of 0.5 M H₂SO₄. The optical density at 450 nm was read using an ELISA plate reader (Tecan).

Examples of clones considered to be positive in binding mouse TfR1 and human TfR1 are shown in Table 1. These clones were also confirmed to bind both His-tagged hTfR and cTfR by ELISA, and to lack binding to His-tagged APP (negative control). Selected clones were further characterized in various assays.

TABLE 1
Examples of identified clones from hybridoma screening

| | mTfR1 | | | | |
|---|---|---|---|---|---|
| | specificity by | hTfR | cynoTfR | mTfR1 | Isotype |
| | indirect ELISA | binding | binding | binding | (from |
| Clone | (Ni capture) | by BLI | by BLI | by BLI | sequencing) |
| 24B4 | OD > 0.2 | Yes | Yes | No/weak | IgG2a/κ |
| 26D3 | OD > 0.2 | Yes | Yes | No/weak | IgG2a/κ |
| 37D10 | OD > 0.2 | Yes | Yes | No/weak | IgG2a/κ |

### Biolayer Interferometry Measurements

Selected clones were investigated using biolayer interferometry (BLI) on an Octet instrument (Octet Red384, ForteBio). In the setup used, the adopted method involves capture of IgG from the respective clone on the individual sensor tips to allow for detection of antibodies that bind to target in solution. In addition to providing a measure of binding, BLI measurements provide more details about the overall binding properties, because they include estimates of the on-rate and off-rate.

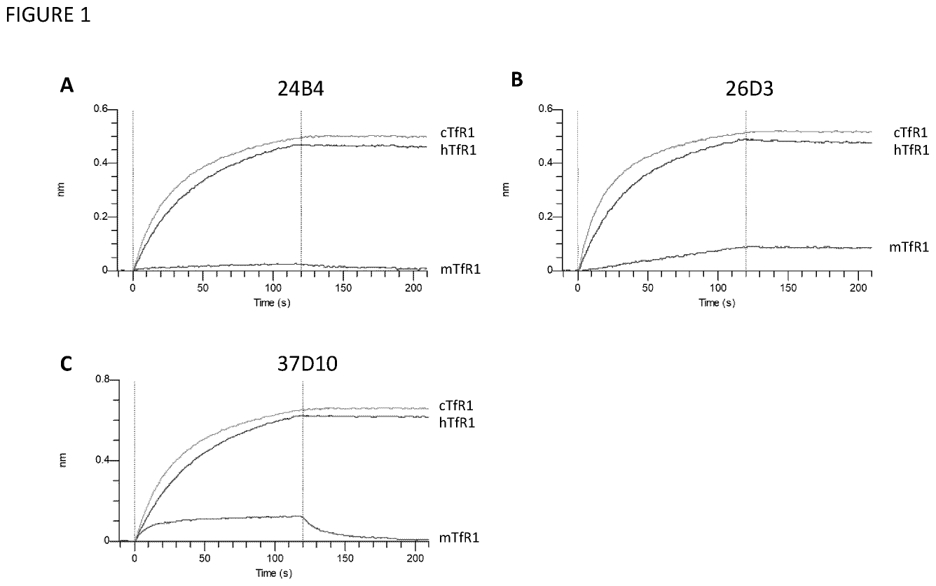

FIG. 1 shows the results of BLI measurements for three selected clones provided as examples, with binding measured directly in the crude hybridoma supernatant. Briefly, mouse IgG antibody clones in hybridoma supernatants, diluted 1:1 in running buffer (PBS, 0.02% TWEEN®-20 and 0.01% BSA), were captured on anti-mouse capture biosensors (anti-mouse capture, AMC, Molecular devices, Cat. 18-5580). Next, sensors with immobilized IgGs were briefly washed for 10 s before incubation in running buffer to establish a baseline signal. Association to target antigens were measured by incubating sensors for 120 s in wells of the assay plate containing the following concentrations of respective target antigen: 500 nM mTfR1, 250 nM hTfR1 and 250 nM cTfR1. All proteins were diluted in running buffer. Target dissociation was measured by incubating the biosensors in running buffer for 90 s. All tested clones, i.e. 24B4, 26D3 and 37D10, bind to both human and cynomolgus TfR1 but very weakly to mouse TfR1. Overall, most clones showed more cross-reactivity towards human and cynomolgus TfR1 than against mouse TfR1.

### Sequencing of Selected Clones

Clones of interest were cryopreserved and sequenced by whole transcriptome shotgun sequencing. Among the sequenced hybridoma clones were clones denoted 26D3, 24B4 and 37D10. The amino acid sequences obtained for the respective heavy chain variable (VH) and light chain variable (VL) regions of these antibodies are given in Table 2 below:

TABLE 2
Variable region amino acid sequences
for selected primary antibodies

| | | SEQ |
|---|---|---|
| | Antibody | ID |
| Region | Amino acid sequence | NO: |
| | 26D3 | |
| VH | EVQLQQFGVELVKPGASVKISCKASGYIFTD | 65 |
| | YNMDWVKQSHGKSLEWIGDINPDYDTTSYNE | |
| | KFKGKATLTVDKSSSTAYMELRSLTSEDTAV | |
| | YYCARGGYSGSSYYHPMDYWGQGTSVTVSS | |
| VL | DIVMSQSPSSLAVSVGEKITMSCKSSQSLLY | 66 |
| | STNQKNYLAWYQQKPGQSPELLIYWASTRES | |
| | GVPDRFTGSGSGTDFTLTISNVRAEDLAVYY | |
| | CQQYFIYPRTFGGGTKLEIK | |
| | 24B4 | |
| VH | EVQLQQFGAELVKPGTSVKISCKASGYTFTD | 67 |
| | YNMDWVKQGHGKGLEWIGDINPNYDTTSYSQ | |
| | KFKGKATLTVDKSSSTAYMELRSLTSEDTAV | |
| | YYCARSEAGNYYWYFDVWGAGTTVTVSS | |
| VL | DIVMSQSPSSLAVSVGEKVTMSCKSSQSLLY | 68 |
| | SSNRKNYLAWYRQKPGQSPKLLIYWASTRES | |
| | GVPDRFTGSGSGTDFTLTISSVKAEDLAVYS | |
| | CQQYYNYPYTFGGGTKLEIK | |
| | 37D10 | |
| VH | QVQLQQSGAELVRPGTSVKISCKASGYAFTN | 69 |
| | YWLGWVKQRPGHGLEWIGDIFPGSDNTYYNE | |
| | KFKGKATLTADKSSSTAYMQLSSLASEDSAV | |
| | YFCARSGNFYAMDYWGQGTSVTVSS | |
| VL | ENVLTQSPAIMSASPGEKVTMTCSASSSVNY | 70 |
| | MNWYQQKSSTFPKLWIYDTSKLASGVPGRFS | |
| | GSGSGKFYSLTISSMEAEDVATYYCFQGSGY | |
| | PFTFGSGTKLEIK | |

The complementarity determining regions (CDRs) of these antibodies were identified using the Kabat definition, and are given in Table 3 below.

TABLE 3
CDR sequences of primary antibodies

| Antibody | | | |
|---|---|---|---|
| | VHCDR1 | VHCDR2 | VHCDR3 |
| 26D3 | DYNMD | DINPDYDTTSYNEKFKG | GGYSGSSYYHPMDY |
| | (SEQ ID NO: 10) | (SEQ ID NO: 11) | (SEQ ID NO: 12) |
| 24B4 | DYNMD | DINPNYDTTSYSQKFKG | SEAGNYYWYFDV |
| | (SEQ ID NO: 10) | (SEQ ID NO: 34) | (SEQ ID NO: 35) |
| 37D10 | NYWLG | DIFPGSDNTYYNEKFKG | SGNFYAMDY |
| | (SEQ ID NO: 38) | (SEQ ID NO: 39) | (SEQ ID NO: 40) |
| | VLCDR1 | VLCDR2 | VLCDR3 |
| 26D3 | KSSQSLLYSTNQKNYLA | WASTRES | QQYFIYPRT |
| | (SEQ ID NO: 13) | (SEQ ID NO: 14) | (SEQ ID NO: 15) |
| 24B4 | KSSQSLLYSSNRKNYLA | WASTRES | QQYYNYPYT |
| | (SEQ ID NO: 36) | (SEQ ID NO: 14) | (SEQ ID NO: 37) |
| 37D10 | SASSSVNYMN | DTSKLAS | FQGSGYPFT |
| | (SEQ ID NO: 41) | (SEQ ID NO: 42) | (SEQ ID NO: 43) |

### Example 2

### In Vitro Binding to Human and Cynomolgus TfR1 and Epitope Screen

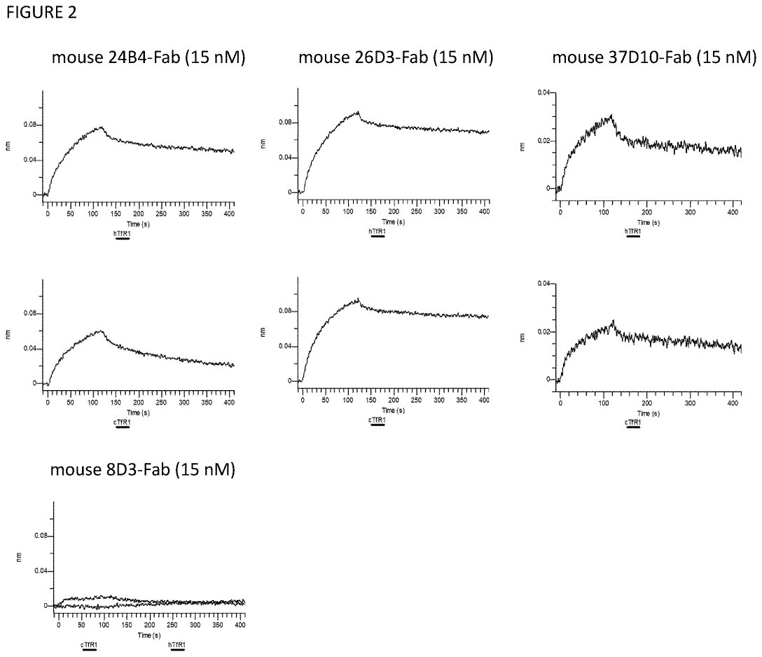

A more detailed binding analysis by BLI was performed on purified, selected antibodies. Binding of Fab fragments from the murine antibodies 26D3, 24B4 and 37D10 to human TfR1 and cynomolgus TfR1 was investigated. For example, the BLI instrument Octet Red384 was used to measure binding between immobilized TfR1 and the tested Fab fragments in solution. Antibody binding to TfR1 was measured with TfR1 complexed to the human transferrin ligand (Tf). Tf/TfR1-complexes were formed on streptavidin biosensors by first loading sensors with biotinylated human holo-transferrin followed by a complex-formation step by capturing either hTfR1 or cTfR1 on the sensors. Final complex density on the sensors was similar for both hTfR1 and cTfR1. Antibody binding to TfR1 was measured during an association phase of 120 s and a dissociation phase of 300 s. FIG. 2 shows sensorgrams for 15 nM of each of 24B4-Fab, 26D3-Fab and 37D10-Fab, as well as for a Fab derived from the known TfR1 binding antibody 8D3 (Boado et al (2009), Biotechnol Bioeng 102:1251-1258). The data indicate a similar binding profile against human and cTfR1 for both 24B4-Fab and 26D3-Fab, and cross-reactive binding to both species is also detected for 37D10-Fab, while no significant binding of 8D3-Fab against human or cynomolgus TfR1 was detected. Importantly, the experiment shows that 24B4-Fab, 26D3-Fab and 37D10-Fab all bind to TfR1 when the natural ligand transferrin is in complex with TfR1.

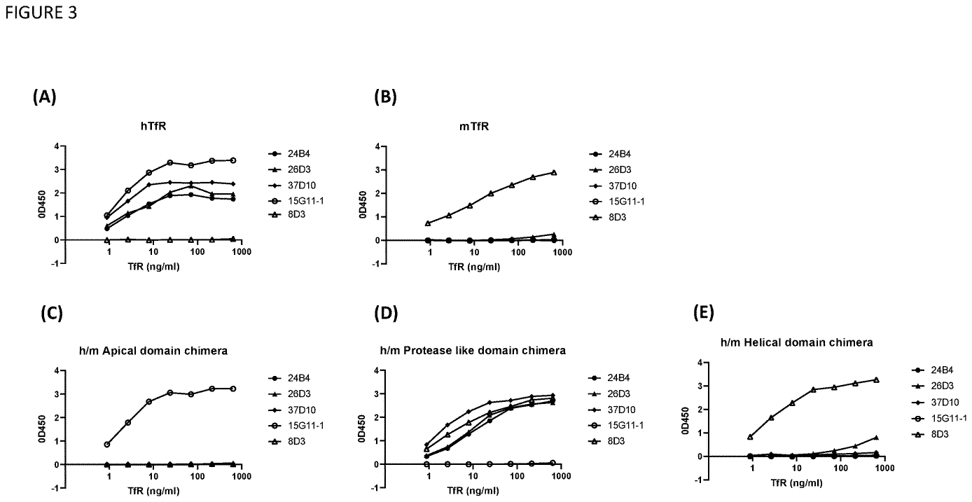

Next, an ELISA experiment showed that antibodies 26D3, 24B4 and 37D10 bind to the protease-like domain of TfR1. In the ELISA experiment, human, mouse or three different chimeric TfR1 receptors were used to coat ELISA plates (FIG. 3). The ELISA protocol was slightly modified as follows from the indirect ELISA described in Example 1. Briefly, ELISA plates were coated with the following His-tagged antigens at 1 μg/ml: ectodomain of human TfR1 (His10-hTfR1; SEQ ID NO:74), ectodomain of mouse TfR1 (His10-mTfR1; SEQ ID NO:75), chimeric TfR1 consisting of human apical domain grafted on mouse TfR1 ectodomain (h/m apical domain chimera, mhHD_TFR1; SEQ ID NO:76), chimeric TfR1 consisting of human helical domain grafted on mouse TfR1 ectodomain (h/m helical domain chimera, mhHD_TfR1; SEQ ID NO:77) or chimeric TfR1 consisting of human protease like domain grafted on mouse TfR1 ectodomain (h/m protease-like domain chimera, mhPLD_TfR1; SEQ ID NO:78). The coated plates were then blocked. Dilution series of mouse IgG of the analyzed antibodies were prepared in PBS and incubated on the ELISA plates. Unbound antibodies were then washed off before incubating wells with a HRP-conjugated secondary, anti-mouse-IgG for 1 h. Plates were then washed again before addition of HRP substrate TMB for development and detection of antibody binding to the wells. TMB development was stopped by adding 0.5 M H₂SO₄ to the wells and ELISA responses measured as the OD at 450 nm in an ELISA plate reader. As illustrated in FIG. 3, 26D3, 24B4 and 37D10 only bind hTfR1 (A) and not mTfR1 (B). There is no binding of 26D3, 24B4 or 37D10 to the construct with the human apical domain grafted onto the rest of the mTfR1 ectodomain (C). The control antibody 15G11-1 (Yu et al (2014), Sci Transl Med 6:261ra154) known to bind to the human apical domain shows binding to the h/m apical domain chimera as expected (C). In addition, 26D3, 24B4 and 37D10 bind to the h/m protease-like domain chimera (D), but not to any of the plates coated with the other chimeric receptors (C and E). Further, the control antibody 8D3, with an epitope in the apical domain of mTfR1, binds to all plates coated with TfR1 antigens including this domain, namely mTfR1 (B), h/m protease-like domain chimera (D) and h/m helical domain chimera (E), In summary, the experiment demonstrates that the epitope or epitopes for 26D3, 24B4 and 37D10 lie(s) predominantly within the protease-like domain of hTfR1, and that this is in contrast to the control antibodies 15G11-1 and 8D3.

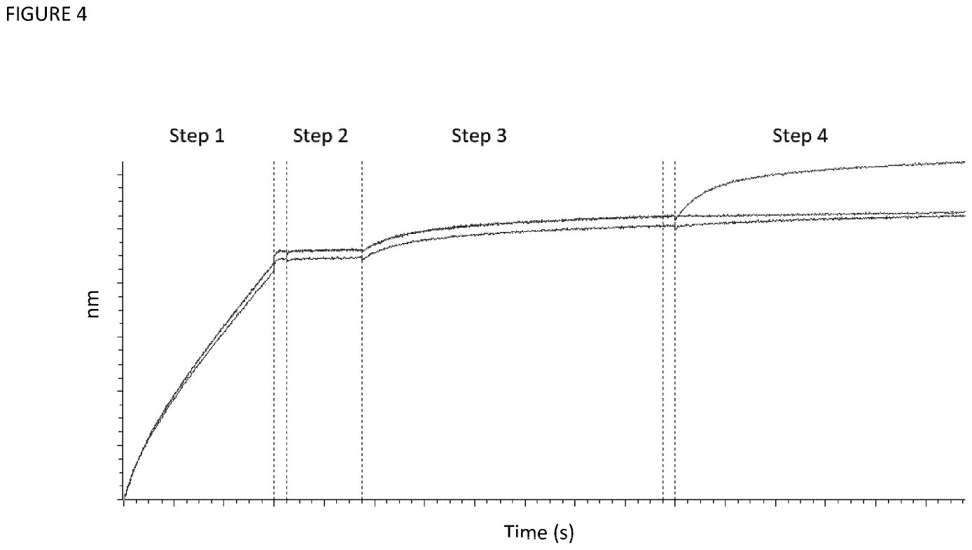

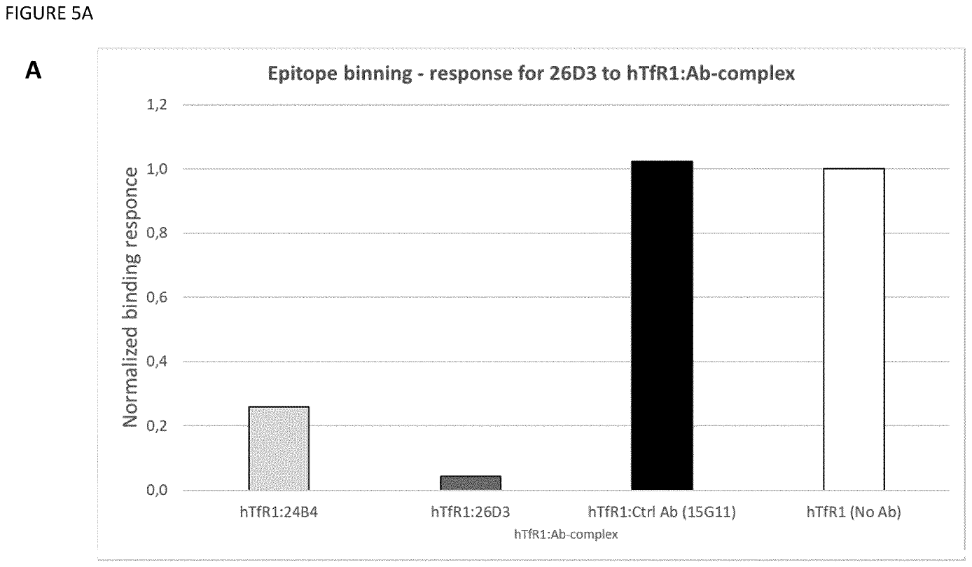

In a further BLI experiment carried out for the purpose of epitope binning (binding competition), it was then shown that binding by both 26D3 and 24B4 is targeted to the same or overlapping regions of hTfR1, with an epitope located outside the apical domain (FIG. 4). The epitope binning experiment by BLI was conducted on an Octet Red384 instrument (ForteBio) by first (Step 1) immobilizing biotinylated hTfR1 to streptavidin biosensors (High precision biosensors, ForteBio). Next (Step 2), a washing step was carried out. Then (Step 3), hTfR1 loaded sensors were incubated in either buffer (non-competitive reference) or 200 nM of the respective antibody (Ab) to form hTfR1:Ab complexes on the sensors. Finally (Step 4), sensors with free hTfR1 (reference) or respective preformed hTfR1:Ab complex was incubated in 200 nM of respective antibody to measure binding to hTfR1 in complex with the competing antibody. FIG. 4 shows representative BLI sensorgrams obtained during the indicated main assay steps. The signal in Step 4 is indicative of the degree of competition between the two analyzed antibodies. If the antibodies compete for binding to the same or overlapping epitope, there is no increase in the signal of the sensorgram in Step 4. Conversely, if the two tested antibodies bind to distinct and different epitopes, there will be an increased signal from Step 4.

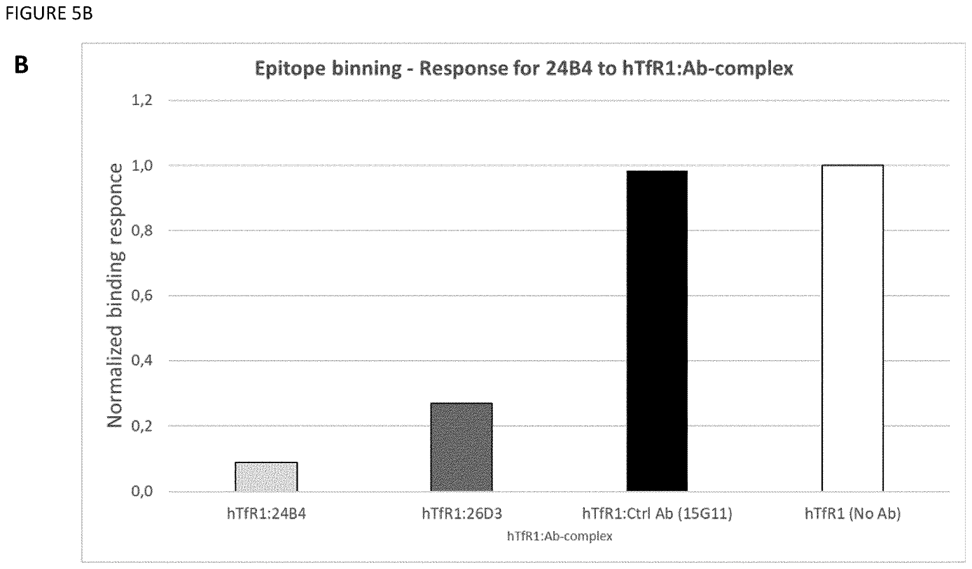

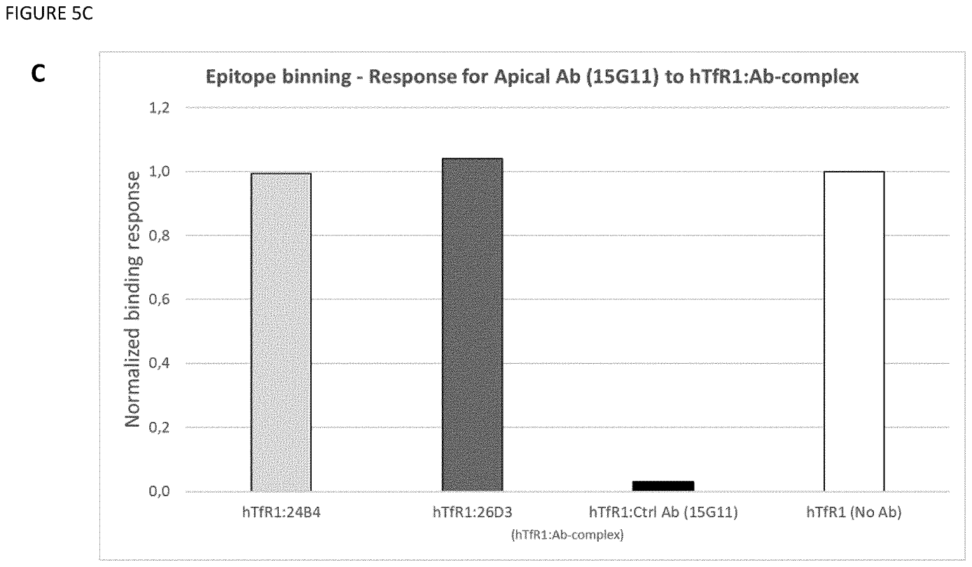

The results of competitive screening of antibody binding to epitopes on hTfR1 by epitope binning as described above is illustrated in FIG. 5. Antibodies 26D3 (dark grey bars) and 24B4 (light grey bars) were shown to bind to an overlapping epitope, which is distinct from the hTfR1 apical domain epitope of control antibody 15G11-1 (black bars). FIG. 5A shows that the binding response for 26D3 is reduced by over 70% when hTfR1 is in complex with 24B4. As expected, binding of 26D3 to pre-formed hTfR1:26D3-complex is nearly fully inhibited, illustrating that it blocks itself. Similarly, FIG. 5B shows that the binding response for 24B4 is 70% lower when hTfR1 is in complex with 26D3 and nearly fully inhibited by itself. Both 24B4 and 26D3 retain the full binding response to hTfR1 when hTfR1 is in complex with the control antibody 15G11-1, which has its binding epitope within the apical domain of hTfR1 (FIGS. 5A and 5B, black bars). As shown in FIG. 5C, the control antibody 15G11-1 has similar binding responses to the apical domain of hTfR1, regardless of whether it is tested against hTfR1 without competition antibody or when the receptor is in complex with 24B4 or 26D3. In FIG. 5, all responses were normalized to the respective antibody's maximal binding response to free hTfR1.

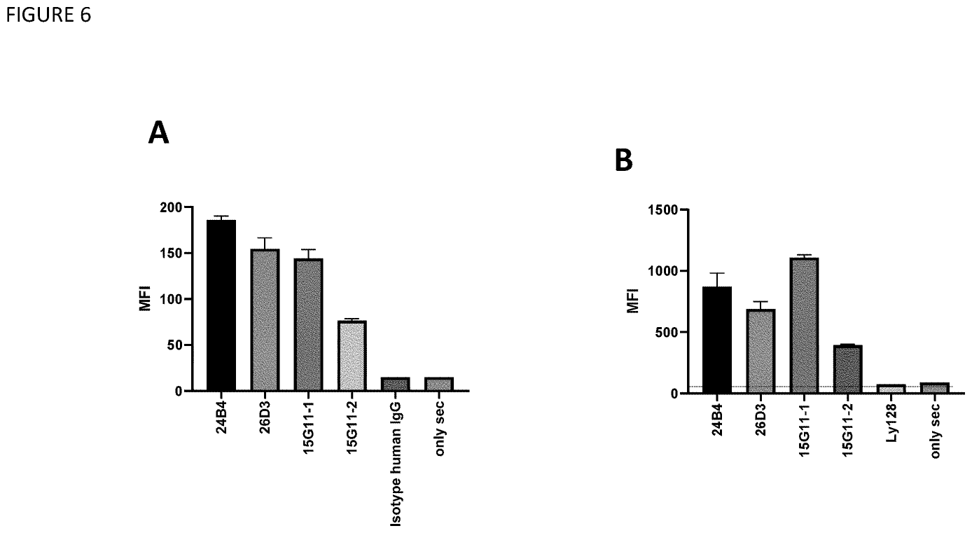

Furthermore, antibody binding to endogenous hTfR1 on brain endothelial cells was studied. Binding to endogenous hTfR1 on cell surfaces was monitored using flow cytometry and human hCMEC/D3 cells (Weksler et al (2013), Fluids Barriers CNS 10:16), which are known to express significant levels of hTfR1 on their surface. Cells that stained positively were plotted and the mean fluorescence intensity (MFI) is shown in FIG. 6. Both FIG. 6A (IgG1 antibodies) and 6B (Fab fragments) show that cells were positively stained for hTfR1 with 24B4 and 26D3 to a similar degree (MFI) compared to the positive control antibody 15G11-1 having a high hTfR1 affinity and to a higher degree than the low affinity control antibody 15G11-2 (Yu et al (2014), supra). No background staining was detected with the negative isotype control (FIG. 6A) or the non-related Fab fragment Ly128 (FIG. 6B). These data illustrate that both 24B4 and 26D3 bind to hTfR1 expressed on a cell surface.

### Example 3

### Competition for hTfR1 Binding with Ferritin and Transferrin

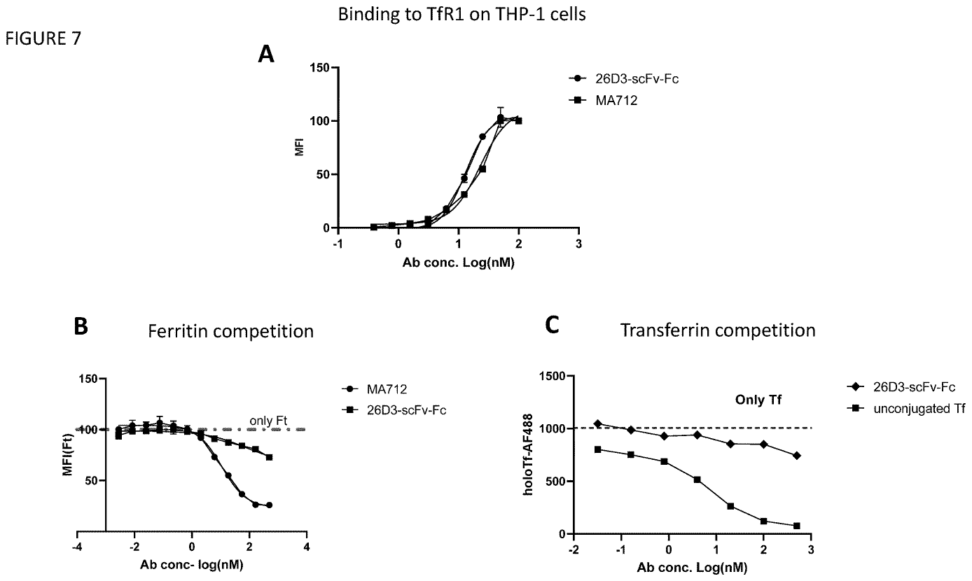

The unique binding to hTfR1 of the binders according to the disclosure, binding to the protease-like domain of hTfR1 and identified as described in Example 1, was evaluated for competition with natural TfR1 ligands ferritin (Ft) and transferrin (Tf). In order to test ferritin competition with antibody, the human monocytic cell line THP-1 (Sigma/ECACC) was used. Binding of the scFv-Fc format (see Example 4 below) and control antibody (M-A712) to hTfR1 on the THP-1 cell surface was confirmed, as shown in FIG. 7A. For evaluating the competition between ferritin and the disclosed binders, cells were incubated with serially diluted test binders along with ferritin from human liver (BioRad, 4420-4804) for 1 h at 4° C. After incubation, ferritin that had bound to hTfR1 on the cell surface was captured using a primary sheep antibody against human liver ferritin (BioRad, AHP2179G) and analyzed using flow cytometry. The results are displayed in FIG. 7B, and show that the 26D3 scFv-Fc does not compete with ferritin on the cell surface, whereas the control antibody anti-CD71, clone M-A712, known to bind to the same epitope on hTfR1 as Ft (Maier et al (2016), Mol Ther Nucleic Acids 5:e321) clearly competes with Ft binding. Also for the identified 26D3 hTfR1 binder, the impact on Ft binding is much less, illustrating that 26D3 has a different epitope on hTfR1 than the binding site for Ft (FIG. 7B).

For transferrin competition, K562 lymphoblast cells (Sigma/ECACC) were used. Cells were incubated with serially diluted test constructs along with Alexa Fluor 488 conjugated, human holo-transferrin (Thermo Fisher; T13342) and incubated for 1 h at 4° C. Transferrin bound to hTfR1 on cell surfaces was captured using flow cytometry, and the mean fluorescence intensity was plotted. FIG. 7C shows that there is no competition between the 26D3 binder and transferrin. When non-labeled (unconjugated) Tf was used as positive control for competition, the binding of labeled (AF488) Tf signal was reduced in a concentration dependent way. The experiment illustrates that a binder directed against the protease-like domain of TfR1 does not compete directly for the same epitope as transferrin.

Overall, this example shows that binding of 26D3 to hTfR1 does not negatively affect the ability of the two endogenous ligands ferritin and transferrin to bind to the receptor.

### Example 4

### Humanization of hTfR1 Binder 26D3

The Fab sequence of mouse antibody 26D3, identified and characterized as described in Examples 1-3, was analyzed and an in silico model of the 26D3 Fab 3D structure was generated using Bioluminate Software (Schrödinger). This murine Fab model was used as input for humanization. In this process, the CDRs of the VH and VL regions of 26D3 (see Table 3; SEQ ID NO:10-15) were grafted in silico into various human variable domains and some residues were back mutated to murine framework at some positions. Three variants having the fewest back mutations and otherwise desirable characteristics were generated and extracted from the software. One such humanized variant was selected for expression and denoted h26D3. h26D3 has the VH region sequence defined in SEQ ID NO:44 and the VL region sequence defined in SEQ ID NO:58. The humanized version h26D3 and the murine original sequence 26D3 were both expressed as His-tagged Fabs by transient transfection of Chinese Hamster Ovary cells (ExpiCHO; Thermo Fisher Scientific) according to the manufacturer's instructions. The harvested supernatant was purified using HiTrap IMAC Sepharose FF (Cytiva) followed by a size exclusion chromatography on HiLoad Superdex 200 pg 26/600 (Cytiva). The following buffers were used: Ni-NTA wash buffer: 20 mM Tris pH 8.0, 10 mM imidazole and 200 mM NaCl; Ni-NTA elution buffer: 20 mM Tris pH 8.0, 200 mM NaCl and 500 mM imidazole; size-exclusion buffer (SEC): 1×dPBS (Thermo Fisher).

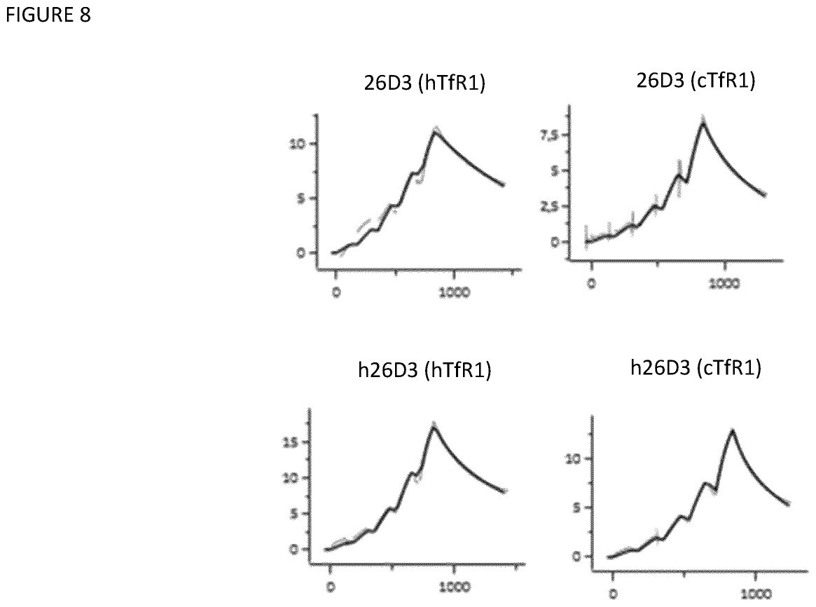

Binding of the purified Fabs to human and cynomolgus TfR1 was evaluated using surface plasmon resonance (SPR) on a Biacore 8K instrument (Cytiva) and the results are shown in FIG. 8. 1 μg/ml of human TfR1 (truncated hTfR1 of SEQ ID NO:86) or cynomolgus TfR1 (truncated cTfR1 of SEQ ID NO:87) was immobilized on a Cm5 sensor chip (Cytiva, #BR100399) using the amine coupling kit type 2 (Cytiva, #BR100633) according to the manufacturer's instruction. The h26D3 and 26D3 Fabs were injected over the chip using a 2-fold dilution series in five steps starting at 25 nM. The interaction was measured using the single cycle kinetics method with a contact time of 120 s at a flow rate of 30 μl/ml followed by a dissociation time of 600 s. Regeneration of the surface between cycles was done by injecting 3M MgCl₂. The binding data were fitted to a 1:1 interaction model. The Fabs were diluted in HBS-EP+ (Cytiva, #BR100669). Experiments were performed at 25° C. The data confirm that the humanized variant of 26D3, i.e. h26D3, retained binding capacity for human and cynomolgus TfR1 (FIG. 8). The kinetic parameters obtained in the experiment are given in Table 4 below.

TABLE 4
SPR analysis of murine and humanized
26D3 Fabs vs. hTfR1 and cTfR1

| Tested binder | Target | kₐ (1/Ms) | k_{d} (1/s) | K_{D} (M) |
|---|---|---|---|---|
| 26D3 | hTfr1 | 2.8 × 10⁶ | 1.0 × 10⁻³ | 3.6 × 10⁻⁹ |
| 26D3 | cTfR1 | 5.7 × 10⁶ | 6.2 × 10⁻³ | 1.0 × 10⁻⁸ |
| h26D3 | hTfr1 | 1.2 × 10⁶ | 4.5 × 10⁻³ | 3.2 × 10⁻⁹ |
| h26D3 | CTfR1 | 2.7 × 10⁶ | 1.5 × 10⁻³ | 5.8 × 10⁻⁹ |

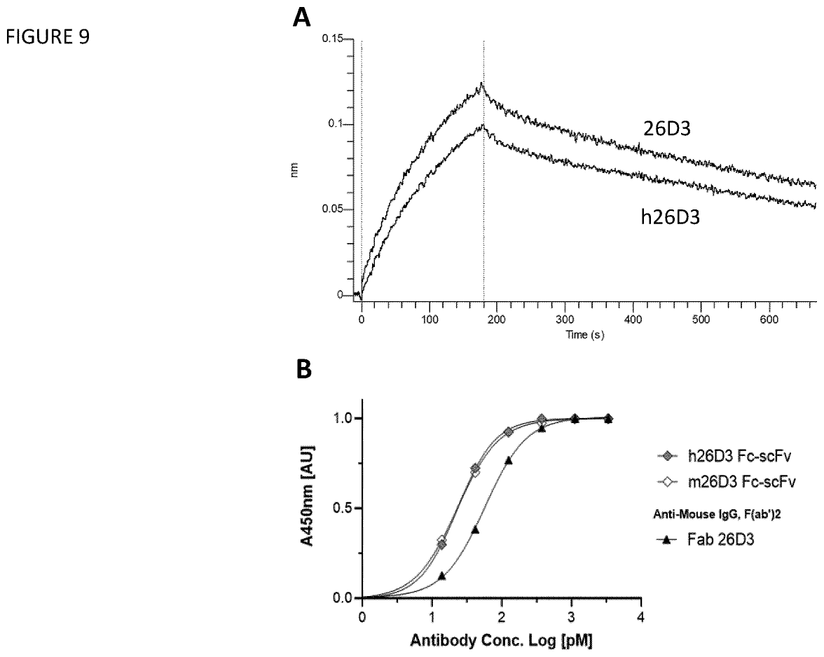

Both murine 26D3 and the humanized variant h26D3 were converted to the scFv format and confirmed to have maintained target binding as scFv (FIG. 9). Murine and humanized 26D3 were reformatted to scFv (SEQ ID NO:79 and SEQ ID NO:80 respectively) and produced as monovalent Fc-fused scFv antibody fragments by employing the knob-into-hole (KiH) technology. In this format, one scFv fragment is fused only to the knob half of the Fc (SEQ ID NO:81), while the hole half of Fc (SEQ ID NO:82) is left unfused. The resulting antibody format is a one-armed scFv-Fc. The 26D3 scFv fused to the knob half of the Fc has the complete amino acid sequence SEQ ID NO:83, whereas the h26D3 scFv fused to the knob half of the Fc has the complete amino acid sequence SEQ ID NO:84. The binding profiles for murine and humanized 26D3 in this scFv format are similar and confirm binding activity in the scFv format. Binding responses agree with those of the antibody in Fab format. This was confirmed by several methods, including a kinetic experiment using BLI (results shown in FIG. 9A) and an ELISA (results shown in FIG. 9B). Binding kinetics for murine and humanized 26D3-scFv-Fc were measured by BLI by first immobilizing biotinylated hTfR1 to streptavidin biosensors (Fortebio). Sensors were then washed in buffer (Kinetics buffer, Fortebio) before measuring association of 26D3-scFv-Fc (murine) and h26D3-scFv-Fc (humanized) at 25 nM concentrations followed by a 500 s dissociation phase. In the ELISA experiment, hTfR1 was used to coat the plates for standard binding ELISA experiments using the protocol for indirect ELISA described in Example 1.

### Example 5

### Crystallization and Structure Determination of h26D3-Fab in Complex with hTfR1

This example describes crystallization of a complex between h26D3-Fab and hTfR1 and determination of the binding interface. Ectodomain of human TfR1 (SEQ ID NO:74) was expressed by transient transfection of human embryonic kidney cells (Expi297; Thermo Fisher Scientific) according to the manufacturer's instructions. The harvested supernatant was purified using HiTrap IMAC Sepharose FF (Cytiva) followed by size exclusion chromatography on HiLoad Superdex 200 pg 26/600 (Cytiva). The buffers used and purification of the humanized Fab were as described in Example 4.

The formation of a complex between humanized h26D3-Fab and hTfR1 was done by mixing of the two components at a molar ratio of 1:1 in 1×dPBS and incubation at room temperature for 1 h. Subsequently, the complex was purified using size exclusion chromatography on HiLoad Superdex 200 pg 26/600 (Cytiva) as described in Example 4.

Crystallization was performed using a stock solution of hTfR1-h26D3 at 15 mg/ml in PBS which was diluted to 4 mg/ml in PBS supplemented with 4 mM β-mercaptoethanol. A 100+100 nl drop was set up using the additive screen in reservoir: 0.1 M sodium potassium phosphate pH 6.5, 10% PEG 3000, 0.05% dichloromethane and 2 mM β-mercaptoethanol. The crystal was flash-frozen in reservoir solution supplemented by 8% glycerol and 16% PEG 400.

X-ray data collection and refinement were performed as follows. Data was collected to 3.87 Å at Diamond Light Source beamline 104. The beamline was equipped with a DECTRIS Eiger2 XE 16M detector. The data set was integrated using XDS (Kabsch (2010), Acta Crystallogr D Biol Crystallogr 66:125-132) with STARANISO anisotropic scaling (Tickle et al (2018), Global Phasing Ltd) and diffracted to 3.87 Å along the c* direction of the reciprocal lattice, and to 4.82 Å in the a*/b* plane. Three complexes were found in the asymmetric unit. The structure was refined using the Buster refinement software and model building was carried out in Coot. Data collection and refinement parameters and statistics are given in Table 5 below.

TABLE 5
X-ray diffraction data collection and refinement statistics

| | |
|---|---|
| Resolution (Å) | 110.81-3.87 (4.27-3.87) |
| Wavelength (Å) | 0.97950 |
| Space group | P31 2 1 |
| Unit cell (Å) | a = b = 127.95, c = 472.91 |
| Spherical completeness (%) | 63.9 (12.7) |
| Ellipsoidal completeness (%) | 93.8 (74.0) |
| No. of observations/unique reflections | 546922/27520 (27874/1378) |
| Redundancy | 19.9 (20.2) |
| <I/σ(I)> | 9.8 (1.7) |
| CC(1/2) (%) | 99.9 (81.5) |
| Rmerge (I) (%) | 20.6 (200.9) |
| Rpim (I) (%) | 6.6 (62.8) |
| Rmodel (F) (%) | 26.0 (31.6) |
| Rfree (F) (%) | 31.4 (34.3) |
| No. of non-hydrogen atoms | 24858 |
| No. of water molecules | 0 |
| rms deviations from ideal geometry | |
| Bond lengths (Å) | 0.009 |
| Bond angles (°) | 1.1 |
| Mean B-factor protein chain | 76.9/110.0/103.0/132.2/ |
| A/B/C/D/E/F/G/H/L (Å²) | 134.4/87.2/101.0/79.9/83.4 |
| Mean B-factor glycosylations (Å²) | 70.7 |
| Ramachandran plot quality | |
| Favored regions (%) | 93.4 |
| Allowed regions (%) | 4.3 |
| Outliers (%) | 2.3 |

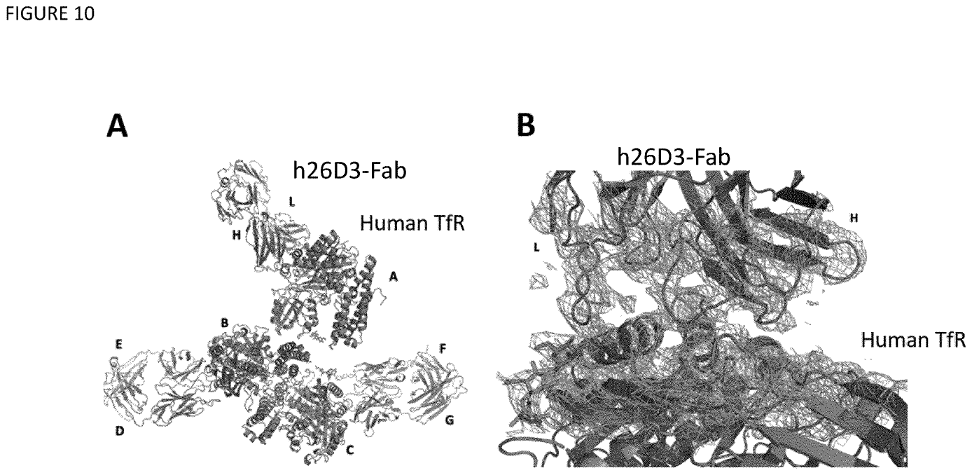

The final, refined structure of the complexes showing the overall folds is depicted in FIG. 10. As shown in FIG. 10A, there were three independent complexes in the asymmetric unit. The chain names as used in the coordinate files are indicated. FIG. 10B shows an example of the electron density contoured at the interface between hTfR1 and heavy/light chain of h26D3-Fab. The protein chains are drawn in cartoon representation while sugar moieties are shown in stick representation. The binding interface interaction between h26D3 and human TfR1 was extracted from the X-ray structure and described in the following to provide information about the precise binding of h26D3 to human TfR1.

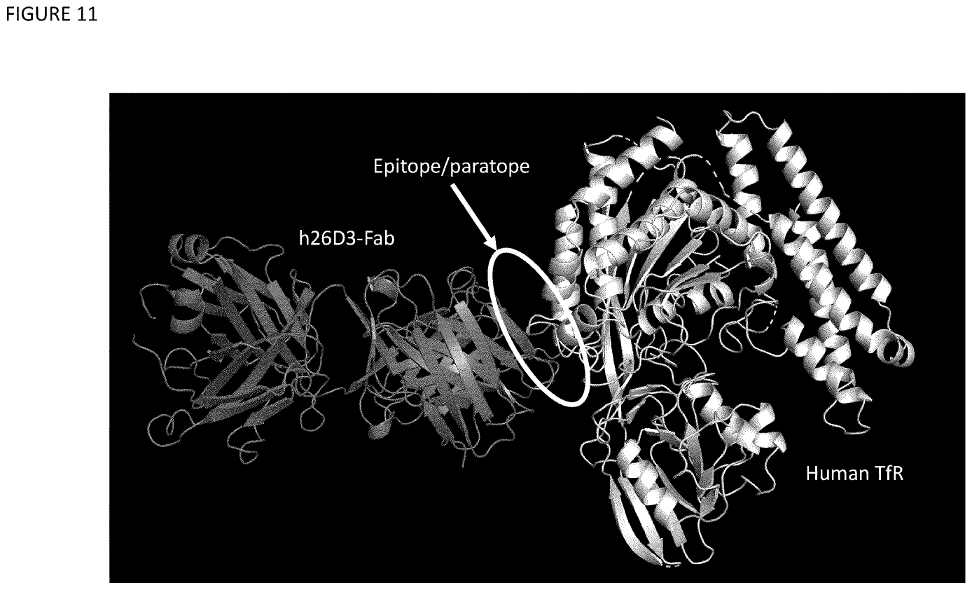

The binding interface between hTfR1 and h26D3-Fab is depicted in FIGS. 10 and 11, and interaction was observed between the amino acid residues indicated in Table 6.

TABLE 6
Amino acid residues involved in interaction
between h26D3 and hTfR1

| | | Human TfR1 |
|---|---|---|
| h26D3 heavy chain | | |
| 50ASP | interacts with | 150Asn |
| 57Thr | interacts with | 150Asn |
| 59Ser | interacts with | 150Asn |
| 105Ser | interacts with | 154Pro |
| 105Ser | interacts with | 159Ser |
| 105Ser | interacts with | 161Lys |
| 106Tyr | interacts with | 161Lys |
| 106Tyr | interacts with | 158Gly |
| 107Tyr | interacts with | 151Ser |
| h26D3 light chain | | |
| 32Ser | interacts with | 163Glu |
| 32Ser | interacts with | 385Lys |
| 33Thr | interacts with | 160Gln |

Table 6 describes the key residues from both sides involved in the epitope/paratope interface as determined from the crystal structure. Additional residues in the vicinity are also likely to be important for the binding between h26D3 and hTfR1. In addition, as described in Example 9 below, several positions outside the observed binding interaction show important participation in binding of h26D3 to hTfR1.

In Table 7 below, the amino acids of human TfR1 that are involved in the respective interactions with h26D3, Ft and Tf are listed. Notably, no amino acids involved in the binding of h26D3 form part of any of the binding interfaces for the endogenous ligands. This illustrates that the binders of the present disclosure, as exemplified by h26D3, bind to hTfR1 outside the binding sites used by Ft and Tf.

TABLE 7
Amino acid residues in hTfR1 which
interact with the respective ligand

| h26D3 | Ferritin* | Transferrin# |
|---|---|---|
| 150Asn | 195Ser | 121Arg |
| 151Ser | 197Gln | 123Tyr |
| 154Pro | 199Ser | 125Asp |
| 158Gly | 201Ile | 126Asp |
| 159Ser | 208Arg | 622Val |
| 161Lys | 209Leu | 623Arg |
| 163Glu | 210Val | 626Asn |
| 385Lys | 212Leu | 629Arg |
| | 215Asn | 640Gln |
| | 343Glu | 643Tyr |
| | 343Lys | 651Arg |
| | 344Lys | 661Gly |
| | 348Asn | 662Asn |
| | 374Lys | 663Ala |
| | | 664Glu |
| | | 667Asp |
| | | 757Asp |
| | | 758Asn |

*Montemiglio et al (2019), Nat Commun 10: 1121
#Eckenroth et al (2011), Proc Natl Acad Sci USA 108: 13089

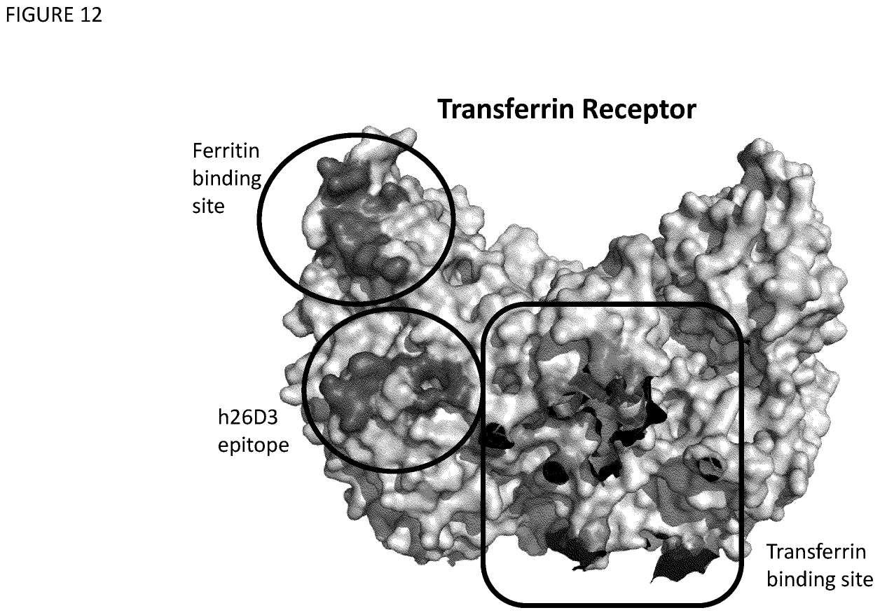

The different epitopes on the hTfR1 structure (pdb: 1SUV) are illustrated further in FIG. 12. As shown in FIG. 12, the Ft binding site is located on the apical domain of hTfR1, the Tf binding site is mainly located on the helical domain of hTfR1 and the h26D3 epitope is located on the protease-like domain of hTfR1. The structure illustrates that the different ligands and binder use distinct, specific surface areas on the hTfR1 structure. hTfR1 is a homodimer having two identical chains, and the epitopes are only indicated on one of these chains.

### Example 6

### Generation and Characterization of hTFR1 Knock-In Mice

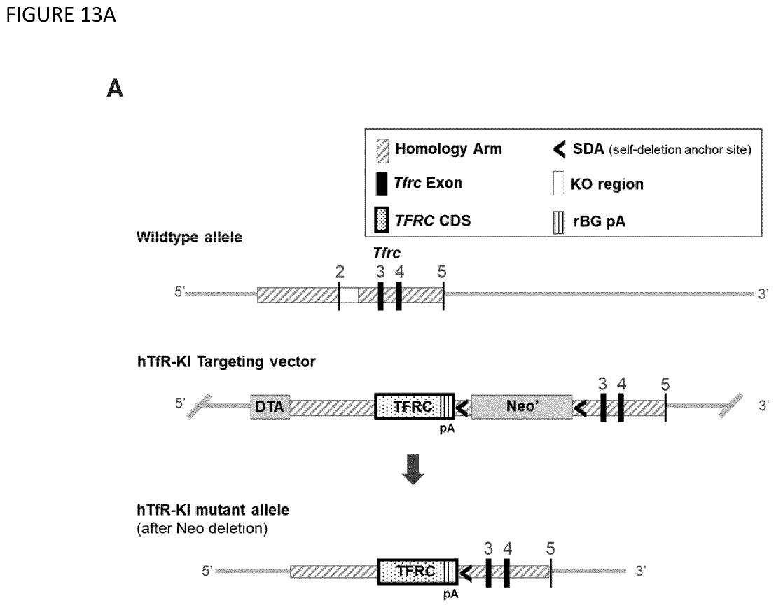

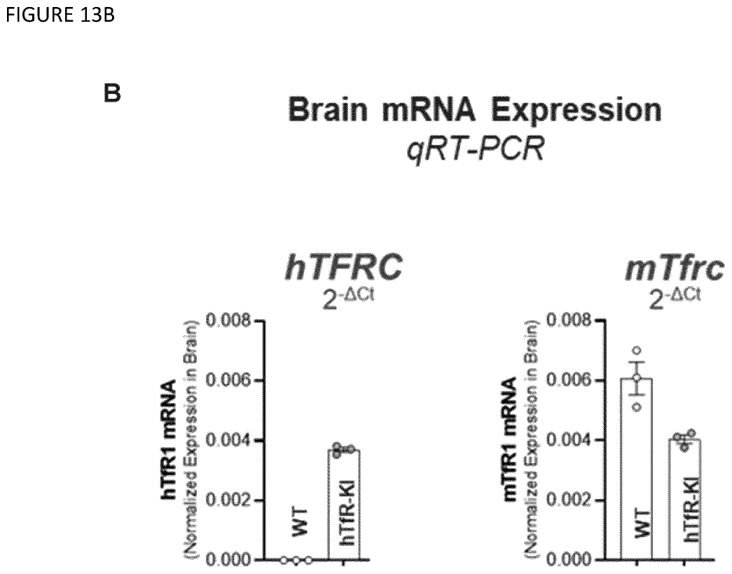

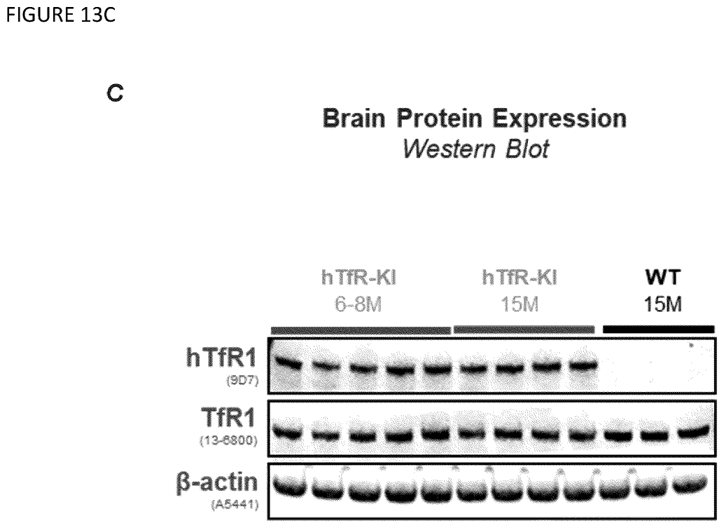

Human TfR1 knock-in (hTfR1-KI; TFR1C-KI) mice were generated by homologous recombination (experimental work performed at Cyagen US). A cDNA vector carrying the TFR1C (NCBI Reference Sequence: NM_001128148.3) ectodomain and murine Tfrc transmembrane and intracellular domain were introduced by pronuclear microinjection in C57BL/6N ES cells Tfrc. The coding region of Tfrc exon 2 plus partial intron 2 were replaced with the TFR1C chimeric cassette (FIG. 13A). Correct insertion of hTfR1 cDNA was verified by Southern blot and PCR. Transgene expression in hTfR1-KI mice was confirmed in brain tissue by qRT-PCR (FIG. 13B) and western blot (FIG. 13C), indicating endogenous expression levels. hTfR1-KI mice were maintained on a C57BL/6N background and only heterozygous hTfR1-KI mice were used for experiments.

### Example 7

### Brain Uptake of hTfR1 Binding Constructs In Vivo

To evaluate hTfR1-mediated brain uptake in vivo, monovalent Fc-scFv constructs (see Example 4) were produced for four different binding proteins. A known binder to hTfR1, 15G11-1, was used as a control (Yu et al (2014), supra). This hTfR1 binder has been described to be active in vivo and is used as a positive reference control for brain uptake. In addition, a construct containing a non-hTfR1 scFv binder based on the anti-amyloid $ antibody mAb158 was designed and included as a negative control in the form of an Fc fusion construct (Fc-scFv158, also referred to as simply “158” here and in the figures). The different Fc-scFv constructs were injected intravenously (i.v.) into hTfR1 knock-in (hTfR1-KI) mice produced as described in Example 6 (n=4 per construct) at equimolar doses of 30 nmol/kg (corresponding to approximately 2.3 mg/kg). Plasma and brain exposure was assessed 24 h after dose.

The animals were anaesthetized using isoflurane and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with a minimum of 50 ml of cold PBS. Following perfusion, brains were extracted and the olfactory bulbs removed. The brains were separated into left and right hemispheres and cerebellum was removed from the left hemisphere, after which the left hemisphere was weighed and snap frozen on dry ice and stored at −80° C. until further preparation and analysis of the concentrations of injected constructs using a Meso Scale Discovery (MSD) based assay. The right hemispheres were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing (see Example 8 below).

For brain concentration measurements, frozen left hemispheres were thawed on ice and homogenized in TBS by automated bead homogenization. Triton was added to the homogenate to a final Triton concentration of 0.5% before centrifugation at 16 000×g, after which supernatants were collected.

Brain and plasma concentrations of anti-hTfR1 Fc-scFv were determined using a custom build MSD assay detecting the human Fc. A standard 96-well MSD plate (MSD, #L15XA-3) was coated with 0.5 μg/ml goat anti-human IgG, Fcγ fragment specific antibody (Jackson Immuno Research Europe Ltd, #109-005-098) diluted in 1×PBS (Medicago AB, #09-9400-100). After incubation at 4° C. overnight, the plate was washed 4× in 1×PBS-TWEEN (Fisher Scientific, #09-9410-100) and blocked with 150 μl 1% BlockerA in PBS-TWEEN (MSD, #R93BA-4) per well. Samples and corresponding standards, ranging from 400 pM to 0.1 pM in 1:4 dilution steps, were added and incubated for 2 h and 900 rpm at room temperature. A 1 h incubation step with mouse anti-human IgG (Mabtech, 3850 January 1000, MT145) diluted to 0.5 μg/ml was included, followed by 1 h incubation of SULFO-TAG conjugated anti-mouse antibody (MSD, R32AC-1) diluted to 0.5 μg/ml when the plate was incubated for another hour at room temperature and 900 rpm. 150 μl MSD read buffer (MSD, #R92TC) per well was added before reading the plates in an MSD SECTOR Imager. Between each incubation step, a 4× wash in 1×PBS-TWEEN was performed. All antibodies and samples, except the coating antibody, were diluted in 1% Blocker A in PBS-TWEEN and added in a volume of 50 μl/well. The concentration of the analytes in the samples were evaluated with the MSD workbench software, using a 4PL curve fitting algorithm and curve weighting 1/Y2 for the standard curve. Statistical analysis was performed in GraphPad Prism (v. 9.0.0) using one way ANOVA with Tukey's post hoc test.

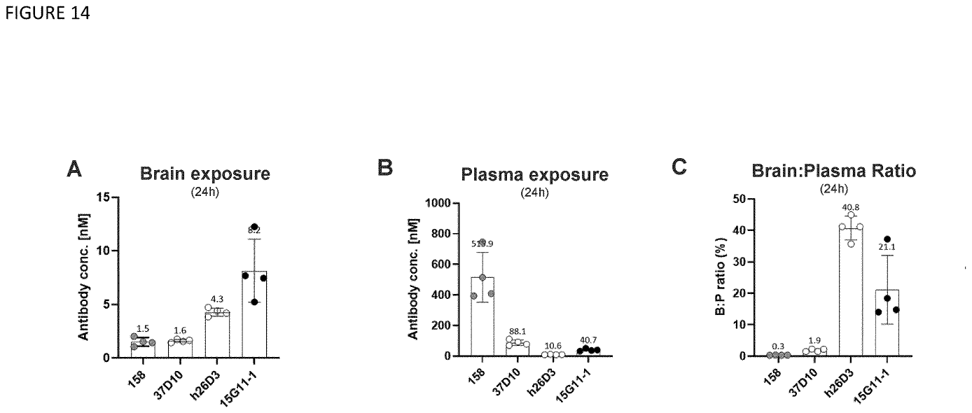

The results are shown in FIG. 14. As shown in FIG. 14A, substantially higher brain concentrations were observed for the two test constructs and the positive control 15G11-1, compared to the negative control (158) at 24 h after dose. As shown in FIG. 14B, the plasma concentrations of the two test constructs and the positive control 15G11-1 were lower at 24 h compared to that of 158, indicating that hTfR1 engagement leads to a faster plasma clearance. The brain-to-plasma concentration ratios are shown in FIG. 14C. The two test constructs and the positive control 15G11-1 showed a significantly enhanced brain exposure relative to plasma in comparison to the negative control. Taken together, the data supports hTfR1-mediated BBB transport in this experiment for the tested, novel hTfR1 binders.

### Example 8

### Immunohistochemistry Data on Brain Exposure

In vivo engagement of hTfR1 by the Fc-scFv construct was studied further using a qualitative immunohistochemistry (IHC) analysis. In brief, coronal brain sections at a thickness of 20 μm were obtained from PBS-perfused brain hemispheres of the mice described in Example 7 using a cryostat (Microm NX50 CryoStar, Epredia). The sections were collected on Superfrost plus slides (Menzel-Glaser, #J1800AMNZ) and air-dried prior to IHC. The brain sections were washed with PBS (pH 7.4) for 15 min and incubated in blocking buffer (5% BSA, 0.25% Triton-X in PBS) for 2 h at room temperature. To visualize i.v. dosed constructs, brain sections were incubated with a secondary goat anti-human IgG (heavy and light chain specific) conjugated to Alexa Fluor 488 (Invitrogen, #A11013) for 120 min at room temperature followed by 3×15 min wash in PBS. Slides were mounted with Fluoromount-G (Invitrogen, #00-4958-02) for imaging analysis. Confocal images from cerebral cortex were captured using a Leica Stellaris 5 confocal system equipped with a HC PL APO 40×/1.25 GLYC motCORR CS2 objective (Leica, #11506423).

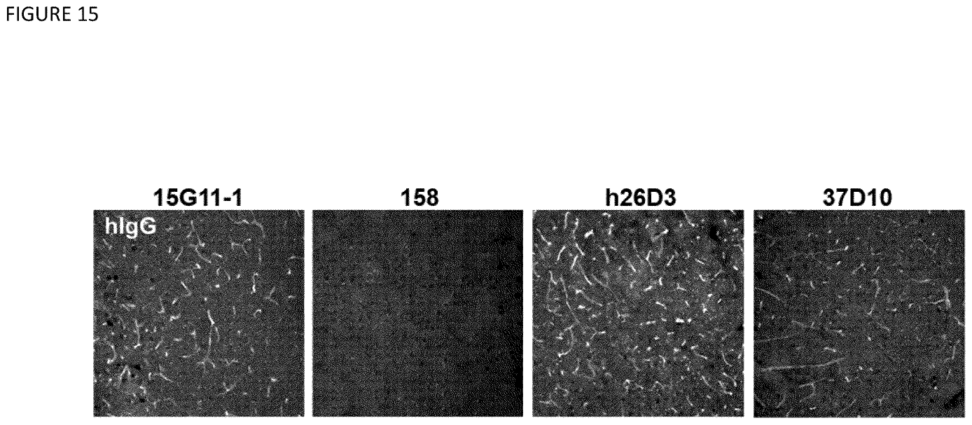

Distinct IHC immunofluorescence signals were observed in brain capillaries with positive reference module 15G11-1, while a minimal IHC signal was detected in brain sections from mice injected with negative control 158 (FIG. 15). Brain capillary IHC signal was observed for the two test constructs h26D3 and 37D10, of which h26D3 showed the strongest immunofluorescence signal, comparable to the positive control 15G11-1. Taken together, the MSD (Example 7) and IHC (this Example) analyses demonstrate that the hTfR1 binders of the disclosure in a scFv format exhibit an increased brain exposure in hTfR1-KI mice.

### Example 9

### Generation of Affinity Variants and Affinity Determinations

Several variants of the parental antibody h26D3 were generated by substituting tyrosine, tryptophan and aspartic acid residues in the CDRs one by one for alanine residues. The resulting variant VH regions were denoted HC1-HC13 and their amino acid sequences are provided in the sequence listing as SEQ ID NO:45-57, respectively. Variant CDR sequences comprised in these variant VH regions are listed as SEQ ID NO:16-28, respectively. The resulting variant VL regions were denoted LC1-LC6 and their amino acid sequences are provided in the sequence listing as SEQ ID NO:59-64, respectively. Variant CDR sequences comprised in these variant VL regions are listed as SEQ ID NO:29-33, respectively. Table 8 below provides a summary of the specific mutations in each of the alanine variants.

TABLE 8
Alanine substitution variants of VH and VL of h26D3

| Variant | Mutation | Variant | Mutation | Variant | Mutation |
|---|---|---|---|---|---|
| LC1 | Y31A | HC1 | D31A | HC8 | Y60A |
| LC2 | Y38A | HC2 | Y32A | HC9 | Y101A |
| LC3 | Y55A | HC3 | D35A | HC10 | Y106A |
| LC4 | W56A | HC4 | D50A | HC11 | Y107A |
| LC5 | Y97A | HC5 | D54A | HC12 | D111A |
| LC6 | Y100A | HC6 | Y55A | HC13 | Y112A |
| | | HC7 | D56A | | |

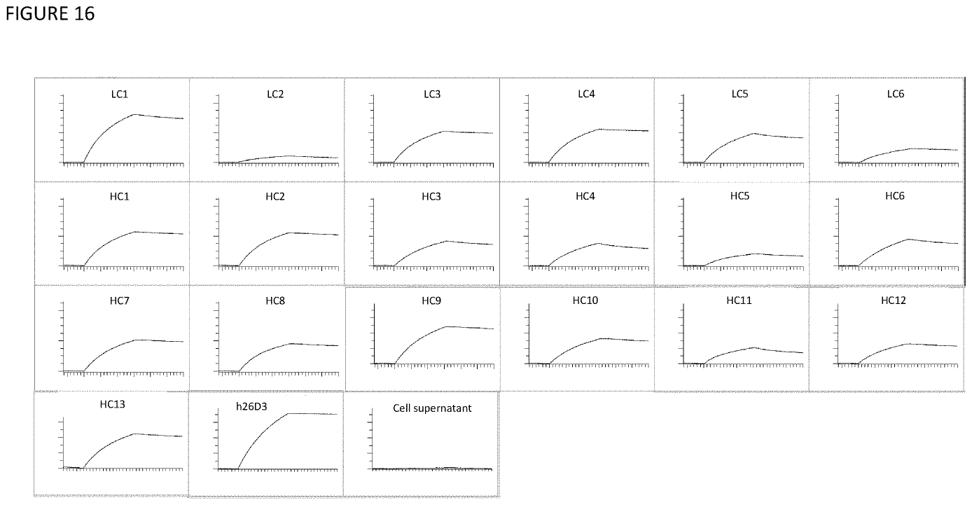

The generated alanine variants were expressed as single mutant, His-tagged Fabs by transient transfection of Chinese hamster ovary cells (ExpiCHO; Thermo Fisher Scientific) according to the manufacturer's instructions. Clarified media, into which the Fabs had been secreted, was used to assess binding to hTfR1 by BLI (Octet RED384, ForteBio). The expressed Fabs were loaded from the cell supernatants onto anti-Fab biosensors during 240 s. Thereafter, association of ectodomain of hTfR1 (SEQ ID NO:74), diluted to 3.75 μg/ml in 1× Kinetics buffer (ForteBio), to the loaded sensors was measured for 300 s, followed by dissociation for 300 s. All variants were confirmed to bind hTfR1 but were affected to different extent (FIG. 16).

Variants showing affected binding to hTfR1 in the screen were selected for further characterization. In addition, double mutants were generated by combining heavy and light chains with alanine substitutions. Table 9 below provides a summary of the specific mutations in each of the alanine variants that were selected.

TABLE 9
Variants of VH and VL of h26D3 selected
for further characterization

| Variant | Mutation VL | Mutation VH |
|---|---|---|
| LC1 | Y31A | None |
| LC5 | Y97A | None |
| LC6 | Y100A | None |
| HC2 | None | Y32A |
| HC3 | None | D35A |
| HC4 | None | D50A |
| HC5 | None | D54A |
| HC6 | None | Y55A |
| HC8 | None | Y60A |
| HC10 | None | Y106A |
| HC11 | None | Y107A |
| HC3/LC1 | Y31A | D35A |
| HC3/LC5 | Y97A | D35A |
| HC6/LC1 | Y31A | Y55A |
| HC6/LC5 | Y97A | Y55A |
| HC8/LC1 | Y31A | Y60A |
| HC8/LC5 | Y97A | Y60A |
| HC10/LC1 | Y31A | Y106A |
| HC10/LC5 | Y97A | Y106A |

The selected variants were expressed as His-tagged Fabs by transient transfection of Chinese hamster ovary cells (ExpiCHO; Thermo Fisher Scientific) according to the manufacturer's instructions. The Fabs were purified at small scale with HisPur™ Ni-NTA Magnetic Beads (Thermo Scientific) according to the manufacturer's instructions followed by buffer exchange into DPBS pH 7.4. Selected variants were also purified at a larger scale by application on a HisTrap Excel column (Cytiva), which was washed with 20 mM Tris, 200 mM NaCl and 5 mM imidazole. The proteins were eluted with 20 mM Tris, 200 mM NaCl and 500 mM imidazole, followed by buffer exchange to DPBS pH 7.4 using a HiPrep 26/10 Desalting column (Cytiva). The proteins were concentrated using an Amicon Ultra centrifugal concentrator (30 MWCO; Millipore). Selected variants were further polished by size exclusion chromatography (SEC; HiLoad 26/600 Superdex 200; Cytiva) in DPBS pH 7.4. Analytical characterization of the protein was done by UV protein determination, SDS-PAGE and HPLC-SEC.

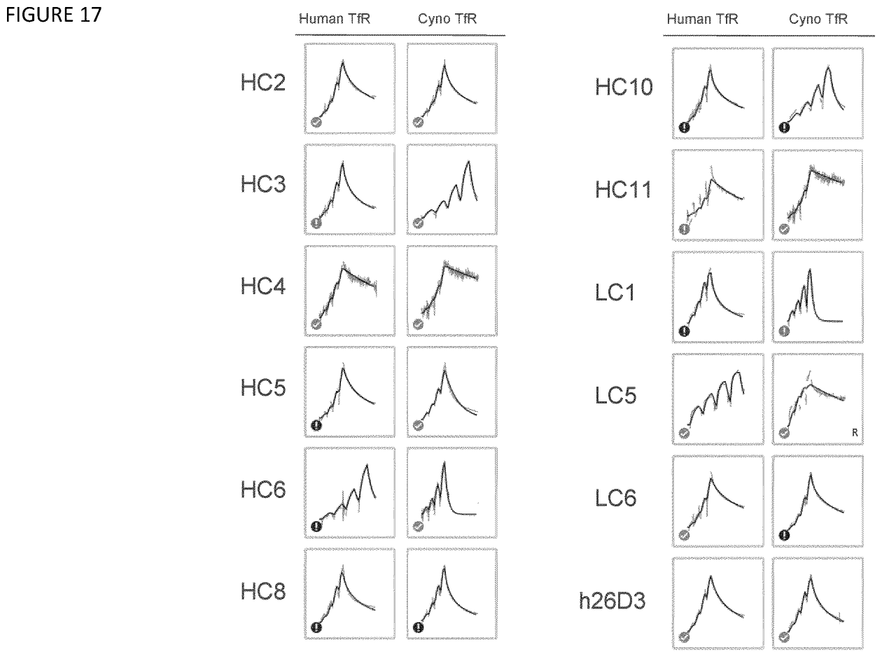

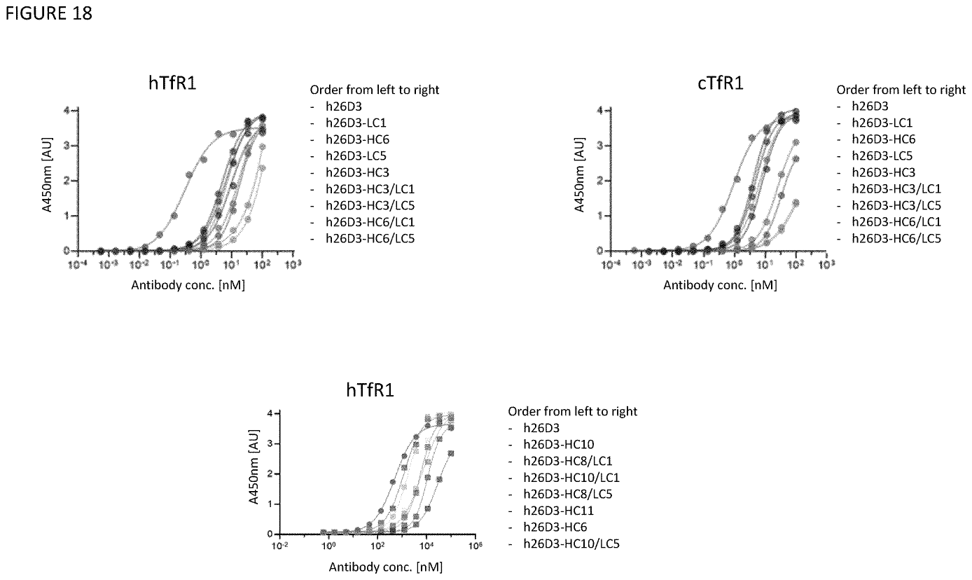

Binding of the purified Fabs to human and cynomolgus TfR1 was evaluated using either SPR (FIG. 17) or indirect ELISA (FIG. 18). For SPR, a Biacore 8K instrument (Cytiva) was used. 1 μg/ml of hTfR1 (SEQ ID NO:86) or cTfR1 (SEQ ID NO:87) was immobilized on a Cm5 sensor chip (Cytiva, #BR100399) using the amine coupling kit type 2 (Cytiva, #BR100633) according to the manufacturer's instruction. The Fabs were injected over the chip using a 2-fold dilution series in four steps starting at 100 nM. The interaction was measured using the single cycle kinetics method with a contact time of 120 s at a flow rate of 30 μl/min followed by a dissociation time of 1000 s. Regeneration of the surface between cycles was done by injecting 3M MgCl₂. The binding data was fitted to a 1:1 interaction model. The Fabs were diluted in HBS-EP+ (Cytiva, #BR100669). Experiments were performed at 25° C. The results are shown in FIG. 17, and the calculated K_{D} values are given in Table 10 below.

TABLE 10
SPR analysis of variant h26D3 Fabs vs. hTfR1 and cTfR1

| Variant | hTfR1 | cTfR1 |
|---|---|---|
| h26D3 Fab | K_{D} (nM) | K_{D} (nM) |
| HC2 | 8.7 | 12 |
| HC3 | 16 | 168 |
| HC4 | 5.1 | 8 |
| HC5 | 20 | 39 |
| HC6 | 156 | 164 |
| HC8 | 10 | 19 |
| HC10 | 9.2 | 4.6 |
| HC11 | 388 | 2.7 |
| LC1 | 14 | 70 |
| LC5 | 240 | ND |
| LC6 | 8.5 | 9.4 |
| h26D3 | 10 | 12 |

For the indirect ELISA, half area 96-well plates (Corning, #3690) were coated with 1 μg/ml recombinant ectodomain of hTfR1 (SEQ ID NO:74) in PBS overnight at 4° C. The coated plates were blocked using Pierce protein-free blocking solution (Thermo Fisher Scientific, #37572) for 1 h at room temperature with shaking and washed four times in PBS containing 0.1% TWEEN-20. Serial dilutions (1:3) of various expressed constructs in incubation buffer (1% BSA, 0.1% TWEEN-20 in PBS) were incubated for 1 h at room temperature. Following the four wash steps, bound test constructs were detected by addition of anti-human-IgG F(ab′)₂-HRP antibody (Jackson Immuno Research, #109-036-003) at 1:5000 dilution in incubation buffer (1 h, room temperature). Following four wash steps, K-Blue® Aqueous TMB substrate (Neogen, #331177) was added to the wells for 15 min at room temperature before the reaction was stopped with 1:1 dilution of 0.5 M H₂SO₄. The optical density at 450 nm was recorded (Spark, Tecan) and background signal was subtracted before analysis. The obtained results are shown in FIG. 18.

Based on the Biacore and ELISA measurements, several variants were identified within a wide range of affinities for human TfR1. Many variants exhibited a retained cross-reactivity to cynomolgus TfR1.

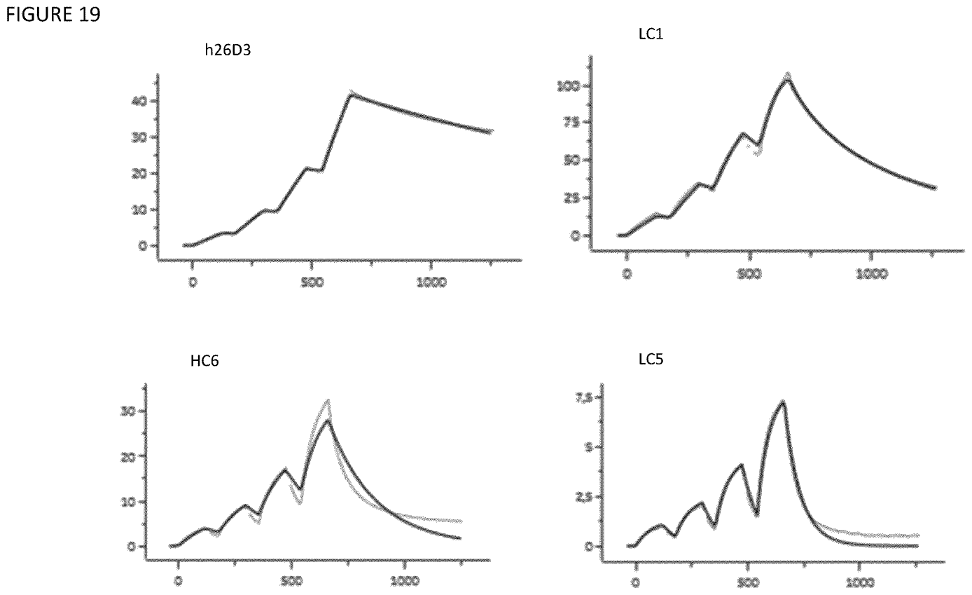

Finally, selected variants were reformatted to scFv and used in the context of the bispecific binding molecule format disclosed in WO2022/258841. Bispecific binding molecules comprising scFv modules constructed from h26D3 and selected alanine mutants were expressed in ExpiCHO cells as described above. Filtered supernatants were applied to a MabSelect SuRe column (Cytiva) which was subsequently washed with DPBS pH 7.4. Expressed binding molecules were eluted by application of 0.7% HAc pH 2.5, followed by immediate neutralization of the sample to pH 7.5. Purified samples were polished further by subjecting them to size exclusion chromatography (SEC; HiLoad 26/600 Superdex 200; Cytiva) in DPBS pH 7.4. The purified constructs were concentrated using centrifugal concentrators Amicon Ultra (30 MWCO, Millipore). Each purified expressed construct was characterized using SDS-PAGE, size-exclusion chromatography (Superdex 200 Increase 3.2/300; Cytiva) and UV protein determination. Binding to hTfR1 was evaluated using SPR as described above with adjustments of the concentration interval depending on the variant. As shown in FIG. 19 and in Table 11 below, the different tested variants exhibited a range of affinities for the hTfR1 target.

TABLE 11
SPR analysis of variant h26D3 scFv in bispecific format vs. hTfR1

| Variant | K_{D} vs hTfR1 (nM) |
|---|---|
| h26D3 | ~10 |
| LC1 | ~40 |
| HC6 | ~100-200 |
| LC5 | ~400-500 |

### Example 10

### Design of Gen 2A Bispecific Binding Proteins with “VH First” or “VL First” scFv Modules

Fourteen different bispecific binding protein constructs were designed using the “Gen 2A” format originally described in WO2022/258841 (FIG. 4, left panel), using the scFv format of hTfR1 binder h26D3 HC6 described above in two different configurations, “VH first” as represented by SEQ ID NO:88 and “VL first” as represented by SEQ ID NO:89. Seven of the constructs were designed using the “VH first” configuration (#1-7, having single chain components represented by SEQ ID NO:90-96, respectively), whereas another seven of the constructs were designed using the “VL first” configuration (#8-14, having single chain components represented by SEQ ID NO:97-103, respectively). The antibody heavy chain used in all of these constructs was the same and is represented by SEQ ID NO:104.

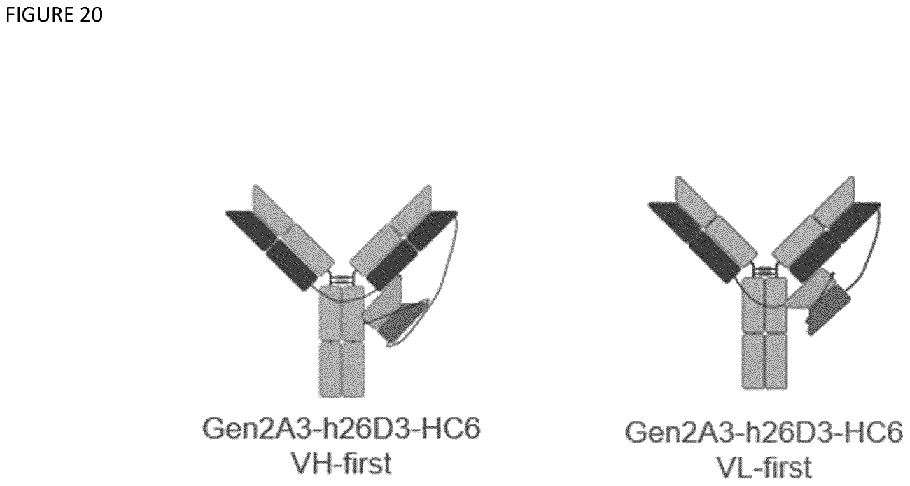

FIG. 20 provides a schematic overview of the different tested constructs. Constructs #1-7 (VH first) and #8-14 (VL first) were produced as a series of different combinations of linker lengths as shown in Table 12, in order to investigate the influence of linker length and VH vs. VL first configuration in combination with the h26D3 HC6 scFv binder on the binding to hTfR1 and the positioning of the antibody when hTfR1 is expressed on the cell surface.

TABLE 12
Linker properties for tested constructs on the Gen 2A format

| | | | | | | |
|---|---|---|---|---|---|---|
| | | Measured | | Measured | | Total |
| | | necessary | | necessary | Total | measured |
| | Linker | linker | Linker | linker | linker | necessary |
| Construct | 1 | length | 2 | length | size | size |
| # | [L1] | [L1] (Å) | [L2] | [L2] (Å) | (Å) | (Å) |

VH first configuration

| | | | | | | |
|---|---|---|---|---|---|---|
| 2A-3 #1 | 1x G₄S | ≈44 | 4x G₄S | ≈40 | ≈95 | ≈88 |
| 2A-3 #2 | 2x G₄S | | 4x G₄S | | ≈114 | ≈88 |
| 2A-3 #3 | 3x G₄S | | 4x G₄S | | ≈133 | ≈88 |
| 2A-3 #4 | 1x G₄S | | 5x G₄S | | ≈114 | ≈88 |
| 2A-3 #5 | 2x G₄S | | 5x G₄S | | ≈133 | ≈88 |
| 2A-3 #6 | 3x G₄S | | 5x G₄S | | ≈154 | ≈88 |
| 2A-3 #7 | 5x G₄S | | 5x G₄S | | ≈192 | ≈88 |

VL first configuration

| | | | | | | |
|---|---|---|---|---|---|---|
| 2A-3 #8 | 1x G₄S | ≈46 | 4x G₄S | ≈60 | ≈95 | ≈106 |
| 2A-3 #9 | 2x G₄S | | 4x G₄S | | ≈114 | ≈106 |
| 2A-3 #10 | 3x G₄S | | 4x G₄S | | ≈133 | ≈106 |
| 2A-3 #11 | 1x G₄S | | 5x G₄S | | ≈114 | ≈106 |
| 2A-3 #12 | 2x G₄S | | 5x G₄S | | ≈133 | ≈106 |
| 2A-3 #13 | 3x G₄S | | 5x G₄S | | ≈154 | ≈106 |
| 2A-3 #14 | 5x G₄S | | 5x G₄S | | ≈192 | ≈106 |

### Example 11

### Production and Purification of Designed Gen 2A Constructs

The fourteen constructs designed as described in Example 10 were functionally expressed by transient transfection of Chinese hamster ovary cells (ExpiCHO; Thermo Fisher Scientific) according to the manufacturer's instructions. Filtered cell culture supernatants were applied to a MabSelect SuRe column (Cytiva), which was subsequently washed with DPBS pH 7.4. Expressed binding proteins were eluted by application of 0.7% HAc pH 2.5, followed by neutralization of the sample to pH 7.5. Purified samples were polished further by size exclusion chromatography (SEC; HiLoad 26/600 Superdex 200; Cytiva) in DPBS pH 7.4. Each purified expressed construct was characterized using SDS-PAGE, size-exclusion chromatography (Superdex 200 Increase 3.2/300; Cytiva) and UV protein concentration determination.

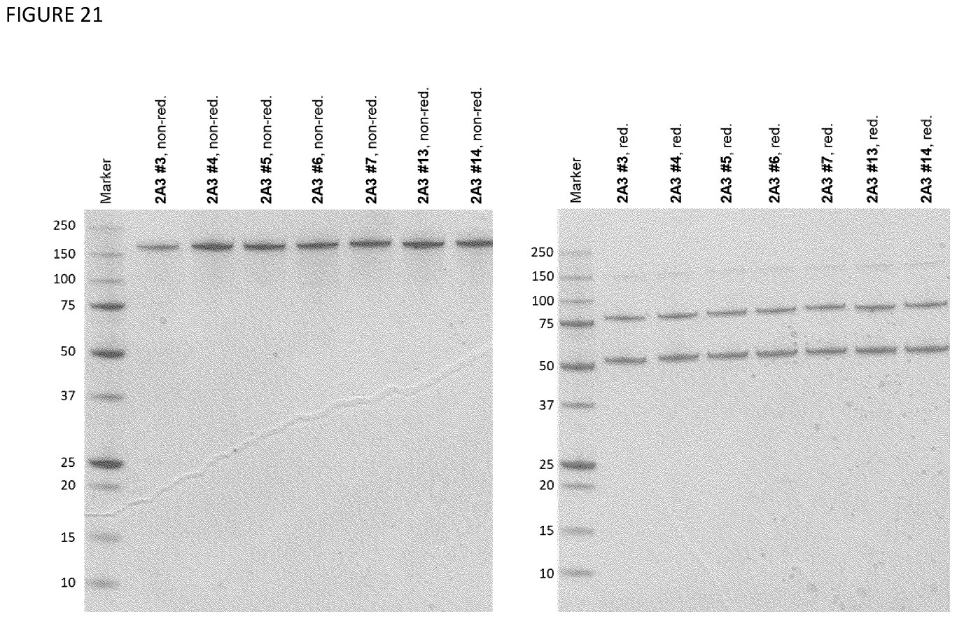

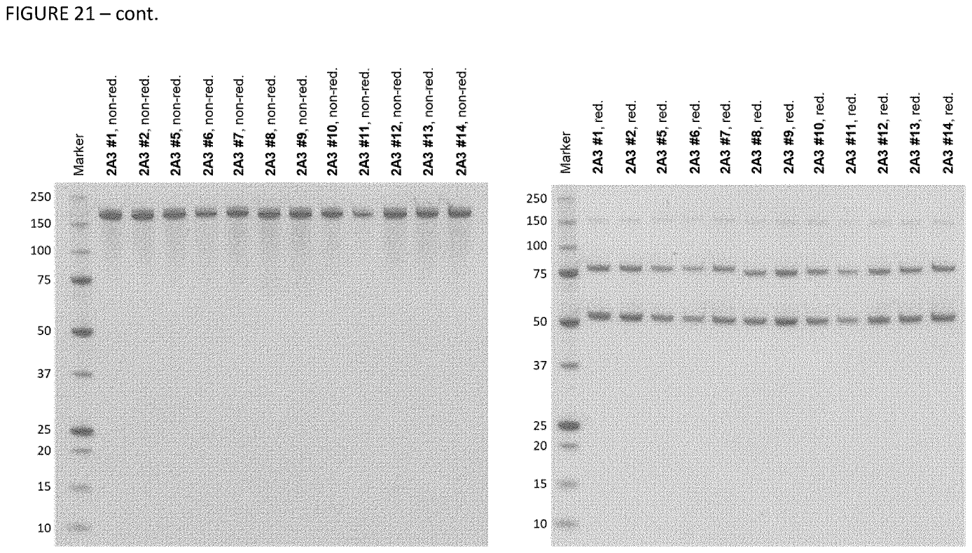

Results of the purification are given in Table 13. A representative SDS-PAGE analysis of purified constructs is shown in FIG. 21. The non-reduced gel showed one band at around 175 kDa. The reduced gel showed the expected two-band profile for the Gen 2A format, with the antibody heavy chain at around 50 kDa and the single chain component comprising two light chains linked to the hTfR1 binding scFv at an approximately molecular weight of 75 kDa. As shown in Table 13, the monomeric content of the bispecific binding proteins was high (in general >98%) and they were produced at low mg/I levels.

TABLE 13
Purification of test constructs

| | | % | Quantity | Yield |
|---|---|---|---|---|
| Construct # | Batch | monomer | (mg) | (mg/l culture) |
| 2A-3 #1 | 220909 | 96.3 | 1.0 | 2.8 |
| 2A-3 #2 | 220915 | 96.5 | 5.4 | 14.3 |
| 2A-3 #3 | 220811 | 99.8 | 1.6 | 8.2 |
| 2A-3 #4 | 220811 | 97.3 | 2.8 | 13.8 |
| 2A-3 #5 | 220812 | 98.6 | 1.1 | 5.3 |
| 2A-3 #5 | 220915 | 99.5 | 0.5 | 1.2 |
| 2A-3 #6 | 220812 | 99.2 | 0.4 | 2.2 |
| 2A-3 #6 | 220915 | 99.9 | 0.1 | 0.3 |
| 2A-3 #7 | 220829 | 99.9 | 0.5 | 2.6 |
| 2A-3 #7 | 220913 | 99.9 | 1.0 | 2.4 |
| 2A-3 #8 | 220915 | 97.9 | 0.2 | 0.5 |
| 2A-3 #9 | 220826 | 99.6 | 3.2 | 8.5 |
| 2A-3 #9 | 220915 | 99.9 | 0.5 | 1.3 |
| 2A-3 #10 | 220913 | 99.1 | 6.7 | 15.8 |
| 2A-3 #11 | 220916 | 93.6 | 0.6 | 1.5 |
| 2A-3 #12 | 220913 | 93.0 | 1.2 | 3.3 |
| 2A-3 #13 | 220829 | 99.9 | 0.7 | 3.6 |
| 2A-3 #13 | 220913 | 99.3 | 0.6 | 1.7 |
| 2A-3 #14 | 220829 | 99.7 | 0.7 | 3.6 |
| 2A-3 #14 | 220913 | 99.8 | 0.8 | 2.0 |

### Example 12

### Analysis of Gen 2A Binding to hTfR1 Using SPR

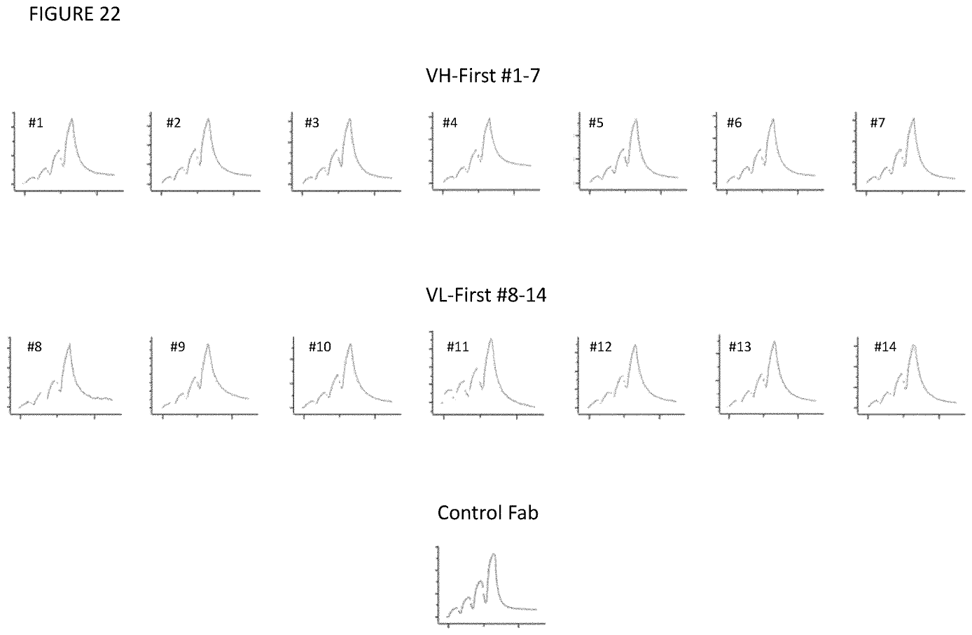

Binding to hTfR1 of the bispecific binding proteins, expressed and purified as described in Example 11, was evaluated using SPR (Biacore 8K, Cytiva). 2 μg/ml of hTfR1 was immobilized on a Cm5 sensor chip (Cytiva, #BR100399) using the amine coupling kit type 2 (Cytiva, #BR100633) according to the manufacturer's instruction. Bispecific binding proteins were injected over the chip using a 2-fold dilution series in four steps starting at 200 nM. The interaction was measured using the single cycle kinetics method with a contact time of 120 s at a flow rate of 30 μl/min followed by a dissociation time of 600 s. Regeneration of the surface between cycles was done by injecting 3 M MgCl₂. The binding data was fitted to a 1:1 interaction model. The bispecific binding proteins were diluted in HBS-EP+ (Cytiva, #BR100669). Experiments were performed at 25° C. The data in FIG. 22 shows that all designed and produced Gen 2A constructs bind to hTfR1. All constructs show similar on-rates and off-rates compared to a control Fab construct of the hTfR1 binder h26D3 HC6. This illustrates that all the expressed constructs are functional and that neither linker lengths nor “VH first”/“VL first” configuration directly influence the binding of the constructs to hTfR1.

### Example 13

### Binding to hTfR1 Expressed on Cell Surfaces

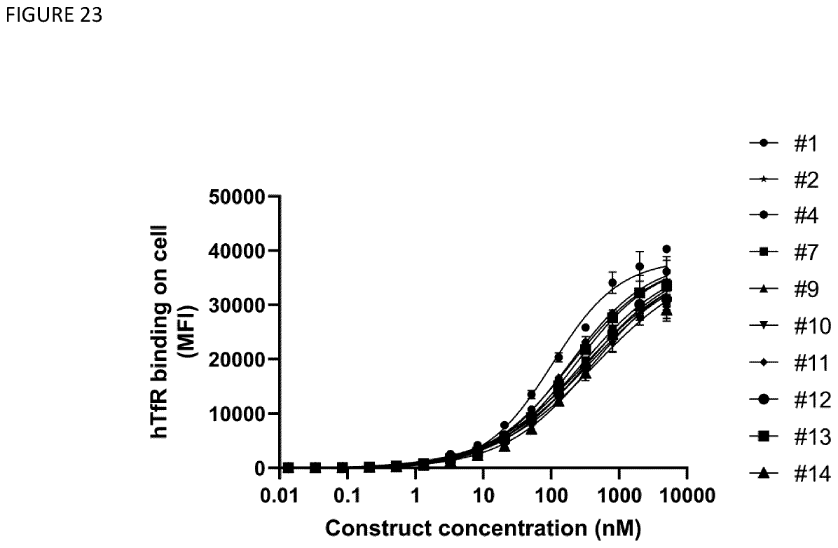

Binding to hTfR1 on cells was measured on the immortalized human B lymphocyte cell line Ramos (Sigma, cat: 85030802). This cell line is known for expressing high levels of hTfR1 on the cell surface. Fcγ receptors were blocked using Fc receptor blocker (Innovex biosciences, #NB309-4X-40) for 30 min at 4° C., after which the cells were washed in PBS. The cells were seeded in a 96 well V bottom plate (#249570, Thermo Scientific Nunc), and serially diluted Gen 2A constructs were added and the plate was incubated at 4° C. overnight. The cells were washed with PBS with 1% BSA and then fixed with freshly prepared 4% formaldehyde (Thermo Scientific Pierce, #28906) diluted in PBS for 15 min at room temperature. The cells were washed with PBS containing 1% BSA, and then stained. The bispecific binding proteins bound to hTfR1 on the cell surfaces were detected through their shared IgG heavy chain component using a fluorescently labeled secondary goat F(ab′)₂ anti-human IgG(γ)-Alexa fluor 488 (Invitrogen Life technologies, #H10120). Staining was carried out for 30 min at 4° C. After incubation with the detection reagent, cells were washed with PBS containing 1% BSA. The cells were finally resuspended in 200 μl PBS with 1% BSA and acquired using a BD FACSLyric flow cytometer system (BD Biosciences). Samples were analyzed using flowJo software (BD Biosciences). The measured median fluorescence intensities (MFI) were plotted against binding protein concentration and displayed in FIG. 23. The results show that all tested Gen 2A constructs regardless of linker length and “VH first”/“VL first” configuration bind similarly to hTfR1 expressed on cell surfaces.

### Example 14

### Complement Dependent Cytotoxicity (CDC) Analysis

Complement activity is initiated by C1q binding to an Fc part of e.g. an antibody, leading further to binding of other complement factors, ultimately leading to cell death. To evaluate whether a given bispecific test construct gives rise to any CDC activity by enabling C1q binding to Fc, Ramos cells (Sigma, cat: 85030802) were used as target cells for CDC analysis. To measure cell death, Ramos cells were labeled with cell viability dye, Calcein-AM (Sigma, 17783). These labeled cells were then treated with serially diluted bispecific test constructs in the presence of pooled human complement serum (Innovative Research Inc, #39337) for 4 h at 37° C., 5% CO₂. As negative control, cells were treated with a similar concentration of bispecific constructs in the presence of C1q depleted human serum (Sigma, #234401). As positive control, the monoclonal antibody rituximab (MabThera; Roche) was also tested under both conditions (pooled complement serum and C1q depleted serum).

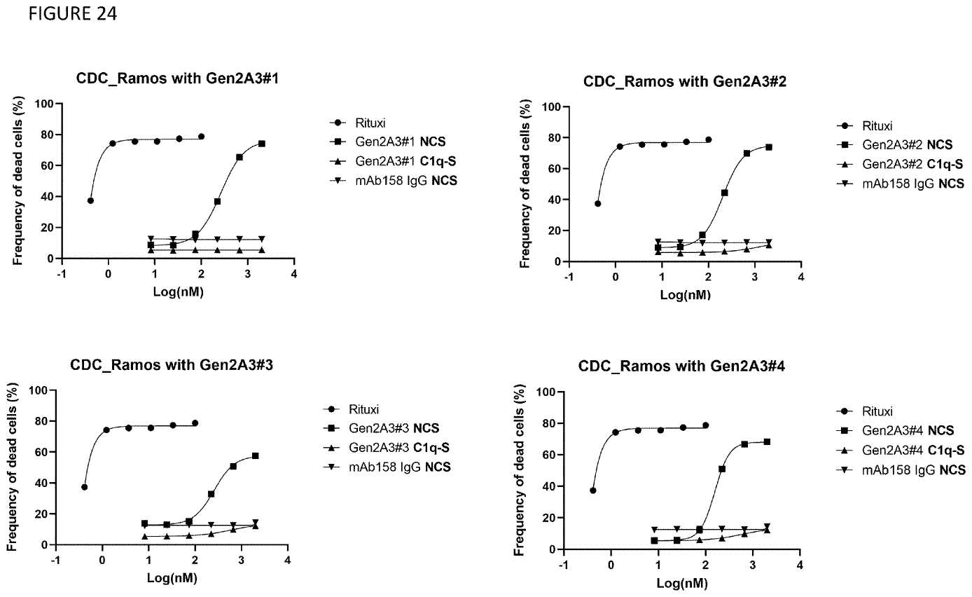

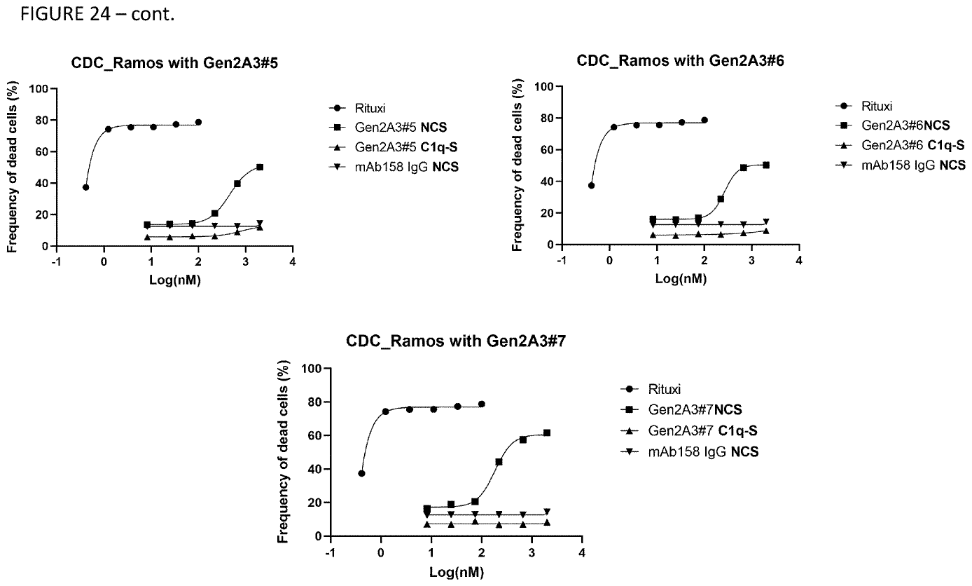

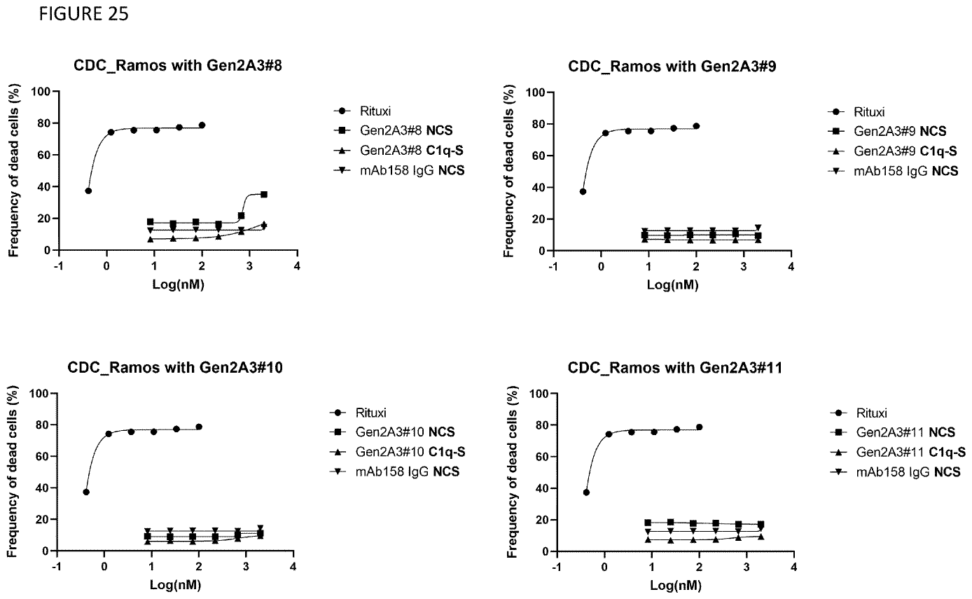

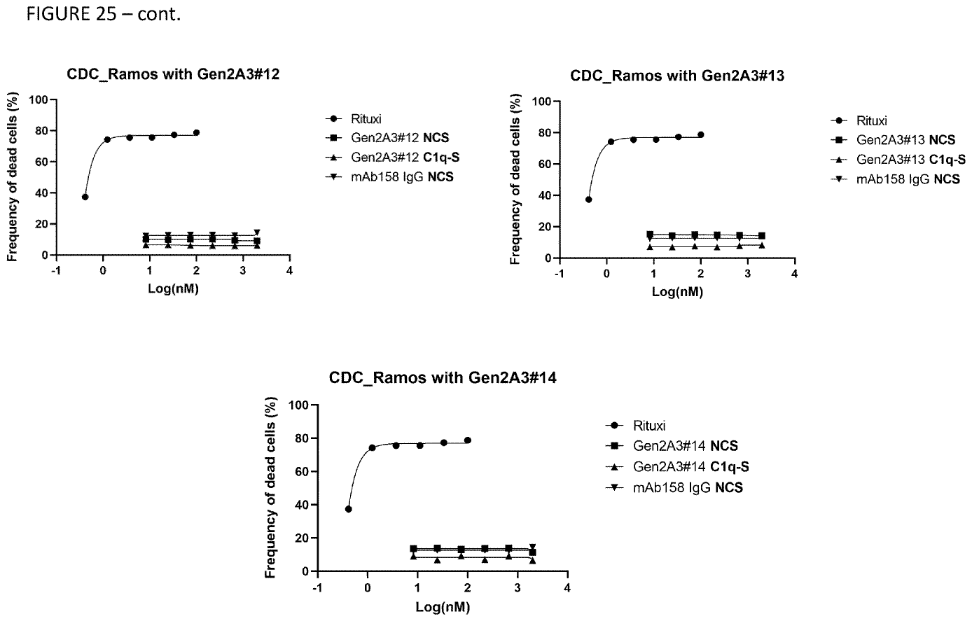

Treated cells were acquired using a BD BDLyric flow cytometer (BD Biosciences). Samples were analyzed using flowJo software (BD Biosciences). The frequency of cell death was determined on calcein AM quenched gated cells and plotted against concentration of the tested bispecific construct or control. The results are shown in FIG. 24 for constructs #1-7 (“VH first”) and in FIG. 25 for constructs #8-14 (“VL first”). It was clearly observed that the bispecific binding proteins with the “VH first” configuration mediated CDC activity, leading to cell death (FIG. 24). On the other hand, none of the bispecific binding proteins in the “VL first” configuration mediated any CDC activity (FIG. 25), leading to the conclusion that the hTfR1 binding mediated by the “VL first” configuration inhibits C1q binding to the Fc domain of the antibody.

### Example 15

### Plasma and Brain Exposure of Gen2A Constructs with hTfR1 Affinity Variants

To evaluate brain and plasma exposure over time of bispecific binding protein constructs according to the disclosure, additional constructs were generated based on the Gen 2A format, in analogy to Example 10. This example investigates the antibody mAb158 in a hIgG1 format with the Fc mutation K322A, with or without three affinity variants of the hTfR1 binding module h26D3 (see Example 9). The tested constructs and their amino acid sequences are given in Table 14.

TABLE 14
Test constructs and amino acid sequences

| Designation | Heavy chain | Light chain |
|---|---|---|
| mAb158 hIgG1-K322A | SEQ ID NO: 110 | SEQ ID NO: 108 |
| 2A3#2-LC1-K322A | SEQ ID NO: 110 | SEQ ID NO: 111 |
| 2A3#2-HC6-K322A | SEQ ID NO: 110 | SEQ ID NO: 70 |
| 2A3#2-LC5-K322A | SEQ ID NO: 110 | SEQ ID NO: 91 |

The different affinity variants as well as comparator mAb158 hIgG1 were injected intravenously (i.v.) into hTfR1 knock-in (hTfR1-KI) mice, generated as described in Example 6 (n=15 per test item) at equimolar doses of 40 nmol/kg (corresponding to approximately 6-7 mg/kg). Exposure in plasma and brain was assessed at five consecutive termination timepoints of 4, 24, 72, 168 and 240 h respectively, with n=3 mice per timepoint and test compound. Blood for the assessment of continuous plasma concentration versus time profiles was sampled at 0.25, 4, 24, 48, 72, 120, 168 and 240 h after administration from the group of animals (n=3) sacrificed at the 240 h timepoint. In-life blood was sampled from the saphenous vein into Sarstedt Microvette CB300 K2E tubes.

At the individual termination timepoints, animals were deeply anaesthetized using isoflurane, and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with ice cold PBS. Following perfusion, brains were extracted, and the olfactory bulbs removed. The brains were separated into left and right hemispheres and cerebellum was removed from the left hemisphere, after which the left hemispheres were weighed and snap frozen on dry ice and stored at −80° C. until further preparation and analysis of the concentrations of injected test constructs using a Meso Scale Discovery (MSD) based assay. The right hemispheres were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing (Example 16 below).

For brain concentration measurements, frozen left hemispheres were thawed on ice and homogenized in Tris-buffered saline (TBS) containing cOmplete protease inhibitor and phosSTOP phosphatase inhibitor (#11836145001 and #04906837001, Roche) by automated bead homogenization using MP Biomedical's FastPrep-24 5G system with Lysing Matrix D for 5 s at 6 m/s. Triton X-100 (#X100, Merck) was added to the homogenate resulting in a final Triton X-100 concentration of 0.5% and a weight to volume ratio of 1:10. Homogenates were vortexed for 10 s and centrifuged at 16 000×g for 1 h at 4° C., after which supernatants were collected and used for brain antibody exposure measurements.

Brain and plasma concentrations of 2A3 #2-LC1-K322A, 2A3 #2-HC6-K322A, 2A3 #2-LC5-K322A and mAb158 hIgG1-K322A were determined using a custom MSD assay detecting human Fc. A 96-well MSD plate (#L15XA-3) was coated overnight at 4° C. with 25 ng/well goat anti-human IgG, Fcγ fragment specific antibody (#109-005-098, Jackson Immuno Research Europe Ltd) diluted in 1×PBS (#09-9400-100, Medicago AB). The coat was removed, and wells were blocked with 1% Blocker A (#R93BA-4, MSD) in PBS-0.05% Tween20 (PBS-T) (#09-9410-100, Medicago AB). Following 4× wash with 1×PBS-T, the samples and test construct calibrators diluted in 1% Blocker A in PBS-T, were added to plate and incubated in room temperature (RT) for 2 h at 900 rpm. Detection of bound antibodies was done by sequential incubations for 1 h at 900 rpm RT with the secondary antibody (mouse anti-human IgG #3850-1-1000, MT145, Mabtech), followed by 1 h at 900 rpm RT incubation with the SULFO-TAG conjugated anti-mouse detection antibody (R32AC-1, MSD). Secondary and detection antibodies were both diluted to 25 ng/well in 1% Blocker A in PBS-T. 4× wash with 1×PBS-T was performed between all incubation steps. Following the last wash, 2× Read Buffer T (#R92TC, MSD) was added prior to reading the plates in an MSD SECTOR Imager. The test construct concentration in the samples was evaluated with the MSD Discovery Workbench software, using a 4PL curve fitting algorithm and curve weighting 1/Y2 for the corresponding test construct calibrator curve.

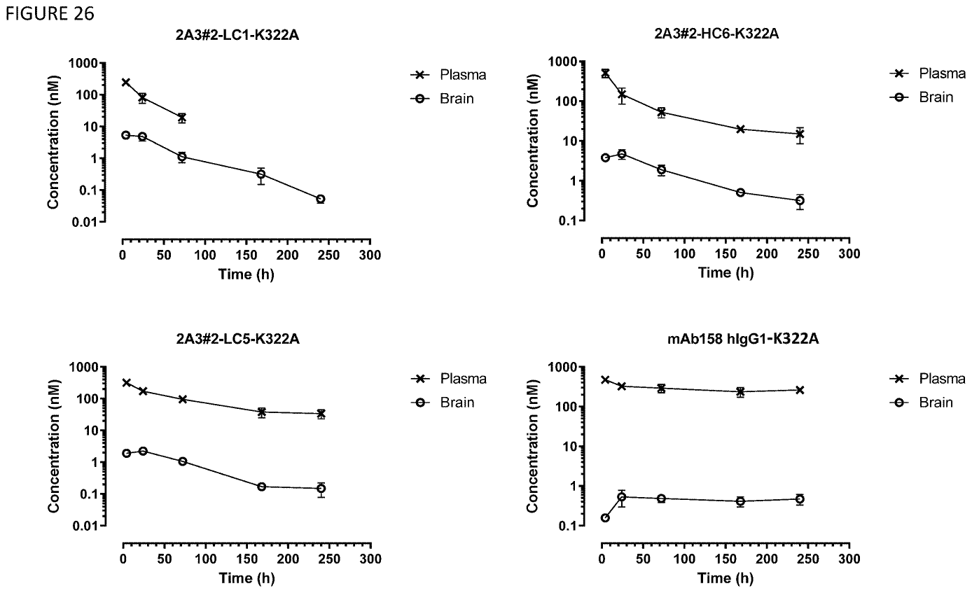

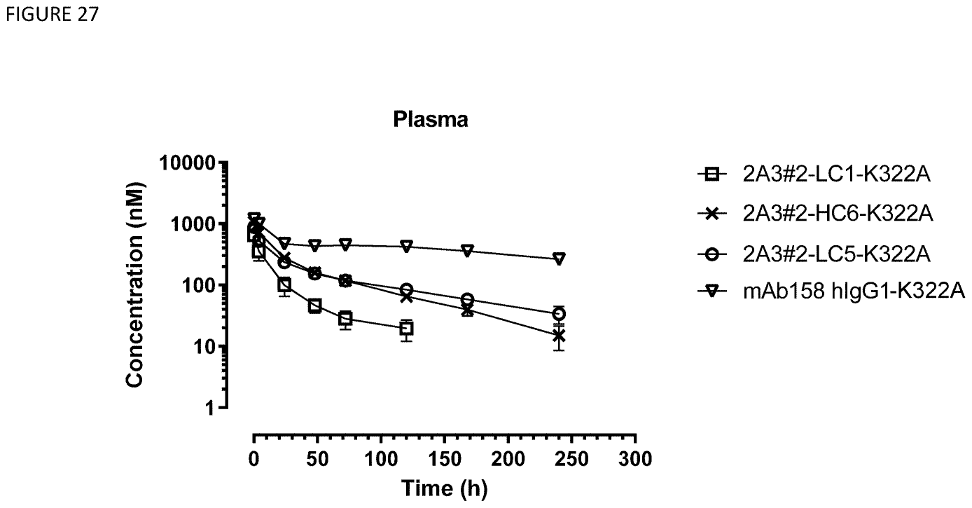

The results are shown in FIGS. 26 and 27. As shown from terminal samples in FIG. 26, a higher maximum concentration in the brain as well as higher brain exposure over time was observed for test constructs carrying a hTfR1 binding module compared to that of mAb158 hIgG1-K322A alone. As shown in FIG. 27, the plasma exposure for the test constructs carrying a hTfR1 binding module was lower compared to that of mAb158 hIgG1-K322A, indicating hTfR1 engagement and clearance of test constructs from the plasma into hTfR1 expressing tissues. Taken together, the data supports the conclusion that the test constructs undergo hTfR1-mediated BBB transport and that the affinity of the hTfR1 binding h26D3 variants influences both the brain and plasma exposure profiles.

### Example 16

### Immunohistochemistry of Gen 2A Constructs with hTfR1 Affinity Variants

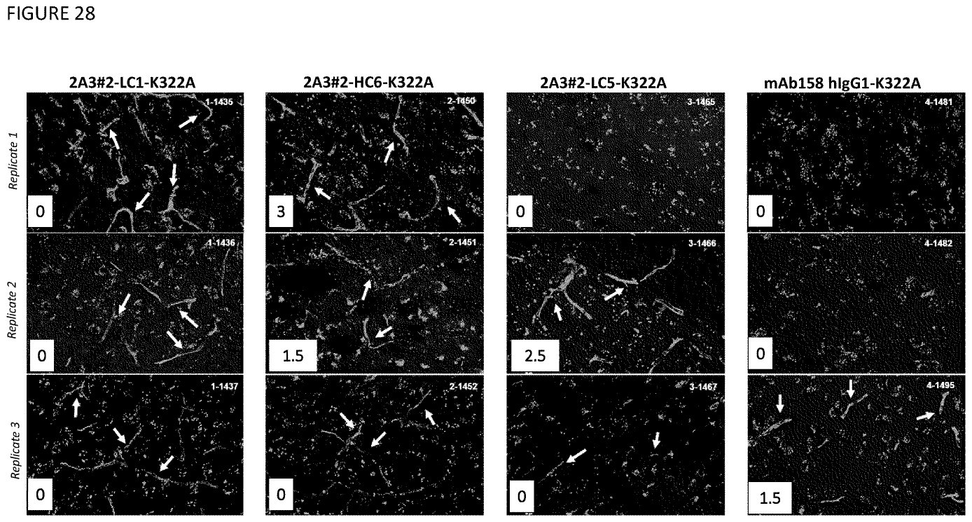

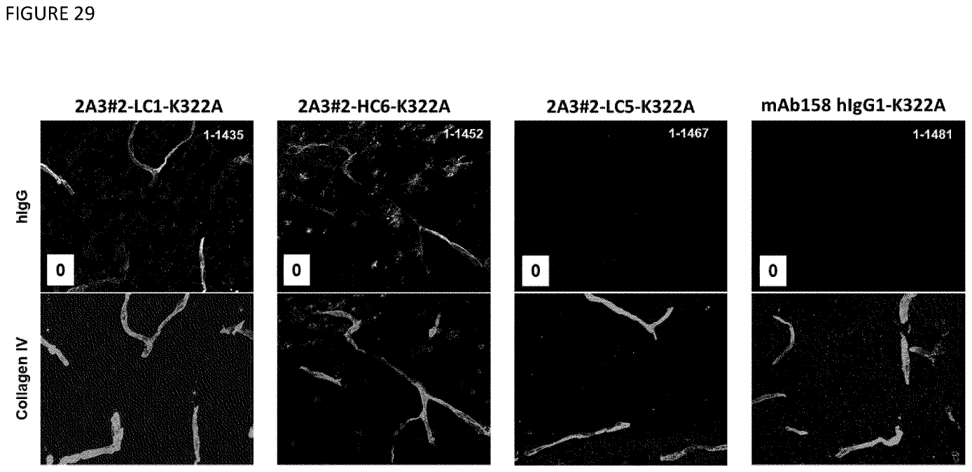

In vivo engagement of hTfR1 by the test constructs of Example 15 (2A3 #2-LC1-K322A, 2A3 #2-HC6-K322A, 2A3 #2-LC5-K322A and mAb158 hIgG1-K322A) was studied further using a qualitative immunohistochemistry (IHC) analysis. In brief, the right hemispheres in sucrose of the animals terminated in Example 15 were embedded in O.C.T. compound (LAMB/OCT, Thermo Fisher Scientific) and fast-frozen in dry ice. Embedded right hemispheres were sectioned and sagittal 20 μm slides were collected onto Superfrost cryoslides (J1800AMNZ, Thermo Fisher Scientific) and air-dried prior to IHC. The brain sections were pre-treated with M.O.M. Mouse IgG Blocking Reagent (MKB-2213-1, Vector Laboratories) for 1 h at room temperature. Primary antibodies were diluted in 1×PBS 0.1% Triton X-100 and incubated over night at 4° C., and secondary antibodies were diluted in 1×PBS and incubated for 1.5 h at room temperature. Blood vessels were visualized with anti-collagen IV (1:100) (2150-1470, Biorad) and Alexa488 anti-rabbit IgG H+L (1:500) (A21206, Invitrogen). The i.v. dosed constructs were visualized with Alexa647 anti-human IgG H+L (1:500) (A21206, Invitrogen). All incubations were conducted in a PBS humidified chamber. Slides were washed in 1×PBS in a cuvette (usually 5×5 min) after incubation. Sections were mounted with Fluoromount-G (00-4958-02, Invitrogen, USA) and images were captured using a Leica Stellaris 5 confocal system equipped with a HC PL APO 40×/1.25 GLYC motCORR CS2 (FIG. 28) and HC PL APO 63×/1.40 OIL CS2 (FIG. 29) objectives (Leica, #11506423).

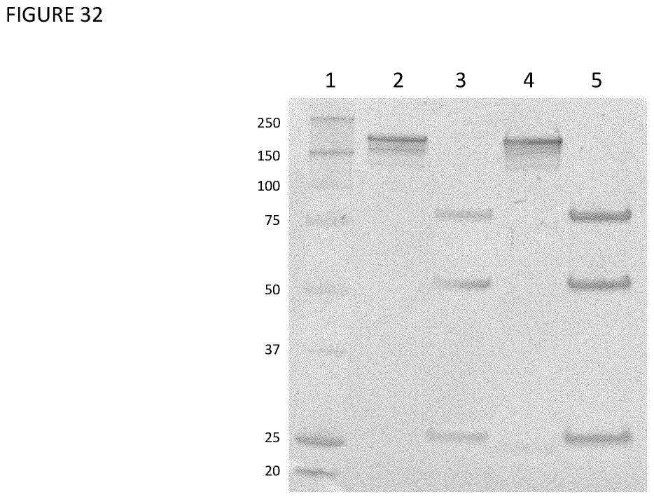

The 24 h post-dose brains showed distinct IHC immunofluorescence signals in the capillaries for the LC1 and HC6 constructs (FIG. 32). The LC5 variant and control (mAb158 hIgG1-K322A) showed no detectable immunofluorescence signal in the capillaries (FIG. 28). Because some variation between brains of the same groups was observed, a subjective score from 0 to 3 of the macroscopic perfusion success was considered when interpreting the images. The score was established based on visual examination of brains after extraction, where a score of 0 corresponded to a white brain with no visible signs of blood in any vessels, a score of 1 corresponded to a brain with slightly pink coloration and barely visible signs of blood in a few vessels, a score of 2 corresponded to a brain with pink coloration and visible signs of blood in vessels, and a score of 3 corresponded to a red brain with pronounced, visible signs of remaining blood covering most of the brain or the larger vessels. The perfusion score matches well with the images, in that those brains that had been injected with 2A3 #2-LC5-K322A and mAb158 hIgG1-K322A and exhibited an immunofluorescence signal in the capillaries were the ones that were shown to be poorly perfused. Staining with collagen IV was performed to visualize all the capillaries on the slide and to compare with dosed construct immunofluorescent signal (FIG. 29). The data indicate an active uptake of the constructs comprising hTfR1 binding variants into brain capillaries when administrated intravenously.

### Example 17

### Investigation of Infusion-Related Immune Reactions

Infusion of Fc containing biomolecules in vivo comes with a possibility of immune reactions, e.g. exhibiting such acute clinical symptoms as those described by Couch and co-workers (Couch et al (2013), Sci Transl Med 5(183):183ra57, 1-12). In order to evaluate test constructs of the disclosure with respect to such reactions, the test and control constructs given in Table 15 were designed and expressed.

TABLE 15
Test constructs and amino acid sequences

| Designation | Heavy chain | Light chain |
|---|---|---|
| 2A2#2-8D3 | SEQ ID NO: 104 | SEQ ID NO: 113 |
| 2A2#2-15G11 | SEQ ID NO: 104 | SEQ ID NO: 114 |
| 2A2#2-8D3-K322A | SEQ ID NO: 110 | SEQ ID NO: 113 |
| 2A3#10-WT | SEQ ID NO: 104 | SEQ ID NO: 115 |
| 2A3#14-WT | SEQ ID NO: 104 | SEQ ID NO: 116 |
| 2A3#3-WT | SEQ ID NO: 104 | SEQ ID NO: 117 |
| 2A3#2-WT | SEQ ID NO: 104 | SEQ ID NO: 118 |

Constructs with a murine (2A2 #2-8D3) or human (2A2 #2-15G11) high affinity, apical TfR1 binder coupled to an IgG1 with full effector function in the Fc domain were generated. 2A2 #2-8D3-K322A was generated as a complement resilient comparator to 2A2 #2-8D3. The “VL first” Gen 2A variants 2A3 #10-WT and 2A3 #14-WT and “VH first” variants 2A3 #3-WT and 2A3 #2-WT, all comprising the h26D3 TfR1 binding module of the disclosure in either “VL first” or “VH first” scFv format, were generated to investigate whether infusion reactions could be mitigated by epitope binding and binding module orientation. All test constructs were administered as single intravenous (i.v.) injections into hTfR1 knock-in (hTfR1-KI) mice which express both murine and human TfR1 (Example 6) at doses of 2.5, 11, 40 or 60 nmol/kg (n=1-3 mice per dose and test construct). Separate animals were used for each dose and each test construct. The first cohort of mice for each test construct were given the 11 nmol/kg dose.

Progression to a subsequently higher or lower dose was dependent on absence or presence of observed infusion reaction at the previous dose level, with a thorough assessment of degree and duration of observed symptoms performed in accordance with Swedish and EU animal welfare legislation and ethical approval and guidelines. The observational symptoms were assessed as mild, moderate or severe for each individual mouse. Symptoms ranged from no clinical symptoms to scruffy, hunched appearance by approximately 15-25 min post dose, isolation and inactivity, profound post-dose lethargy, slight motor dysfunction, and increased heart rate and breathing. Mild to moderate symptoms were completely reversed within hours, while severe or prolonged symptoms resulted in an immediate sacrifice of the animal. The observational outcome, with first infusion reactions (FIR) being reported as no, mild, moderate or severe reaction, is presented in Table 16.

TABLE 16
First infusion reactions after i.v. administration of test constructs

| | Dose (nmol/kg) | | | |
|---|---|---|---|---|
| Construct | 2.5 | 11 | 40 | 60 |
| 2A2#2-8D3 | | Mild to | Mild to | Moderate |
| | | moderate | moderate | |
| 2A2#2-8D3- | | None | None | |
| K322A | | | | |
| 2A2#2-15G11 | None | Severe | | |
| 2A3#10-WT | | None | None | None |
| 2A3#14-WT | | None | None | None |
| 2A3#3-WT | | None | None | None |
| 2A3#2-WT | | None | None | None to mild |

The hTfR-KI mice express both murine and human TfR1, and can therefore cross-react with both the mouse specific 8D3 and human specific 15G11-1 TfR1 binders, as well as with the h26D3 WT variants. Mild to moderate FIR was observed for 2A2 #2-8D3 up to doses of 60 nmol/kg. In line with in vitro data, the response was abolished by the introduction of the K322A mutation in the Fc domain of the cargo antibody as implemented in 2A2 #2-8D3-K322A, preventing a CDC response and thus omitting FIR dependent on complement activation. Administration of the human, apical binder 2A2 #2-15G11 resulted in severe FIR already at the 11 nmol/kg dose. No symptoms of FIR were observed after injection of 2A3 #10-WT, 2A3 #14-WT or 2A3 #3-WT up to a dose of 60 nmol/kg, while there were no or possibly mild symptoms after injection of 60 nmol/kg of 2A3 #2-WT. This supports the hypothesis that the antibody part is positioned underneath the hTfR1 binding scFv domain and thereby closer to the plasma membrane. Results indicate that the binding epitope for the scFv on hTfR1 and the binding module orientation together lead to a mitigation of observational FIR. By contrast, FIR was observed for both of the apical TfR1 binders 8D3 and 15G11-1, regardless of whether these bind to murine or human TfR1 in the hTfR1-KI mouse model.

All mice were subjected to a pre-dose blood sample and mice with reversed mild to moderate infusion reactions were also subjected to a 2 h blood sample. Both in-life blood samples were collected from the saphenous vein into Sarstedt Microvette CB300 K2E tubes for further processing to plasma and subsequent cytokine analysis. Brain and plasma exposure of test constructs were investigated at the 24 h termination timepoint.

At termination and independently of post sample analysis, animals were deeply anaesthetized using isoflurane and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with ice cold PBS. Following perfusion, brains were extracted, and the olfactory bulbs removed. The brains were separated into left and right hemispheres and cerebellum was removed from the left hemisphere, after which the left hemispheres were weighed and snap frozen on dry ice and stored at −80° C. until further preparation and analysis of the concentrations of injected test constructs, using a Meso Scale Discovery (MSD) based assay.

For brain concentration measurements, frozen left hemispheres were thawed on ice and homogenized in Tris-buffered saline (TBS) containing cOmplete protease inhibitor and phosSTOP phosphatase inhibitor (#11836145001 and #04906837001, Roche) by automated bead homogenization using MP Biomedical's FastPrep-24 5G system with Lysing Matrix D for 5 s at 6 m/s. Triton X-100 (#X100, Merck) was added to the homogenate resulting in a final Triton X-100 concentration of 0.5% and a weight to volume ratio of 1:10. Homogenates were vortexed for 10 s and centrifuged at 16 000×g for 1 h at 4° C., after which supernatants were collected and used for brain antibody exposure measurements

Brain and plasma concentrations of test constructs 2A3 #2-WT, 2A3 #3-WT, 2A3 #10-WT, 2A3 #14-WT and 2A2 #2-8D3 were determined using a custom MSD assay detecting the human Fc. A 96-well MSD plate (#L15XA-3) was coated overnight at 4° C. with 25 ng/well goat anti-human IgG, Fcγ fragment specific antibody (#109-005-098, Jackson Immuno Research Europe Ltd) diluted in 1×PBS (#09-9400-100, Medicago AB). The coat was removed, and wells were blocked with 1% Blocker A (#R93BA-4, MSD) in PBS-0.05% Tween20 (PBS-T) (#09-9410-100, Medicago AB). Following 4× wash with 1×PBS-T, the samples and test construct calibrators diluted in 1% Blocker A in PBS-T, were added to plate and incubated in room temperature (RT) for 2 h at 900 rpm. Detection of bound antibodies was done by sequential incubations for 1 h at 900 rpm RT with the secondary antibody (mouse anti-human IgG #3850-1-1000, MT145, Mabtech), followed by 1 h at 900 rpm RT incubation with the SULFO-TAG conjugated anti-mouse detection antibody (R32AC-1, MSD). Secondary and detection antibodies were both diluted to 25 ng/well in 1% Blocker A in PBS-T and 4× wash with 1×PBS-T was performed between all incubation steps. Following the last wash, 2× Read Buffer T (#R92TC, MSD) was added prior reading the plates in an MSD SECTOR Imager. The test construct concentration in the samples was evaluated with the MSD Discovery Workbench software, using a 4PL curve fitting algorithm and curve weighting 1/Y2 for the corresponding test construct calibrator curve. For the construct 2A2 #2-8D3, 2A3 #2-WT was used as calibrator.

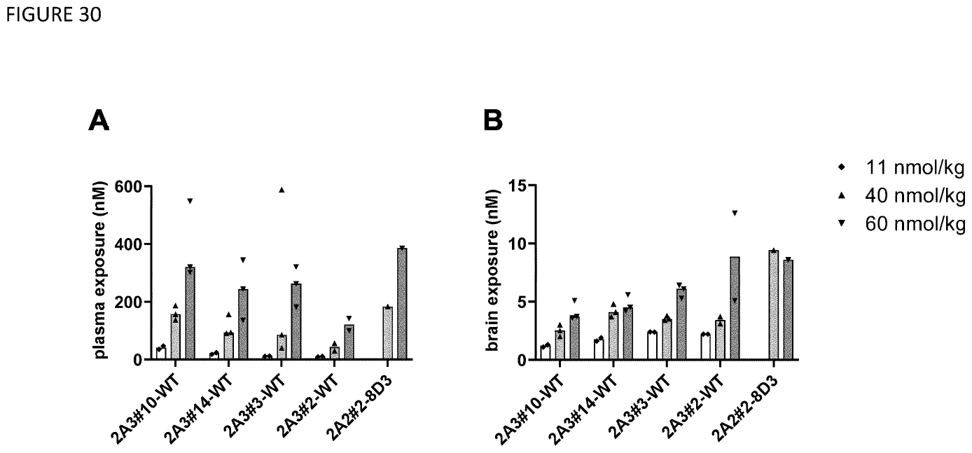

Brain and plasma concentrations at 24 h post dose of indicated test constructs are shown in FIG. 30. As shown in FIG. 30, dose increases were reflected in increased plasma and brain exposure, demonstrating successful administration of the test items and engagement with the TfR1 in vivo.

Plasma concentrations of ten different cytokines (IFNγ, IL-1β, IL-2, IL-4, IL-5, IL-6, IL-10, IL-12p70, KC/GRO and TNF) were determined using the pre-made panel V-PLEX Plus Proinflammatory Panel 1 Mouse Kit (K15048G, Meso Scale Discovery (MSD)) following the manufacturer's instruction. Briefly, the plates were incubated with calibrator, control samples and plasma samples diluted 10× for 2 h, after which a mixture of all 10 SULFO-TAG detection antibodies were added for 2 h. All incubations were performed at 900 rpm at room temperature. Before and after each incubation step, a 4× wash in PBS-0.05% Tween20 (PBS-T) (#09-9410-100, Medicago AB) was performed. After the last wash, 2× Read Buffer T (#R92TC, MSD) was added before reading the plates in an MSD SECTOR Imager. The concentration of the analytes in the samples was evaluated with the MSD Discovery Workbench software, using a 4PL curve fitting algorithm and curve weighting 1/Y2 for the corresponding calibrator standard curve.

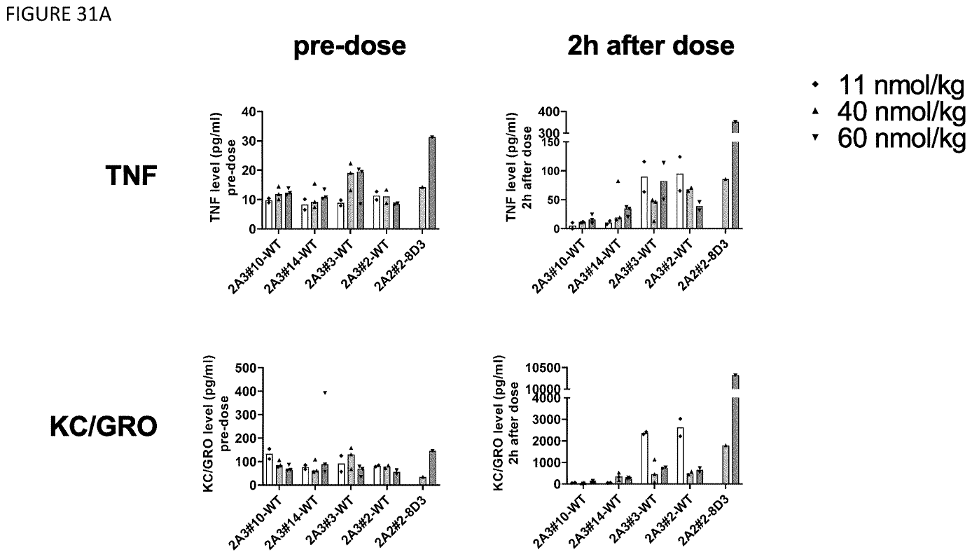

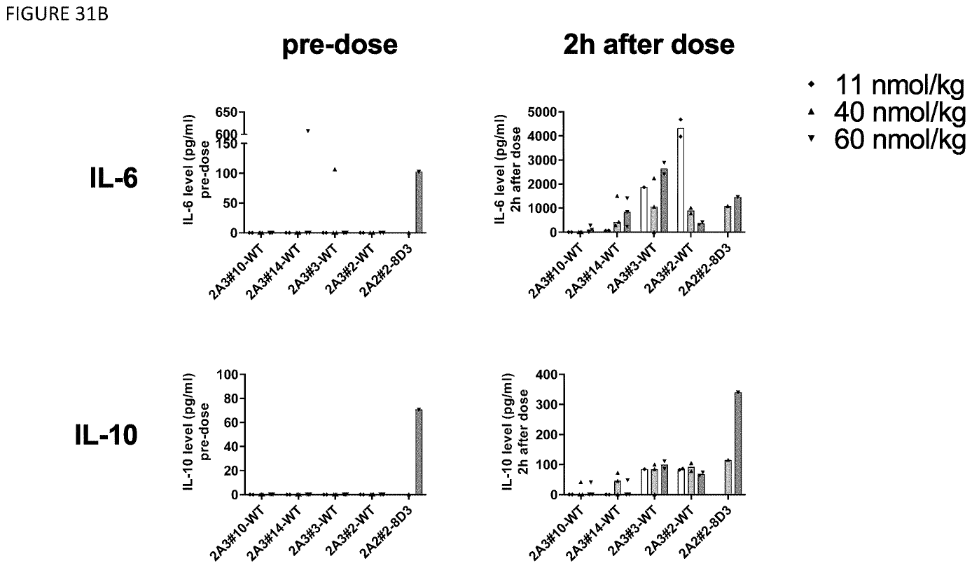

The results for a subset of relevant tested cytokines are shown in FIG. 31. The tested constructs elicited different cytokine responses, with constructs 2A3 #3-WT and 2A3 #2-WT (“VH first”) responding more strongly than constructs 2A3 #10-WT and 2A3 #14-WT (“VL first”). The results demonstrate that positioning the antibody moiety under the h26D3 binder moiety and closer to the plasma membrane induces lower levels of cytokines and chemokines. Construct 2A2 #2-8D3, which binds to TfR1 on the apical domain and exhibited FIR based on the observation data above (Table 16), generated high levels of cytokines/chemokines, especially of KC/GRO and IL-10.

### Example 18

### Production and Purification of Designed Gen 2D Constructs

Two bispecific constructs designed as knob-into-hole antibody variants were expressed, each containing an scFv of h26D3 linked to the C-terminal amino acid residue of the knob heavy chain of the antibody. In the first variant, “mAb158-Gen2D-h26D3 VH first” (SEQ ID NO:105), the N-terminal amino acid residue of the VH region of the scFv was linked to the Fc. In the second variant, “mAb158-Gen2D-h26D3 VL first” (SEQ ID NO:106), the N-terminal amino acid residue of the VL region of the scFv was linked to the Fc. The hole heavy chain used in both constructs is represented by SEQ ID NO:107, whereas the light chain present in two copies in each antibody part is represented by SEQ ID NO:108.

The designed “Gen 2D” constructs were expressed by transient transfection of Chinese hamster ovary cells (ExpiCHO; Thermo Fisher Scientific) according to the manufacturer's instructions. Filtered cell culture supernatants were applied to a MabSelect SuRe column (Cytiva) which was subsequently washed with DPBS pH 7.4. Expressed binding proteins were eluted by application of 0.7% HAc pH 2.5, followed by neutralization of the sample to pH 7.5. Purified samples were polished further by either size exclusion chromatography (SEC; HiLoad 26/600 Superdex 200; Cytiva) in DPBS pH 7.4 or anion exchange with for example with a HiTrap Q HP column (Cytiva) and 20 mM Trizma as binding buffer and elution with NaCl. Each purified expressed construct was characterized using SDS-PAGE, size-exclusion chromatography (Superdex 200 Increase 3.2/300; Cytiva) and UV protein concentration determination. An example of the purity of the different constructs is shown in FIG. 32 from a representative SDS-PAGE analysis. The non-reduced gel showed one band around 175 kDa, whereas the reduced gel shows the expected three bands from the Gen 2D format: the “knob” heavy chain fused to the h26D3 scFv showing at around 75 kDa, the identical light chains at approximately 25 kDa and the “hole” heavy chain without fused scFv at approximately 50 kDa.

### Example 19

### Analysis of Gen 2D Bispecific Binding Proteins to hTfR1 Using SPR

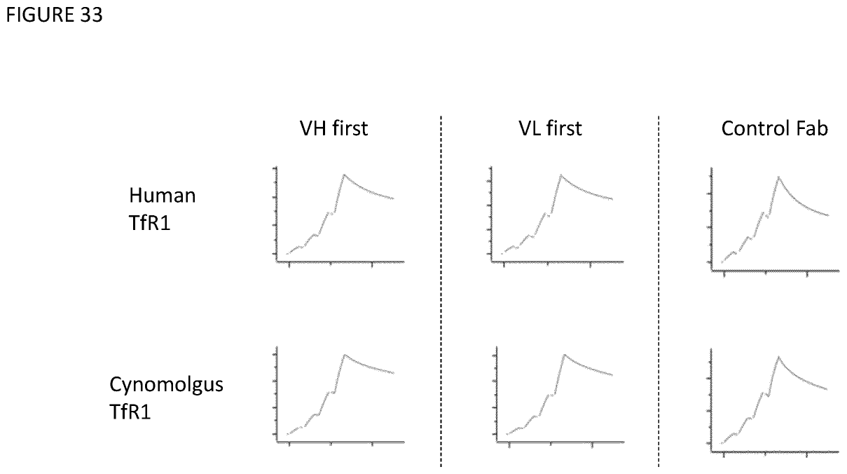

Binding of the purified bispecific binding proteins to hTfR1 and cTfR1 was evaluated as described for the Gen 2A constructs in Example 12. The data in FIG. 33 shows that all designed and produced Gen 2D constructs bind to hTfR1. All constructs show similar on-rates and off-rates against hTfR1 (SEQ ID NO:86) and cTfR1 (SEQ ID NO:87) as compared to a control Fab fragment of the hTfR1 binder h26D3. The results illustrate that all produced constructs are functional and that the “VH first”/“VL first” configuration does not influence the binding of the constructs to hTfR1.

### Example 20

### ADCC Measurements

To investigate the effector function of the Gen 2D constructs, an antibody-dependent cellular cytotoxicity (ADCC) assay was used. In order to evaluate the ADCC activity, Jurkat effector cells (Promega; #G7018) were used. The cells stably express FcγRIIIa receptor, V158 (high affinity) variant, and an NFAT response element driving expression of firefly luciferase as a measure of ADCC activity. When an Fc part is bound to FcγR, the activation signal for the effector cells is triggered leading to killing of those target cells that are coated with antibodies on their surface. Ramos cells (Sigma, cat: 85030802), which express high levels of hTfR1 on the cell surface, were used as target cells. Effector and target cells were used in a ratio of effector:target of 6:1, with and without serially diluted test construct. Controls used were antibody alone, i.e. without hTfR1 binding scFv, as negative control and rituximab as positive control. Target cells with test constructs were plated in a 96 well assay plate (Corning, #3917), mixed with effector cells and incubated for 6 h at 37° C. with 5% CO₂. When an Fc-containing protein forms a bridge between target and effectors (through the interaction between Fc and FcγR), it leads to luciferase activity. After 6 h incubation, Bio-Glo luciferase reagent was added and the luciferase signal quantitated in a SPARK plate reader (Tecan).

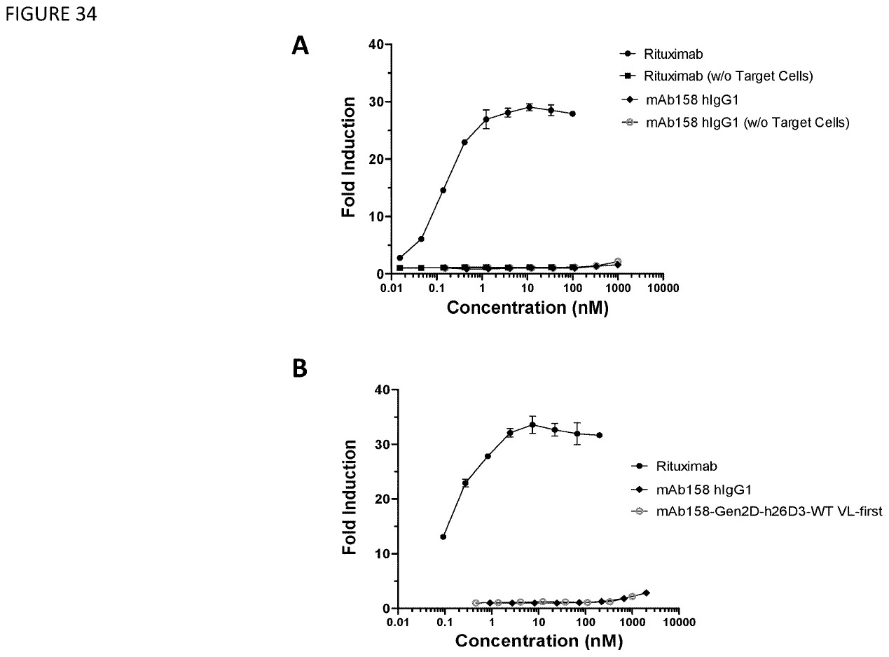

First, the antibody rituximab was used as a positive control to verify ADCC activity and fold induction (FIG. 34A). Rituximab is known to be a strong inducer of ADCC, and this was verified in the assay setup. When the target cell is excluded, there is no induction of ADCC. Importantly, the antibody used in the Gen 2D tested constructs (mAb158) possesses no ADCC activity when tested on its own which is also shown, as there is no ADCC activity for mAb158 when target cells are excluded (FIG. 34A). Next, the Gen 2D construct “mAb158-Gen2D-h26D3 VL first”, expressed and analyzed as described in Examples 18-19, was investigated. Importantly, no ADCC activity was detected for this construct (FIG. 34B) even though it binds strongly to the target cell via the hTfR1 binder.

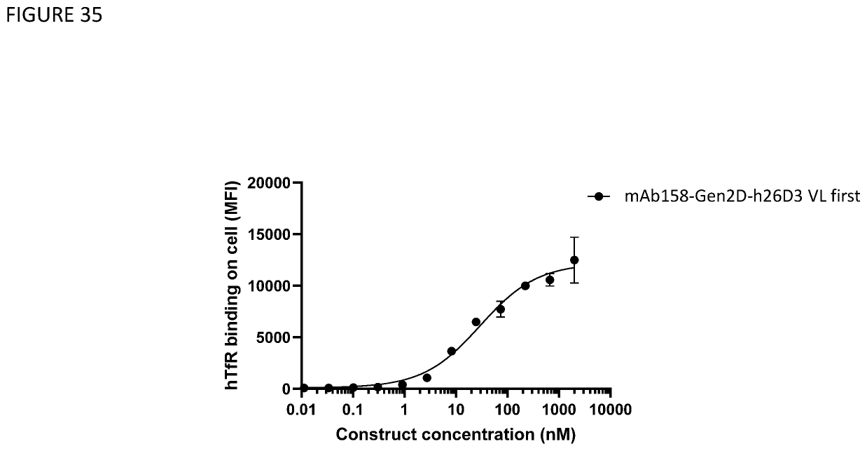

Next, K562 cells (Sigma/ECACC) were used in a cell binding experiment. Fcγ receptors were blocked using Fc receptor blocker (Innovex biosciences, #NB309-4X-40) for 30 min at 4° C., after which the cells were washed in PBS. The cells were seeded in a 96 well V-bottom plate (#249570, Thermo Scientific Nunc) and a serial dilution of the tested Gen 2D construct “mAb158-Gen2D-h26D3 VL first” was added. The plate was incubated at 4° C. overnight. The cells were washed with PBS containing 1% BSA and then fixed with freshly prepared 4% formaldehyde (Thermo Scientific™ Pierce™, #28906) diluted in PBS for 10 min at room temperature. The cells were washed with PBS containing 1% BSA, and then stained with fluorescently labelled secondary goat F(ab′)₂ anti-human IgG(γ)-Alexa fluor 488 (Invitrogen Life technologies, #H10120). Staining was carried out for 30 min at 4° C. After incubation with the detection reagent, cells were washed with PBS, 1% BSA. The cells were finally resuspended in 200 μl PBS with 1% BSA and acquired using a BD FACSLyric flow cytometer system (BD Biosciences). Samples were analyzed using flowJo software (BD Biosciences). The measured median fluorescence intensities (MFI) were plotted against binding protein concentration and displayed in FIG. 35. The results show that the tested Gen 2D construct binds to hTfR1 on the surface of the cells.

Taken together, the ADCC and cell binding experiments indicate that in the Gen 2D construct with the h26D3 scFv in the “VL first” configuration, the Fc part present in the binding protein cannot engage with Fcγ receptors and lead to ADCC, despite being bound to the cell surface via hTfR1.

### Example 21

### Plasma and Brain Exposure of Gen 2D Construct In Vivo

To further evaluate the h26D3 HC6 binding module as an scFv moiety in the Gen 2D format together with an antibody binding amyloid P, brain and plasma exposure of such a test construct was investigated over time in hTfR1 knock-in (hTfR1-KI) mice (Example 6). In vivo target engagement to brain amyloid 3 pathology was investigated at 72 h post-dose in crossed 5×FAD×hTfR-KI mice. The 5×FAD×hTfR-KI mice were generated by crossing 5×FAD male mice on a C57BL/6J background (Northwestern University) with hTfR-KI females. The 5×FAD mouse model is an Alzheimer's disease (AD) model with mice expressing human APP and PSEN1 transgenes with a total of five AD-linked mutations, including the Swedish (K670N/M671L), Florida (I716V), and London (V717I) mutations in APP, and the M146L and L286V mutations in PSEN1.

In the constructs tested in this example, the amyloid P binding antibody used in previous examples, mAb158, was replaced by another amyloid P binding antibody denoted mAb000 herein. The construct was created on the same format as “mAb158-Gen2D-h26D3 VH first” of Example 18 and denoted mAb000-Gen2D-h26D3-HC6. To examine exposure of the test construct and the control antibody in brain and plasma, mAb000-Gen2D-h26D3-HC6 and mAb000 were generated and injected intravenously (i.v.) into hTfR1-KI mice (n=11 per test construct) at equimolar doses of 40 nmol/kg (corresponding to approximately 6-7 mg/kg). Terminal plasma and brain exposure were assessed at three consecutive termination timepoints of 24, 72 and 336 h, respectively, with n=3-5 mice per timepoint and test construct. Blood for the assessment of continuous plasma concentration versus time profiles were sampled from animals sacrificed at the 336 h timepoint (n=5), and at the following timepoints 0.25, 4, 24, 48, 72, 168, 240 and 336 h after test construct administration. In-life blood samples were sampled from the saphenous vein into Sarstedt Microvette CB300 K2E tubes.

To investigate brain amyloid 3 target engagement, mAb000 and mAb000-Gen2D-h26D3-HC6 were injected intravenously (i.v.) into 5×FAD×hTfR-KI mice (n=2-3 per test item) at equimolar doses of 40 nmol/kg (corresponding to approximately 6-7 mg/kg). The animals were then terminated at 72 h post dose.

At the individual termination timepoints, independent of post sample analysis, animals were deeply anaesthetized using isoflurane and terminal blood samples were collected from the orbital plexus into BD Microtainer K2EDTA tubes. The samples were inverted and centrifuged at 2400×g for 10 min at 4° C. Plasma was extracted and transferred to Eppendorf tubes and frozen at −80° C. Immediately following blood sampling, the abdomen of the animals was cut open and a cannula (21 G) was inserted into the left ventricle of the heart. A small cut was made in the right atrium and transcardial perfusion was performed with ice cold PBS. Following perfusion, brains were extracted, and the olfactory bulbs removed. The brains were separated into left and right hemispheres and cerebellum was removed from the left hemisphere, after which the left hemispheres were weighed and snap frozen on dry ice and stored at −80° C. until further preparation and analysis of the concentrations of injected test constructs, using a Meso Scale Discovery (MSD) based assay. The right hemispheres were placed in 4% formaldehyde and stored at 4° C. for 24 h, after which they were rinsed in cold PBS, transferred to cold 30% sucrose solution prepared in PBS and stored at 4° C. for further immunohistochemistry (IHC) processing (see Example 22 below).

For brain concentration measurements, frozen left hemispheres were thawed on ice and homogenized in Tris-buffered saline (TBS) containing cOmplete protease inhibitor and phosSTOP phosphatase inhibitor (#11836145001 and #04906837001, Roche) by automated bead homogenization using MP Biomedical's FastPrep-24 5G system with Lysing Matrix D for 5 s at 6 m/s. Triton X-100 (#X100, Merck) was added to the homogenate resulting in a final Triton X-100 concentration of 0.5% and a weight to volume ratio of 1:10. Homogenates were vortexed for 10 s and centrifuged at 16 000×g for 1 h at 4° C., after which supernatants were collected and used for brain antibody exposure measurements.

Brain and plasma concentrations of mAb000 and mAb000-Gen2D-h26D3-HC6 were determined using a custom MSD assay detecting the human Fc. A 96-well MSD plate (#L15XA-3) was coated overnight at 4° C. with 25 ng/well goat anti-human IgG, Fcγ fragment specific antibody (#109-005-098, Jackson Immuno Research Europe Ltd) diluted in 1×PBS (#09-9400-100, Medicago AB). The coat was removed, and wells were blocked with 1% Blocker A (#R93BA-4, MSD) in PBS-0.05% Tween20 (PBS-T) (#09-9410-100, Medicago AB). Following 4× wash with 1×PBS-T, the samples and test construct calibrators diluted in 1% Blocker A in PBS-T were added to plate and incubated in room temperature (RT) for 2 h at 900 rpm. Detection of bound antibodies was done by sequential incubations for 1 h at 900 rpm RT with the secondary antibody (mouse anti-human IgG #3850-1-1000, MT145, Mabtech), followed by 1 h at 900 rpm RT incubation with the SULFO-TAG conjugated anti-mouse detection antibody (R32AC-1, MSD). Secondary and detection antibodies were both diluted to 25 ng/well in 1% Blocker A in PBS-T and 4× wash with 1×PBS-T was performed between all incubation steps. Following the last wash, 2× Read Buffer T (#R92TC, MSD) was added prior reading the plates in an MSD SECTOR Imager. The test construct concentration in the samples was evaluated with the MSD Discovery Workbench software, using a 4PL curve fitting algorithm and curve weighting 1/Y2 for the corresponding test construct calibrator curve.

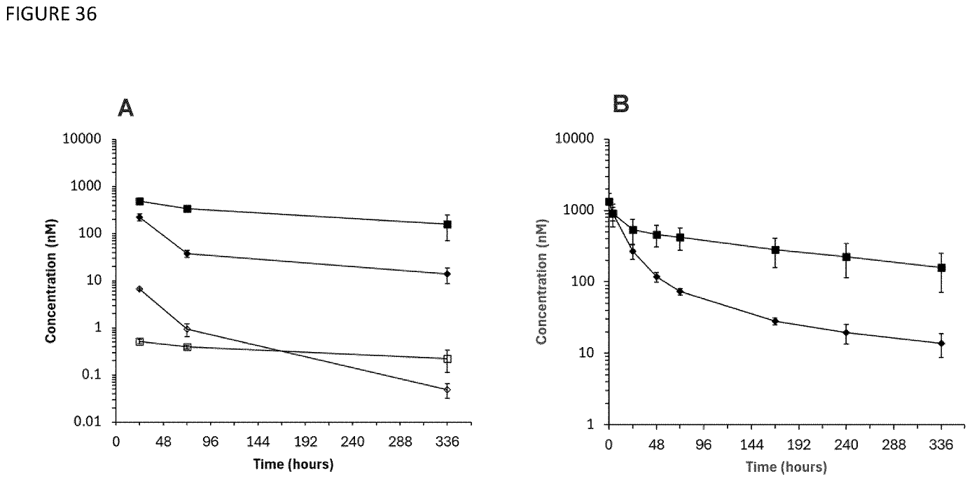

The results are shown in FIG. 36. As shown in FIG. 36A, a higher maximum concentration in brain as well as higher brain exposure, taken as the area under the curve, were observed over the time period studied for mAb000-Gen2D-h26D3-HC6 as compared to mAb000. As shown in FIG. 36B, the plasma exposure of mAb000-Gen2D-h26D3-HC6 was lower compared to mAb000, indicating hTfR1 engagement and clearance of test constructs from the plasma to hTfR1 expressing tissues. Taken together, the data supports the conclusion that the test construct mAb000-Gen2D-h26D3-HC6 undergoes hTfR1-mediated BBB transport.

### Example 22

### Immunohistochemistry of Gen 2D Construct In Vivo

In vivo engagement of both hTfR1 and amyloid P by the test construct mAb000-Gen2D-h26D3-HC6 and control antibody mAb000 (see Example 21) was studied further using a qualitative immunohistochemistry (IHC) analysis. In brief, the right hemispheres in sucrose of the animals terminated in Example 21 were embedded in O.C.T. compound (LAMB/OCT, Thermo Fisher Scientific, USA) and fast-frozen in dry ice. Embedded right hemispheres were sectioned and sagittal 20 μm slides were collected onto Superfrost cryoslides (J1800AMNZ, Thermo Fisher Scientific) and air-dried prior to IHC. The brain sections were pre-treated for 20 min with 4% PFA (HL96753.1000, HistoLab, Sweden) followed by 5 min wash with dH₂O and 5 min incubation with 70% FA for antigen retrieval. After washing 2×10 min with 1×PBS, the slides were blocked with M.O.M. Mouse IgG Blocking Reagent (MKB-2213-1, Vector Laboratories) for 1 h at room temperature. Primary antibodies were diluted in 1×PBS 0.1% Triton X-100 and incubated over night at 4° C., and secondary antibodies were diluted in 1×PBS and incubated for 1.5 h at room temperature.

Amyloid 3 was visualized with murine antibodies 6E10 (1 μg/ml) (803002, Biolegend) and 4G8 (1 ag/ml) (800702, Biolegend) and Alexa555 anti-mouse IgG H+L (1:500) (A21424, Invitrogen). The tested compounds were visualized with Alexa647 anti-human IgG H+L (1:500) (A21206, Invitrogen). All incubations were conducted in a PBS humidified chamber. Slides were washed in 1×PBS in a cuvette (usually 5×5 min) after incubation. Sections were mounted with Fluoromount-G (00-4958-02, Invitrogen) and images were captured using a Leica Stellaris 5 confocal system equipped with a HC PL APO 40×/1.25 GLYC motCORR CS2 objective (Leica, #11506423).

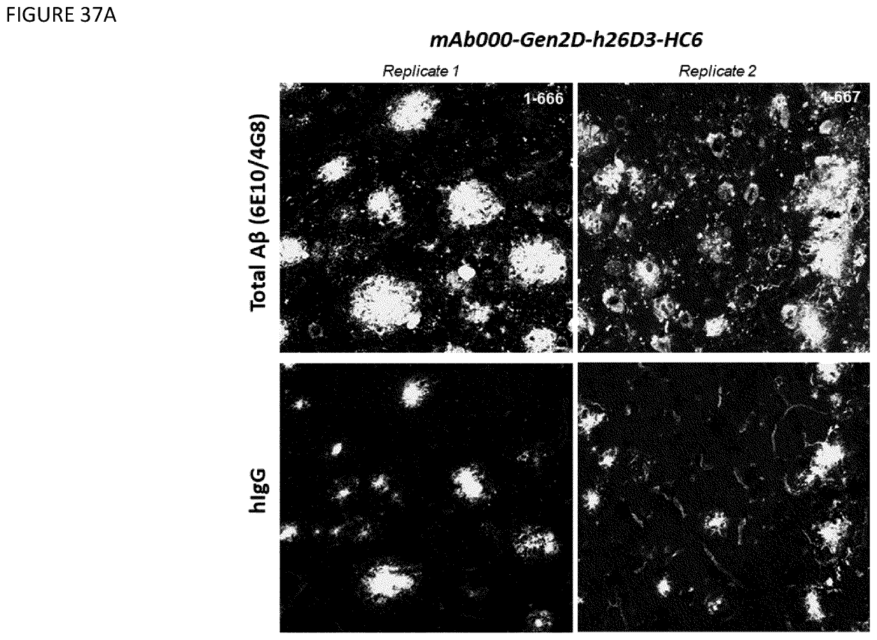

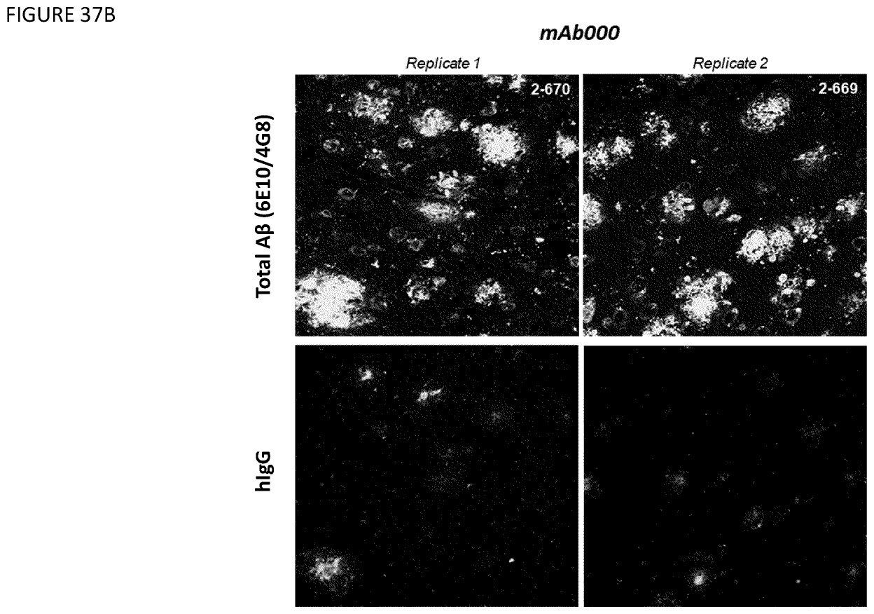

The resulting images are shown in FIG. 37. Co-localization of hIgG and amyloid P antibodies was observed, illustrating extensive amyloid P target engagement for mAb000-Gen2D-h26D3-HC6 in 7 months old 5×FAD/hTfR-KI brain at 72 h post-dose (FIG. 37A). Core plaques were predominantly positive for mAb000-Gen2D-h26D3-HC6 while a low amyloid 3 plaque co-localization was seen for the mAb000 antibody lacking hTfR1 binding module (FIG. 37B).

### Example 23

### Competition of Gen 2D Constructs for hTfR1 Binding with Antibody M-A712

In a complementary experiment to the study described in Example 3, another method was utilized to study competition of test constructs for hTfR1 binding. The test constructs investigated were “mAb158-Gen2D-h26D3 VH first” and “mAb158-Gen2D-h26D3 VL first” of Example 18 and “mAb000-Gen2D-h26D3-HC6” of Example 21, in both “VH first” and “VL first” configurations, here denoted “mAb000-Gen2D-h26D3-HC6 VH first” and “mAb000-Gen2D-h26D3-HC6 VL first”, respectively.

The method of this example uses the anti-CD71 (anti-hTfR1) antibody M-A712 as a marker for a specific epitope on the apical domain of hTfR1. The M-A712 antibody is reported to bind hTfR1 residues 208-212 in the apical domain (Radoshitzky et al (2008), PNAS 105(7):2664-2669; Maier et al (2016), Molecular Therapy Nucleic Acids 5:e321). This site overlaps with the described binding site on human TfR1 for human ferritin (Montemiglio et al (2019), Nat Commun. 10(1):1121). The binding to hTfR1 of the binders according to the disclosure, i.e. binding to the protease-like domain of hTfR1 and identified as described in Example 1, was evaluated in competition with the M-A712 antibody. In addition, M-A712 was also investigated in competition with recombinant human ferritin heavy chain 1(FTH1).

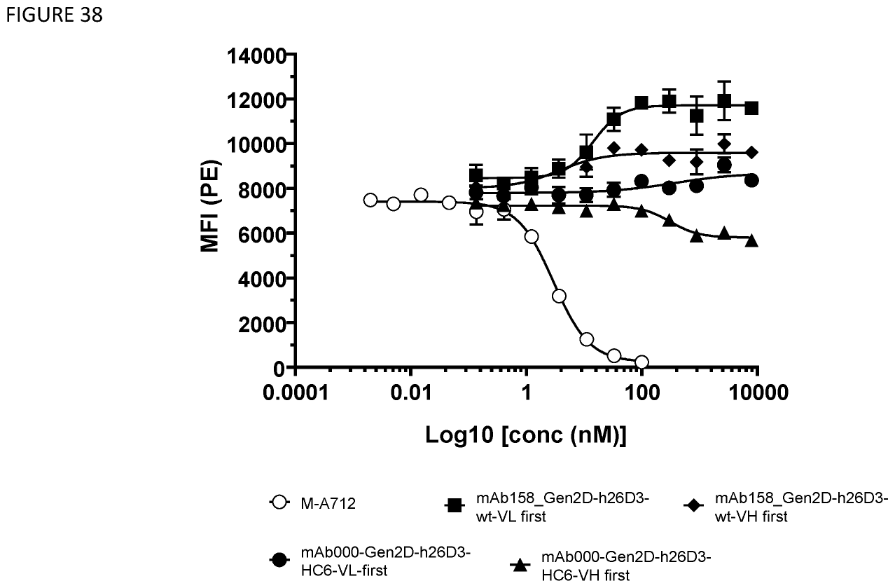

For the competition experiments, K562 lymphoblast cells (Sigma) were used. For evaluating the competition between M-A712 and the disclosed binders and confirm binding of the labeled M-A712 antibody, cells were first incubated with human Fc block (BD Pharmingen, 564220) for 30 min at 4° C., to block non-specific Fc receptor-mediated antibody binding. The cells were subsequently incubated with serially diluted test constructs along with PE conjugated M-A712 antibody (monoclonal, BD Pharmingen, 555537) and incubated for 1 h at 4° C. After the incubation, the cells were washed 3 times in staining buffer (1% BSA, 0.1% sodium azide in 1×DPBS). The M-A712 antibody as bound to hTfR1 on cell surfaces was analyzed using flow cytometry, and the mean fluorescence intensity (MFI (PE)) was plotted. FIG. 38 shows that there is no direct competition between the h26D3 binder in the different indicated constructs and M-A712. When non-labeled (unconjugated) M-A712 antibody was used as positive control for competition, the binding of labeled (PE) M-A712 signal was reduced in a concentration dependent way. The experiment illustrates that a binder directed against the protease-like domain of TfR1 does not compete directly for the same epitope as the M-A712 antibody.

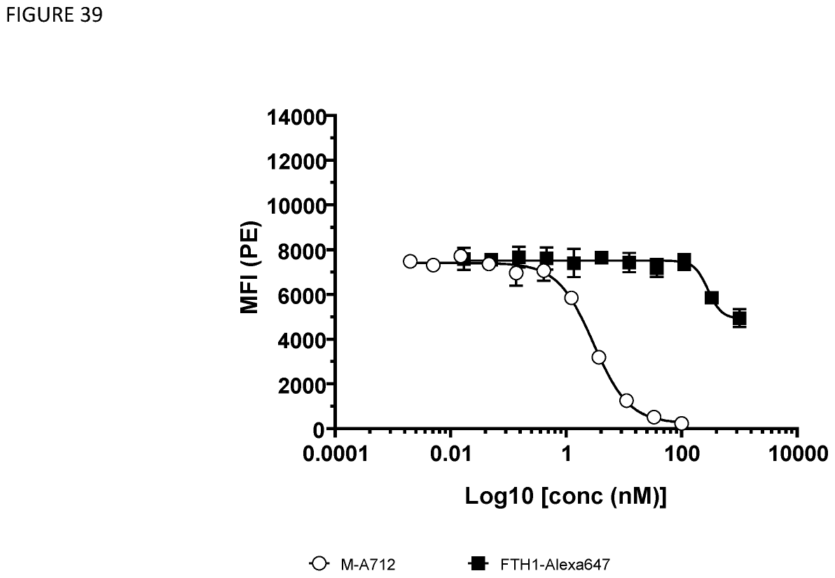

A similar experiment was conducted with human ferritin heavy chain 1 (FTH1; Sino Biologicals #13217-HNAE, Lot LC15NO0415) labeled with Alexa647. As seen in FIG. 39, competition was demonstrated at a higher concentration compared to the positive control for competition (M-A712). This data clearly demonstrates that hTfR1 binders according to the disclosure, binding to the protease-like domain of hTfR1, do not compete with the described epitope on the apical domain that corresponds to the binding site used by the ferritin protein.

### ITEMIZED LISTING OF EMBODIMENTS

1. A transferrin receptor 1 (TfR1) binding molecule, capable of selective binding to an epitope located in the protease-like domain of TfR1 defined by amino acid residues 121-183 and 384-605 in SEQ ID NO:85.

2. Binding molecule according to item 1, said epitope comprising or consisting of the amino acid residues 150, 151, 154, 158, 159, 161, 163 and 385 in SEQ ID NO:85.

3. Binding molecule according to any preceding item, comprising an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope.

4. Binding molecule according to item 3, in which said antigen-binding surface is composed of three complementarity-determining regions (CDRs) from said VH region and three CDRs from said VL region, and in which said CDRs comprise the following:

VHCDR1:
(SEQ ID NO: 1)
X1X2NMX3,

wherein
X1 is selected from D and A;
X2 is selected from Y and A; and
X3 is selected from D and A;

VHCDR2:
(SEQ ID NO: 2)
X4INPX5X6X7TTSX8X9X10KFKG,

wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;

VLCDR1:
(SEQ ID NO: 4)
KSSQSLLX11SX12NX13KNX14LA,

wherein
X11 is selected from Y and A;
X12 is selected from T and S;
X13 is selected from Q and R; and
X14 is selected from Y and A;

VLCDR2:
(SEQ ID NO: 5)
X15ASTRES

wherein
X15 is selected from W and A; and

VLCDR3:
(SEQ ID NO: 6)
QQX16X17X18X19PX20T

wherein
X16 is selected from Y and A;
X17 is selected from F and Y;
X18 is selected from I and N;
X19 is selected from Y and A; and
X20 is selected from R and Y.

5. Binding molecule according to item 4, said CDRs further comprising

VHCDR3:
(SEQ ID NO: 3)
GGX21SGSSX22X23HPMX24X25

wherein
X21 is selected from Y and A;
X22 is selected from Y and A;
X23 is selected from Y and A;
X24 is selected from D and A; and
X25 is selected from Y and A.

6. Binding molecule according to any one of items 4-5, in which said VHCDR2 is:

VHCDR2:
(SEQ ID NO: 7)
X4INPX5X6X7TTSX8NEKFKG,

wherein
X4 is selected from D and A;
X5 is selected from D and A;
X6 is selected from Y and A;
X7 is selected from D and A; and
X8 is selected from Y and A.

7. Binding molecule according to any one of items 4-6, in which said VLCDR1 is:

VLCDR1:
(SEQ ID NO: 8)
KSSQSLLX11STNQKNX14LA,

wherein
X11 is selected from Y and A; and
X14 is selected from Y and A.

8. Binding molecule according to an one of items 4-7 in which said VLCDR3 is:

VLCDR3:
(SEQ ID NO: 9)
QQX16FIX19PRT

wherein
X16 is selected from Y and A;
X19 is selected from Y and A.

9. Binding molecule according to any one of items 4-8, in which the amino acid sequence of said VHCDR1 is selected from the group consisting of SEQ ID NO:10 and 16-18.

10. Binding molecule according to any one of items 4-9, in which the amino acid sequence of said VHCDR2 is selected from the group consisting of SEQ ID NO:11, 19-23 and 34, for example selected from the group consisting of SEQ ID NO:11 and 19-23.

11. Binding molecule according to any one of items 4-10, in which the amino acid sequence of said VHCDR3 is selected from the group consisting of SEQ ID NO:12, 24-28 and 35, for example selected from the group consisting of SEQ ID NO:12 and 24-28.

12. Binding molecule according to any one of items 4-11, in which the amino acid sequence of said VLCDR1 is selected from the group consisting of SEQ ID NO:13, 29, 30 and 36, for example selected from the group consisting of SEQ ID NO:13, 29 and 30.

13. Binding molecule according to any one of items 4-12, in which the amino acid sequence of said VLCDR2 is selected from the group consisting of SEQ ID NO:14 and 31.

14. Binding molecule according to any one of items 4-13, in which the amino acid sequence of said VLCDR3 is selected from the group consisting of SEQ ID NO:15, 32, 33 and 37, for example selected from the group consisting of SEQ ID NO:15, 32 and 33.

15. Binding molecule according to any one of items 4-14, in which the amino acid sequences of the six CDRs are the following:

VHCDR1:
(SEQ ID NO: 10)
DYNMD,
VHCDR2:
(SEQ ID NO: 11)
DINPDYDTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

16. Binding molecule according to any one of items 4-14, in which the amino acid sequences of the six CDRs are the following:

VHCDR1:
(SEQ ID NO: 10)
DYNMD,
VHCDR2:
(SEQ ID NO: 21)
DINPDADTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

17. Binding molecule according to item 4, in which the amino acid sequences of the six CDRs are the following:

VHCDR1:
(SEQ ID NO: 10)
DYNMD,
VHCDR2:
(SEQ ID NO: 34)
DINPNYDTTSYSQKFKG,
VHCDR3:
(SEQ ID NO: 35)
SEAGNYYWYFDV
VLCDR1:
(SEQ ID NO: 36)
KSSQSLLYSSNRKNYLA,
VLCDR2:
(SEQ ID NO: 14)
WASTRES
VLCDR3:
(SEQ ID NO: 37)
QQYYNYPYT

18. Binding molecule according to any one of items 3-17, wherein said VH region comprises or consists of an amino acid sequence selected from
(i) the group consisting of SEQ ID NO:44-57, 65 and 67, for example the group consisting of SEQ ID NO:44-57, for example the group consisting of SEQ ID NO:44 and 50; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

19. Binding molecule according to any one of items 3-18, wherein said VL region comprises or consists of an amino acid sequence selected from
(i) the group consisting of SEQ ID NO:58-64, 66 and 68, for example the group consisting of SEQ ID NO:58-64; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

20. Binding molecule according to any one of items 18-19, wherein said VH region is as defined in item 18 and said VL region is as defined in item 19.

21. Binding molecule according to item 20, in which said VH region comprises SEQ ID NO:44 and said VL region comprises a sequence selected from SEQ ID NO:58-64.

22. Binding molecule according to item 20, in which said VH region comprises a sequence selected from SEQ ID NO:44-57 and said VL region comprises SEQ ID NO:58.

23. Binding molecule according to any one of items 21-22, in which said VH region comprises SEQ ID NO:44 and said VL region comprises SEQ ID NO:58.

24. Binding molecule according to any one of items 21-22, in which said VH region comprises SEQ ID NO:50 and said VL region comprises SEQ ID NO:58.

25. Binding molecule according to item 3, in which said antigen-binding surface is composed of three complementarity-determining regions (CDRs) from said VH region and three CDRs from said VL region, and in which said CDRs comprise the following:

VHCDR1:
(SEQ ID NO: 38)
NYWLG,
VHCDR2:
(SEQ ID NO: 39)
DIFPGSDNTYYNEKFKG,
VHCDR3:
(SEQ ID NO: 40)
SGNFYAMDY
VLCDR1:
(SEQ ID NO: 41)
SASSSVNYMN,
VLCDR2:
(SEQ ID NO: 42)
DTSKLAS
VLCDR3:
(SEQ ID NO: 43)
FQGSGYPFT

26. Binding molecule according to item 25, wherein said VH region comprises or consists of an amino acid sequence selected from SEQ ID NO:69 and a sequence having at least 80%, at least 90%, at least 92%, at least 94%, at least 95%, at least 96%, at least 97%, at least 98% or at least 99% identity to SEQ ID NO:69, provided that the sequences of the CDR regions are 100% identical to those of SEQ ID NO:69.

27. Binding molecule according to any one of items 25-26, wherein said VL region comprises or consists of an amino acid sequence selected from SEQ ID NO:70 and a sequence having at least 80%, at least 90%, at least 92%, at least 94%, at least 95%, at least 96%, at least 97%, at least 98% or at least 99% identity to SEQ ID NO:70, provided that the sequences of the CDR regions are 100% identical to those of SEQ ID NO:70.

28. Binding molecule according to any one of items 26-27, wherein said VH region is as defined in item 26 and said VL region is as defined in item 27.

29. Binding molecule according to any one of items 3-28, in which said VH/VL pair forms part of an antibody construct.

30. Binding molecule according to item 29, in which said antibody construct has more than one binding specificity.

31. Binding molecule according to item 30, in which said antibody construct is bispecific.

32. Binding molecule according to item 31, wherein the VH/VL pair as defined in any one of items 3-28 is present in an antibody fragment selected from the group consisting of a Fab fragment, a single chain Fab (scFab) fragment, an Fv fragment and a single chain (scFv) fragment.

33. Binding molecule according to item 32, wherein said antibody fragment is an scFv.

34. Binding molecule according to any one of items 30-33, further comprising an antibody or antigen binding fragment thereof capable of selective binding to a target present in the brain of a mammal.

35. Binding molecule according to item 34, wherein said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-3 precursor protein, p75 neurotrophin receptor, neuregulin and caspase 6.

36. Binding molecule according to item 35, wherein said target is selected from the group consisting of amyloid-β peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), Tau, phosphorylated Tau or fragments thereof and apolipoprotein E4.

37. Binding molecule according to item 36, wherein said target is selected from the group consisting of amyloid-β peptide or derivatives or fragments thereof, alpha-synuclein or derivatives or fragments thereof and TAR DNA-binding protein 43 (TDP-43) or derivatives or fragments thereof.

38. Binding molecule according to any one of items 34-37, wherein said antibody or antigen binding fragment thereof capable of selective binding to a target present in the brain of a mammal is an anti-Aβ antibody, for example an antibody selected from the group consisting of lecanemab, gantenerumab, aducanumab, donanemab, PBD-C06 and KHK6640.

39. Binding molecule according to any one of items 34-37, wherein said antibody or antigen binding fragment thereof capable of selective binding to a target present in the brain of a mammal is an anti-alpha-synuclein antibody, for example an antibody selected from the group consisting of prasinezumab, UCB7853, Lu AF82422, TAK-341 and BAN0805.

40. Pharmaceutical composition, comprising a binding molecule according to any preceding item and a pharmaceutically acceptable carrier or excipient.

41. A binding molecule according to any one of items 1-39 or a composition according to item 40 for use in treatment, such as for use in therapeutic treatment or for use in prophylactic treatment.

42. A binding molecule according to any one of items 1-39 or a composition according to item 40 for use in diagnosis in vivo or prognosis in vivo.

43. A binding molecule or composition for use according to any one of items 41-42, wherein said therapy, prophylaxis, in vivo diagnosis or in vivo prognosis is with respect to a neurodegenerative disorder, for example a disorder selected from Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

44. A binding molecule or composition for use according to item 43, wherein said therapy, prophylaxis, in vivo diagnosis or in vivo prognosis is with respect to a disorder selected from Alzheimer's disease and other disorders associated with AB protein aggregation, Lewy body dementia (LBD), Down's syndrome (DS), amyotrophic lateral sclerosis (ALS), frontotemporal dementia, tauopathy, Parkinson's disease (PD), Parkinson's disease dementia (PDD) and the Lewy body variant of Alzheimer's disease.

45. A binding molecule or composition for use according to item 44, wherein said therapy, prophylaxis, in vivo diagnosis or in vivo prognosis is with respect to a disorder selected from Alzheimer's disease and other disorders associated with Aβ protein aggregation, Lewy body dementia (LBD), amyotrophic lateral sclerosis (ALS) and Parkinson's disease (PD).

46. A binding molecule or composition for use according to item 45, wherein said therapy, prophylaxis, in vivo diagnosis or in vivo prognosis is with respect to Alzheimer's disease.

47. A binding molecule or composition for use according to any one of items 41-42, wherein said therapy, prophylaxis, in vivo diagnosis or in vivo prognosis is with respect to a disorder selected from brain cancer, multiple sclerosis and lysosomal storage diseases.

48. A method of therapeutic or prophylactic treatment of a mammal having, or being at risk of developing, a disorder, said method comprising administering to said mammal a therapeutically effective amount of a binding molecule according to any one of items 1-39 or a composition according to item 40.

49. A method according to item 48, wherein said disorder is a neurodegenerative disorder, for example a neurodegenerative disorder as defined in any one of items 43-46.

50. A method according to item 48, wherein said disorder is as defined in item 47.

1. A transferrin receptor 1 (TfR1) binding molecule, capable of selective binding to an epitope located in the protease-like domain of TfR1 defined by amino acid residues 121-183 and 384-605 in SEQ ID NO:85.

2. Binding molecule according to claim 1, comprising an immunoglobulin heavy chain variable region (VH) and an immunoglobulin light chain variable region (VL), said VH and VL regions forming a VH/VL pair comprising an antigen-binding surface, in which said antigen-binding surface provides the binding molecule with the capacity to bind selectively to said epitope.

3. Binding molecule according to claim 2, in which said antigen-binding surface is composed of three complementarity-determining regions (CDRs) from said VH region and three CDRs from said VL region, and in which said CDRs comprise the following:
VHCDR1:
(SEQ ID NO: 1)
X1X2NMX3,
wherein
X1 is selected from D and A;
X2 is selected from Y and A; and
X3 is selected from D and A;
VHCDR2:
(SEQ ID NO: 2)
X4INPX5X6X7TTSX8X9X10KFKG,
wherein
X4 is selected from D and A;
X5 is selected from D, N and A;
X6 is selected from Y and A;
X7 is selected from D and A;
X8 is selected from Y and A;
X9 is selected from N and S; and
X10 is selected from E and Q;
VLCDR1:
(SEQ ID NO: 4)
KSSQSLLX11SX12NX13KNX14LA,
wherein
X11 is selected from Y and A;
X12 is selected from T and S;
X13 is selected from Q and R; and
X14 is selected from Y and A;
VLCDR2:
(SEQ ID NO: 5)
X15ASTRES
wherein
X15 is selected from W and A; and
VLCDR3:
(SEQ ID NO: 6)
QQX16X17X18X19PX20T
wherein
X16 is selected from Y and A;
X17 is selected from F and Y;
X18 is selected from I and N;
X19 is selected from Y and A; and
X20 is selected from R and Y;
optionally further comprising
VHCDR3:
(SEQ ID NO: 3)
GGX21SGSSX22X23HPMX24X25
wherein
X21 is selected from Y and A;
X22 is selected from Y and A;
X23 is selected from Y and A;
X24 is selected from D and A; and
X25 is selected from Y and A.

4. Binding molecule according to claim 3, in which the amino acid sequences of the six CDRs are the following:
VHCDR1:
(SEQ ID NO: 10)
DYNMD,
VHCDR2:
(SEQ ID NO: 11)
DINPDYDTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

5. Binding molecule according to claim 3, in which the amino acid sequences of the six CDRs are the following:
VHCDR1:
(SEQ ID NO: 10)
DYNMD,
VHCDR2:
(SEQ ID NO: 21)
DINPDADTTSYNEKFKG,
VHCDR3:
(SEQ ID NO: 12)
GGYSGSSYYHPMDY
VLCDR1:
(SEQ ID NO: 13)
KSSQSLLYSTNQKNYLA,
VLCDR2:
(SEQ ID NO: 14)
WASTRES
VLCDR3:
(SEQ ID NO: 15)
QQYFIYPRT

6. Binding molecule according to claim 2, wherein said VH region comprises or consists of an amino acid sequence selected from
(i) the group consisting of SEQ ID NO:44-57, 65 and 67; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

7. Binding molecule according to claim 2, wherein said VL region comprises or consists of an amino acid sequence selected from
(i) the group consisting of SEQ ID NO:58-64, 66 and 68; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

8. Binding molecule according to claim 2 wherein
said VH region comprises or consists of an amino acid sequence selected from
(i) the group consisting of SEQ ID NO:44-57, 65 and 67; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i); and wherein
said VL region comprises or consists of an amino acid sequence selected from
(i) the group consisting of SEQ ID NO:58-64, 66 and 68; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

9. Binding molecule according to claim 2, in which said VH/VL pair forms part of an antibody construct.

10. Binding molecule according to claim 9, wherein the VH/VL pair is present in an antibody fragment selected from the group consisting of a Fab fragment, a single chain Fab (scFab) fragment, an Fv fragment and a single chain (scFv) fragment.

11. Binding molecule according to claim 9, further comprising an antibody or antigen binding fragment thereof capable of selective binding to a target present in the brain of a mammal.

12. Pharmaceutical composition, comprising a binding molecule according to claim 1 and a pharmaceutically acceptable carrier or excipient.

13. A method of treating a neurodegenerative disorder comprising administering to a subject a therapeutically effective amount of the binding molecule of claim 1.

14. The method according to claim 13, wherein the neurodegenerative disorder is selected from the group consisting of Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

15. A method of treating a neurodegenerative disorder comprising administering to a subject a therapeutically effective amount of the composition of claim 12.

16. The method of claim 15, wherein the neurodegenerative disorder is selected from the group consisting of Alzheimer's disease and other disorders associated with Aβ protein aggregation, traumatic brain injury (TBI), Lewy body dementia (LBD), Down's syndrome (DS), amyotrophic lateral sclerosis (ALS), frontotemporal dementia, tauopathy, systemic amyloidosis, atherosclerosis, Parkinson's disease (PD), Parkinson's disease dementia (PDD), the Lewy body variant of Alzheimer's disease, multiple system atrophy, psychosis, schizophrenia, Creutzfeldt-Jakob disease, Huntington's disease, and familial amyloid neuropathy.

17. Binding molecule according to claim 6, wherein said VH region comprises or consists of an amino acid sequence selected from
i) the group consisting of SEQ ID NO:44-57; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

18. Binding molecule according to claim 17, wherein said VH region comprises or consists of an amino acid sequence selected from
i) the group consisting of SEQ ID NO:44 and SEQ ID NO: 50; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

19. Binding molecule according to claim 7 wherein said VL region comprises or consists of an amino acid sequence selected from
i) the group consisting of SEQ ID NO:58-64; and
(ii) a sequence having at least 80%, at least 90%, at least 92%, at least 94%, at least 95%, at least 96%, at least 97%, at least 98% or at least 99% identity to a sequence defined in (i), provided that the sequences of the CDR regions are 100% identical to those of a sequence defined in (i).

20. Binding molecule according to claim 9, wherein said antibody construct is a bispecific antibody construct.

21. Binding molecule according to claim 10, wherein the VH/VL pair is present in an scFv.

22. Binding molecule according to claim 11, wherein said target is selected from the group consisting of amyloid-R peptide or derivatives or fragments thereof, alpha-synuclein or derivatives or fragments thereof, TAR DNA-binding protein 43 (TDP-43) or derivatives or fragments thereof, triggering receptor expressed on myeloid cells 2 (TREM2), beta-secretase 1 (BACE1), superoxide dismutase (SOD), huntingtin, transthyretin, P-secretase 1, epidermal growth factor, epidermal growth factor receptor 2, Tau, phosphorylated Tau or fragments thereof, apolipoprotein E4, CD20, prion protein, leucine rich repeat kinase 2, parkin, presenilin 2, gamma secretase, death receptor 6, amyloid-β precursor protein, p75 neurotrophin receptor, neuregulin and caspase 6.

23. Binding molecule according to claim 1, wherein said epitope comprises or consists of the amino acid residues 150, 151, 154, 158, 159, 161, 163 and 385 in SEQ ID NO:85.
